(19) United States
(12) Patent Application Publication
(10) Pub. No.: US 2023/0250145 A1
(43) Pub. Date: Aug. 10, 2023

(54) COMPOSITIONS AND METHODS FOR THE TREATMENT OF CANCER

(71) Applicant: MOMA Therapeutics, Inc., Brighton, MA (US)

(72) Inventors: Andrew GARNER, Brighton, MA (US); Agustin CHICAS, Brighton, MA (US); David ORLANDO, Brighton, MA (US)

(21) Appl. No.: 18/010,437

(22) PCT Filed: Jun. 23, 2021
(86) PCT No.: PCT/US2021/038767

§ 371 Date: Dec. 14, 2022

Related U.S. Application Data
(60) Provisional application No. 63/043,430, filed on Jun. 24, 2020.

(51) Int. Cl. C07K 14/47; C12N 15/113; A61P 35/00

(52) U.S. Cl. CPC C07K 14/4702; C12N 15/113; A61P 35/00

(57) ABSTRACT

The disclosure provides compositions and methods for the selective targeting of components of a first or a second DNA repair pathway in cancer cells having an impaired, defective or deregulated first DNA repair pathway. In some embodiments of the disclosure, the disclosure provides a composition comprising a DNA ligase 1 (LIG1) blocking agent, wherein in a target cell comprising an impaired, defective or deregulated homologous recombination (HR) repair pathway, the blocking agent reduces or inhibits a function of LIG1. In some embodiments of the disclosure, the disclosure provides composition comprising a Fanconi Anemia Group M protein (FANCM) blocking agent, wherein in a target cell comprising an impaired, defective or deregulated homologous recombination (HR) repair pathway, the blocking agent reduces or inhibits a function of FANCM. The disclosure further provides methods for using compositions of the disclosure for the treatment of cancer.

### CROSS-REFERENCE TO RELATED APPLICATIONS

This application claims priority to U.S. Provisional Patent Application Ser. No. 63/043,430, filed Jun. 24, 2020, which is incorporated herein by reference in its entirety.

### SEQUENCE LISTING

This application is being filed electronically via EFS-Web and includes an electronically submitted sequence listing in .txt format. The .txt file contains a sequence listing entitled MOMA_001_01WO_ST25.txt created on Jun. 22, 2021 and having a size of 430 kilobytes. The sequence listing contained in this .txt file is part of the specification and is incorporated herein by reference in its entirety.

### FIELD OF THE DISCLOSURE

The disclosure is directed to compositions and methods for the targeting of DNA repair pathway(s) to treat cancer.

### BACKGROUND

There is a long-felt and unmet need in the art for an effective and safe cancer therapeutic. The disclosure provides compositions and methods for the selective targeting of components of a first or a second DNA repair pathway in cancer cells in which a first DNA repair pathway is impaired, defective or deregulated.

### SUMMARY

The disclosure provides a composition comprising a DNA ligase 1 (LIG1) blocking agent, wherein in a target cell comprising an impaired homologous recombination (HR) repair pathway, the blocking agent reduces or inhibits a function of LIG1.

The disclosure provides a composition comprising a DNA ligase 1 (LIG1) blocking agent, wherein in a target cell comprising a defective homologous recombination (HR) repair pathway, the blocking agent reduces or inhibits a function of LIG1.

The disclosure provides a composition comprising a DNA ligase 1 (LIG1) blocking agent, wherein in a target cell comprising deregulated homologous recombination (HR) repair pathway, the blocking agent reduces or inhibits a function of LIG1.

In some embodiments of the compositions of the disclosure, a LIG1 blocking agent comprises a LIG1 inhibitor.

The disclosure provides a composition comprising a Fanconi Anemia Group M protein (FANCM) blocking agent, wherein in a target cell comprising an impaired homologous recombination (HR) repair pathway, the blocking agent reduces or inhibits a function of FANCM.

The disclosure provides a composition comprising a Fanconi Anemia Group M protein (FANCM) blocking agent, wherein in a target cell comprising a defective homologous recombination (HR) repair pathway, the blocking agent reduces or inhibits a function of FANCM.

The disclosure provides a composition comprising a Fanconi Anemia Group M protein (FANCM) blocking agent, wherein in a target cell comprising a deregulated homologous recombination (HR) repair pathway, the blocking agent reduces or inhibits a function of FANCM.

In some embodiments of the compositions of the disclosure, a FANCM blocking agent comprises a FANCM inhibitor.

Impaired homologous recombination (HR) repair pathways of the disclosure include those pathways wherein one or more components (e.g. signaling proteins/enzymes) of the pathway are impaired. In some embodiments, an HR repair pathway or a component thereof is a variant component that demonstrates an altered function or an altered activity compared to a non-variant or wild type component and which, in the context of the HR repair pathway, impairs a function or an activity of the HR repair pathway. In some embodiments, when a variant component demonstrates an altered function or an altered activity compared to a non-variant or wild type component the altered function or altered activity may be a decrease or loss of the function or the activity. In some embodiments, when a variant component demonstrates an altered function or an altered activity compared to a non-variant or wild type component the altered function or altered activity may be an increase or a gain of the function or of an activity. In some embodiments, an impairment of a pathway (including an HR pathway) or a component thereof comprises a complete loss of function, rendering the pathway or component defective. In some embodiments, an impairment of a pathway (including an HR pathway) or a component thereof comprises a deregulation of the pathway or a component thereof. In some embodiments, an impaired, defective or deregulated pathway or component thereof induces a disease or disorder of the disclosure. In some embodiments, an impaired, defective or deregulated HR pathway or component thereof induces a disease or disorder of the disclosure. In some embodiments, an impaired, defective or deregulated pathway or component thereof induces a malignant transformation of cell and the onset of a cancer. In some embodiments, an impaired, defective or deregulated HR pathway or component thereof induces a malignant transformation of cell and the onset of a cancer. In some embodiments, an HR pathway of a cell is impaired, defective or deregulated. In some embodiments, a component of an HR pathway of a cell is impaired, defective or deregulated. In some embodiments, the component comprises a variant protein of the disclosure.

In some embodiments of the compositions of the disclosure, the target cell does not comprise a variant Breast Cancer (BRCA) BRCA protein or a sequence encoding a variant BRCA protein, and the variant BRCA protein induces a loss or reduction in a function of the HR pathway. In some embodiments, the target cell is a proliferating cell. In some embodiments, the target cell is a tumor cell. In some embodiments, the target cell is a malignant cell. In some embodiments, the target cell is a metastatic cell. In some embodiments, the target cell is produced or derived from a Ewing Sarcoma or sarcoma cell.

In some embodiments of the compositions of the disclosure, the target cell comprises a variant BRCA protein or a sequence encoding a variant BRCA protein and the variant BRCA protein induces a loss or reduction in a function of the HR pathway. In some embodiments, the variant BRCA protein comprises a variant BRCA1 protein or wherein the sequence encoding the variant BRCA protein comprises a sequence encoding a variant BRCA1 protein. In some embodiments, the variant BRCA protein comprises a variant BRCA2 protein or wherein the sequence encoding the variant BRCA protein comprises a sequence encoding a variant BRCA2 protein. In some embodiments, the target cell is a proliferating cell. In some embodiments, the target cell is a tumor cell. In some embodiments, the target cell is a malignant cell. In some embodiments, the target cell is a metastatic cell. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant DNA repair protein RAD51 homolog 1 (RAD51) or a variant homolog of RAD51 and the variant RAD51 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant DNA repair protein RAD51 homolog 3 (RAD51C) or a variant homolog of RAD51C and the variant RAD51C induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant DNA repair protein RAD51 homolog 4 (RAD51D) or a variant homolog of RAD51D and the variant RAD51D induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant X-ray repair cross-complementing 2 (XRCC2) or a variant homolog of XRCC2 and the variant XRCC2 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant DNA repair endonuclease XPF or a variant homolog of XPF and the variant XPF induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Meiotic recombination 11 homolog 1 (MRE11A) or a variant homolog of MRE11A and the variant MRE11A induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Ataxia telangiectasia mutated (ATM) or a variant homolog of ATM and the variant ATM induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant BRCA1-associated RING domain protein 1 (BARD1) or a variant homolog of BARD1 and the variant BARD1 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant BRCA1-interacting protein C-terminal helicase 1 (BRIP1) or a variant homolog of BRIP1 and the variant BRIP1 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Cell cycle checkpoint kinase (CHEK1) or a variant homolog of CHEK1 and the variant CHEK1 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant CHEK1 checkpoint homolog (CHEK2) or a variant homolog of CHEK2 and the variant CHEK2 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Nibrin (NBN) or a variant homolog of NBN and wherein the variant NBN induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Partner and localizer of BRCA2 (PALB2) or a variant homolog of PALB2 and wherein the variant PALB2 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the target cell comprises a nucleic acid or an amino acid encoding a variant Structure-specific endonuclease subunit SLX4 (SLX4) or a variant homolog of SLX4 and the variant SLX4 induces a loss or reduction in a function of the HR pathway. In some embodiments of the compositions of the disclosure, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment. In some embodiments, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, including those embodiments in which the target cell comprises a variant protein or a sequence encoding a variant protein and in which the variant protein transduces an intracellular signal in an HR pathway, the variant protein or the sequence encoding the variant protein comprises one or more of a mutation, a deletion, a promotor methylation, a silencing event and a splicing event. In some embodiments, the mutation comprises one or more of a substitution, an insertion, a deletion, an inversion, and a translocation of a nucleic acid sequence or an amino acid sequence encoding the variant protein. In some embodiments, the mutation introduces a stop codon into a nucleic acid sequence encoding the variant protein, thereby generating one or more of a truncated protein, an inactivated protein and a protein fragment.

In some embodiments of the compositions of the disclosure, including those embodiments in which the target cell comprises a variant protein or a sequence encoding a variant protein and in which the variant protein transduces an intracellular signal in an HR pathway, the variant protein or the sequence encoding the variant protein comprises a promoter sequence and wherein the promoter controls expression of the variant protein or the sequence encoding the variant protein. In some embodiments, the silencing event comprises a silencing of the promoter sequence, function, or activity. In some embodiments, the promoter sequence controlling expression of the variant protein or the sequence encoding the variant protein comprises a mutation. In some embodiments, the mutation comprises one or more of a substitution, an insertion, a deletion, an inversion, and a translocation of the promoter sequence.

In some embodiments of the compositions of the disclosure, the impairment, defect or deregulation of the HR pathway increases a dependence of the target cell upon a function of LIG1. In some embodiments, the function of LIG1 comprises one or more of ATP-binding, magnesium-binding, metal-binding, nucleotide-binding and nucleotide ligation. In some embodiments, LIG1 functions as a ligase. In some embodiments, an activity of LIG1 comprises an increase or a decrease in a function of LIG1. In some embodiments, the variant protein increases a function of LIG1. In some embodiments, the variant protein decreases a function of LIG1.

In some embodiments of the compositions of the disclosure, the impairment, defect or deregulation of the HR pathway increases a dependence of the target cell upon a function of FANCM. In some embodiments, the function of FANCM comprises one or more of ATP-binding, nucleotide-binding, DNA-binding, DNA remodeling, DNA strand separation, DNA-RNA strand separation and catalyzing the break of a chemical bond using water. In some embodiments, FANCM functions as a helicase. In some embodiments, FANCM functions as a hydrolase. In some embodiments, FANCM functions as a translocase. In some embodiments, an activity of FANCM comprises an increase or a decrease in a function of FANCM. In some embodiments, the variant protein increases a function of FANCM. In some embodiments, the variant protein decreases a function of FANCM.

In some embodiments of the compositions of the disclosure, the function of the HR pathway comprises one or more of i) recognizing nucleotide or DNA damage; ii) recruiting a protein to a site of nucleotide or DNA damage; iii) configuring or remodeling a sequence comprising a site of nucleotide or DNA damage; iv) configuring or remodeling a sequence complementary to a site of nucleotide or DNA damage; v) inducing a break in a sequence within a site of nucleotide or DNA damage; vi) inducing a break in a sequence comprising the site of nucleotide or DNA damage; vii) inducing a break in a sequence complementary to a site of nucleotide or DNA damage; viii) removing a sequence within a site of nucleotide or DNA damage; ix) removing a sequence comprising a site of nucleotide or DNA damage; x) synthesizing a new sequence within a site of nucleotide or DNA damage; xi) synthesizing a new sequence comprising a site of nucleotide or DNA damage; xii) resecting a portion of a synthesized sequence within a site of nucleotide or DNA damage; xiii) resecting a portion of a synthesized sequence comprising the site of nucleotide or DNA damage; xiv) stabilizing a site of DNA synthesis or replication within a site of nucleotide or DNA damage; xv) stabilizing a site of DNA synthesis or replication comprising a site of nucleotide or DNA damage; xvi) stabilizing a site of DNA synthesis or replication comprising a target site; xvii) stabilizing a site of DNA synthesis or replication comprising a stalled replication fork; xviii) inducing or facilitating invasion of a synthesized sequence within the site of nucleotide or DNA damage; xix) inducing or facilitating invasion of a synthesized sequence comprising the site of nucleotide or DNA damage; xx) inducing or facilitating insertion of a synthesized sequence within the site of nucleotide or DNA damage by recombination; and xxi) inducing or facilitating insertion of a synthesized sequence comprising the site of nucleotide or DNA damage by recombination.

In some embodiments of the compositions of the disclosure, an activity of the HR pathway comprises an increase or a decrease in a function of a component of the HR pathway. In some embodiments, the variant protein increases a function of a component of the HR pathway. In some embodiments, the variant protein decreases a function of a component of the HR pathway.

In some embodiments of the compositions of the disclosure, the composition further comprises a pharmaceutically acceptable carrier.

In some embodiments of the compositions of the disclosure, the blocking agent comprises an effector moiety that binds to a LIG-1 protein. In some embodiments, the effector moiety comprises one or more of an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

In some embodiments of the compositions of the disclosure, the blocking agent comprises an effector moiety that binds to a FANCM protein or a nucleic acid sequence encoding the FANCM protein. In some embodiments, the effector moiety comprises one or more of an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

In some embodiments of the compositions of the disclosure, including those in which the blocking agent comprises an effector moiety, the blocking agent further comprises a targeting moiety operably linked to the effector moiety. In some embodiments, the targeting moiety is reversibly linked to the effector moiety. In some embodiments, the targeting moiety specifically binds a component of the target cell. In some embodiments, the target cell is a proliferating cell. In some embodiments, the target cell is a tumor cell. In some embodiments, the target cell is a malignant cell. In some embodiments, the target cell is a metastatic cell. In some embodiments, the target cell is produced or derived from a non-hematological tissue. In some embodiments, the target cell is produced or derived from an epithelial tissue. In some embodiments, the target cell is produced or derived from an organ or a structure comprising an epithelial tissue. In some embodiments, the target cell is produced or derived from a skin area, a skin layer, a lung, a lymph node, a breast, an ovary, a prostate, a mouth, a nose, a nasal passage, an esophagus, an intestine, a small intestine, a large intestine, a stomach, a kidney, a liver, a spleen, a heart, an artery, a vein, a bladder and a colon. In some embodiments, the target cell is produced or derived from a bone or a connective tissue.

In some embodiments of the compositions of the disclosure, including those in which the blocking agent comprises one or more of an effector moiety and a targeting moiety, the blocking agent further comprises a regulation moiety. In some embodiments, the regulation moiety is operably linked to one or more of the effector moiety and the targeting moiety. In some embodiments, the regulation moiety is reversibly linked to one or more of the effector moiety and the targeting moiety. In some embodiments, the regulation moiety selectively binds a component not present in a target cell. In some embodiments, the component not present in a target cell is present in a healthy cell. In some embodiments, the component decreases or inhibits an activity of the effector moiety. In some embodiments, the regulation moiety comprises a microRNA (miRNA) binding site and selectively binds a miRNA.

The disclosure provides a method of inducing cell death in a proliferating cell, comprising contacting the proliferating cell and the composition of the disclosure. In some embodiments, the cell is in vitro or ex vivo. In some embodiments, the cell is in vivo. In some embodiments, the proliferating cell is a target cell of the disclosure.

The disclosure provides a method of inducing cell cycle arrest in a proliferating cell, comprising contacting the proliferating cell and a composition of the disclosure. In some embodiments, the cell is in vitro or ex vivo. In some embodiments, the cell is in vivo. In some embodiments, the proliferating cell is a target cell of the disclosure.

The disclosure provides a method of arresting growth of a proliferating cell, comprising contacting the proliferating cell and a composition of the disclosure. In some embodiments, the cell is in vitro or ex vivo. In some embodiments, the cell is in vivo. In some embodiments, the proliferating cell is a target cell of the disclosure. In some embodiments, arrest growth comprises delaying the onset, reducing a probability or inhibiting the entry of the cell entering the cell cycle following contact with a composition of the disclosure. In some embodiments, prior to contacting the composition, the cell had undergone at least one proliferation, however, subsequent to contacting the composition, the proliferating or target cell does not enter the cell cycle again. In some embodiments, the proliferating or target cell never enters the cell cycle for a second proliferation. In some embodiments, the proliferating or target cell delays entry into the cell cycle for a period of time. In some embodiments, the proliferating or target cell delays entry into the cell cycle for at least 30 minutes, at least 1 hour, at least 12 hours, at least one day (24 hours), at least 2 days, at least 1 week, at least 1 month or at least any period of time in between. In some embodiments, contact with a composition of the disclosure decreases or reduces a probability that the proliferating or target cell will ever enter the cell cycle or that the proliferating or target cell will enter the cell cycle within a period of time. In some embodiments, contact with a composition of the disclosure decreases or reduces a probability that the proliferating or target cell will enter the cell cycle for at least 30 minutes, at least 1 hour, at least 12 hours, at least one day (24 hours), at least 2 days, at least 1 week, at least 1 month or at least any period of time in between. In some embodiments, following contact with a composition of the disclosure, the target cell undergoes necrosis, apoptosis or another form of cell death. In some embodiments, following contact with a composition of the disclosure, the target cell enters senescence.

The disclosure provides a method of inhibiting proliferation of a proliferating cell, comprising contacting the proliferating cell and a composition of the disclosure. In some embodiments, the cell is in vitro or ex vivo. In some embodiments, the cell is in vivo. In some embodiments, the proliferating cell is a target cell of the disclosure. In some embodiments, prior to contacting the composition, the cell had undergone at least one proliferation, however, subsequent to contacting the composition, the proliferating or target cell does not proliferate again. In some embodiments, following contact with a composition of the disclosure, the target cell undergoes necrosis, apoptosis or another form of cell death. In some embodiments, following contact with a composition of the disclosure, the target cell enters senescence.

The disclosure provides a method of inducing senescence of a proliferating cell, comprising contacting the proliferating cell and a composition of the disclosure. In some embodiments, the cell is in vitro or ex vivo. In some embodiments, the cell is in vivo. In some embodiments, the proliferating cell is a target cell of the disclosure. In some embodiments, prior to contacting the composition, the cell had undergone at least one proliferation, however, subsequent to contacting the composition, the proliferating or target cell enters senescence. In some embodiments, senescence comprises an irreversible cell cycle arrest. In some embodiments, senescence further comprises one or more of telomere shortening, oxidative stress, genotoxic stress, increase in the expression or activity of one or more inflammatory agents (e.g. mitogens or inflammatory cytokines), an activation of the p53 tumor suppressor, and an activation of the cyclin-dependent kinase inhibitor p16. In some embodiments, oxidative stress comprises an imbalance between a production of free radicals and a neutralization of the free radicals by antioxidants. In some embodiments, genotoxic stress comprises cellular exposure to DNA-damaging agents and, optionally, increased energy expenditures by the cell to repair DNA damage.

The disclosure provides a method of treating cancer, comprising administrating to a subject an effective amount of the composition of the disclosure. In some embodiments, the method further comprises administering a second therapy. In some embodiments, the second therapy comprises radiation and/or a chemotherapy. In some embodiments, the chemotherapy comprises a Poly (ADP-ribose) polymerase (PARP) inhibitor or a platinum-based therapy. In some embodiments, the cancer is resistant to treatment with a PARP inhibitor as a monotherapy. In some embodiments, prior to administration of the composition, the subject has been identified as resistant to treatment with a PARP inhibitor as a monotherapy. In some embodiments, prior to administration of the composition, the subject has been treated with a PARP inhibitor as a monotherapy.

In some embodiments of the methods of the disclosure, the administration is systemic. In some embodiments, the composition is administered by one or more of an oral route, an inhaled route, an intravenous route, an intraperitoneal route, and a subcutaneous route.

In some embodiments of the methods of the disclosure, the administration is local. In some embodiments, the composition is administered by one or more of an intraocular route, an intraspinal route, an intracerebellar route, an intrathecal route, an intramuscular route and an intraosseous route.

In some embodiments of the methods of the disclosure, the composition is administered once per day, twice per day or three times per day. In some embodiments, the composition is administered once per week, twice per week or three times per week. In some embodiments, the composition is administered once per month, twice per month or three times per month.

In some embodiments of the methods of the disclosure, treating comprises a reduction in a severity of a sign or symptom of the cancer. In some embodiments, treating comprises a reduction in a volume of a tumor. In some embodiments, treating comprises a reduction in a number of tumor cells per volume of blood or mass of tissue. In some embodiments, treating comprises a remission. In some embodiments, treating comprises an increased duration of progression free survival.

In some embodiments of the methods of the disclosure, including the methods of treating a cancer, the cancer comprises cancer cells comprising an impaired, defective or deregulated DNA repair pathway. In some embodiments, the cancer cells comprise an impaired, defective or deregulated homologous repair (homologous recombination (HR) repair pathway. In some embodiments, the cancer cells comprise a variant BRCA protein or a sequence encoding a variant BRCA protein, wherein the variant BRCA protein induces a loss or reduction in a function of the HR pathway. In some embodiments, the variant BRCA protein comprises a variant BRCA1 protein or a variant BRCA2 protein, or the sequence encoding the variant BRCA protein comprises a sequence encoding a variant BRCA1 protein or a variant BRCA2 protein. In some embodiments, the cancer cells are ALT+ cells. In some embodiments, the cancer cells are not an ALT+ cells. In some embodiments, the cancer cells are resistant to a PARP inhibitor. In some embodiments, the cancer is an ovarian cancer or a breast cancer, optionally a BRCA1^{-/-} cancer or a cancer comprising tumor cells comprising a variant BRCA1 protein, or a sequence encoding the variant BRCA protein. In some embodiments, the method comprises administering to a subject having a BRCA1−/− ovarian cancer or BRCA1−/− breast cancer a Fanconi Anemia Group M protein (FANCM) blocking agent. In some embodiments, the method results in DNA damage and/or cell cycle arrest of BRCA1−/− ovarian cancer cells or BRCA1−/− breast cancer cells. In some embodiments, the method decreases survival or growth of BRCA1−/− ovarian cancer cells or BRCA1−/− breast cancer cells.

### BRIEF DESCRIPTION OF THE DRAWINGS

FIG. 1 is a schematic diagram depicting a mechanism by which a FANCM blocking agent induces uncontrolled ALT and DNA damage in ALT+ cells.

FIG. 2 is a schematic diagram depicting the ligation mechanism of LIG1 that is common to all LIG1 isoforms.

FIGS. 3A-3B show that CRISPR/Cas9-mediated knockout of FANCM in BRCA1−/− cancer cell lines leads to an increase in DNA damage and G2 arrest. FIG. 3A is an overlay of flow cytometry contour plots of DNA damage marker γH2AX in TOV21G (BRCA1-proficient ovarian cancer), MDAMB436 (BRCA1−/− breast cancer) and UWB1289 (BRCA1−/− ovarian cancer) on day 7 post nucleofection with CRISPR RNP with sgRNA targeting FANCM. Cells, positive for γH2AX are selected with the gate and their percentage is indicated in the table above the plot. FIG. 3B provides cell cycle profiles of TOV21G and UWB1289 on day 7, following CRISPR/Cas9-mediated knockout of FANCM. The percentage of G2 cells in each sample is shown in the table.; NT=non-targeting gRNA control.

FIGS. 4A-4B show that CRISPR/Cas9-mediated knockout of FANCM in BRCA1−/− cancer cell lines leads to a reduction of growth in 2D clonogenic assay. FIG. 4A shows the results of a 2D clonogenic growth assay of UWB1.289 (BRCA1−/− ovarian cancer), COV362 (BRCA1−/− ovarian cancer) MDAMB436 (BRCA1−/− breast cancer), and TOV21G (BRCA1 wild-type ovarian cancer) on between 14-21 days post nucleofection with CRISPR RNP, targeting FANCM. FIG. 4B is a Western blot demonstrating loss of FANCM protein with FANCM sgRNA #3 and #4.

FIGS. 5A-5B show that shRNA mediated knockdown of FANCM in BRCA1−/− cancer cell lines leads reduction of growth in 2D clonogenic assay. FIG. 5A shows the results of a clonogenic growth assay of UWB1.289 (BRCA1−/− ovarian cancer) and MDAMB436 (BRCA1−/− breast cancer), and TOV21G (BRCA1 wild-type ovarian cancer) between 14-21 days after doxycycline induced shRNA expression. FIG. 5B shows qPCR results demonstrating loss of FANCM mRNA transcript in UWB1.289 with FANCM shRNA #1307 and #1858 follow 3 days of doxycycline treatment.

FIGS. 6A-6C show that CRISPR/Cas9-mediated knockout of FANCM in BRCA1−/− cancer cell line UWB1.289 leads to a reduction in colony formation and cell viability and is partially rescued by wild-type BRCA1 add back. FIGS. 6A and 6B show the results of short- and long-term viability assay demonstrates rescue of FANCM dependency when wild-type BRCA1 is present in UWB1.289 cells. FIG. 6C shows nuclear enriched FANCM is undetectable in both UWB1.289 null and BRCA1 add-back lines via western blot. In FIG. 6A, for each treatment, the bars from left to right correspond to UWB1.289 and UWB1.289+BRCA1, respectively.

### DETAILED DESCRIPTION

Disclosed are compositions and methods for the selective targeting of components of a first or a second DNA repair pathway in cancer cells in which a first DNA repair pathway is impaired, defective or deregulated. In certain embodiments, selective targeting refers to the use of blocking agents that specifically or selectively target a particular DNA repair pathway or component thereof, e.g., to inhibit the DNA repair pathway. In certain embodiments, the selectively targeted blocking agent does not substantially or significantly target, e.g., inhibit, other DNA repair pathways.

### DNA Repair

DNA repair may be accomplished by a number of pathways including, but not limited to, base excision repair (BER), nucleotide excision repair (NER), mismatch repair (MMR), homologous recombination (HR), and non-homologous end joining (NHEJ). These diverse pathways are active throughout the stages of the cell cycle in normal or healthy cells. In healthy cells (a cell in which all DNA repair pathways are fully active), at checkpoints within the cell cycle, DNA damage or replication mistakes are assessed and corrected prior to the next phase or to completion of the cycle. When DNA damage is present and DNA repair pathways are impaired, defective or deregulated, a healthy cell may initiate a programmed cell death or a cell cycle arrest.

Because there is redundancy in the DNA repair system, the impairment of one pathway among several pathways may not lead to apoptosis, but, rather, may lead to increased mutagenesis. Alternatively, or in addition, a loss of function of one component within a pathway may not be sufficient to completely inhibit that DNA repair pathway, leading instead to a functional impairment. In some circumstances, the impairment of a DNA repair pathway may lead to the generation of a malignant cell. Malignant cells of the disclosure may have one or more impaired, defective or deregulated DNA repair pathways. Alternatively, or in addition, malignant cells of the disclosure may have one or more components of the same DNA repair pathway in which a function is lost or reduced or the activity of that function is changed compared to a level of function or activity of that component in a non-malignant cell.

In various embodiments, an impaired, defective, or deregulated DNA repair pathway may be the result of one or more deletions or mutations in a gene encoding a protein in a DNA repair pathway. In certain embodiments, at least a portion of the coding region of a gene encoding a protein in a DNA repair pathway is partially or entirely deleted, resulting in expression of no protein, a truncated protein, or a mutant protein. In certain embodiments, a gene encoding a protein in a DNA repair pathway comprises one or more nucleic acid modification, such as, e.g., an insertion, deletion, or substitution, which may result in the encoded protein comprising one or more amino acid modification, such as a deletion, insertion, or substitution. The encoded protein may result from a frameshift mutation that results in at least a portion of the encoded protein being different than the wild type protein. In certain embodiments, an impaired, defective, or deregulated DNA repair pathway may be the result of a mutation in a regulatory region of a gene encoding a protein in a DNA repair pathway. For example, a promoter region may comprise one or more nucleic acid modification, such as a deletion, insertion or nucleotide substitution that results in reduced or no expression of the protein in the DNA repair pathway. In certain embodiments, the impaired, defective, or deregulated DNA repair pathway results in reduced or no activity of one or more protein in the DNA repair pathay, e.g., less than 90%, less than 80%, less than 70%, less than 60%, less than 50%, less than 40%, less than 30%, less than 20%, or less than 10% activity as compared to the wild type protein. In certain embodiments, the impaired, defective, or deregulated DNA repair pathway results in reduced or no expression of one or more protein in the DNA repair pathay, e.g., less than 90%, less than 80%, less than 70%, less than 60%, less than 50%, less than 40%, less than 30%, les than 20%, or less than 10% protein expression as compared to the amount expressed in a corresponding wild type cell.

In some embodiments, a malignant cell is targeted by a composition of the disclosure (i.e., a target cell) and the malignant or target cell may have an impaired, defective or deregulated homologous recombination (HR) pathway. In some embodiments, a malignant cell is targeted by a composition of the disclosure (i.e., a target cell) and the malignant or target cell may have an impaired, defective or deregulated base excision repair (BER) pathway. In some embodiments, a malignant cell is targeted by a composition of the disclosure (i.e., a target cell) and the malignant or target cell may have an impaired, defective or deregulated nucleotide excision repair (NER) pathway. In some embodiments, a malignant cell is targeted by a composition of the disclosure (i.e., a target cell) and the malignant or target cell may have an impaired, defective or deregulated mismatch repair (MMR) pathway. In some embodiments, a malignant cell is targeted by a composition of the disclosure (i.e., a target cell) and the malignant or target cell may have an impaired, defective or deregulated non-homologous end joining (NHEJ) pathway.

In some embodiments, compositions and methods of the disclosure target a second DNA repair pathway in a cell having an impaired, defective or deregulated first DNA repair pathway. The loss of two DNA repair pathways induces a synergistic effect on selectively and specifically targeting malignant cells to induce cell death or a cell cycle arrest in the malignant cell.

In certain embodiments, compositions and methods of the disclosure may target one of or any two components of the homologous recombination pathway. In some embodiments, compositions and methods of the disclosure may target one of or any two components of the homologous recombination pathway, including, but not limited to, BRCA1, BRCA2, LIG1, FANCM, RAD51, RAD51C, RAD51D, XRCC2, XPF, MRE11A, ATM, BARD1, BRIP1, CHEK1, CHEK2, NBN, PALB2 and SLX4. In some embodiments, compositions and methods of the disclosure may target LIG1 in a cell comprising a variant protein that impairs a function or an activity of the function of the homologous recombination (HR) pathway wherein the variant protein is a variant of one or more of: BRCA1, BRCA2, RAD51, RAD51C, RAD51D, XRCC2, XPF, MRE11A, ATM, BARD1, BRIP1, CHEK1, CHEK2, NBN, PALB2 and SLX4. In some embodiments, compositions and methods of the disclosure may target FANCM in a cell comprising a variant protein that impairs a function or an activity of the function of the homologous recombination (HR) pathway wherein the variant protein is a variant of one or more of: BRCA1, BRCA2, RAD51, RAD51C, RAD51D, XRCC2, XPF, MRE11A, ATM, BARD1, BRIP1, CHEK1, CHEK2, NBN, PALB2 and SLX4. In some embodiments, compositions and methods of the disclosure may target FANCM in a cell comprising a variant protein that impairs a function or an activity of the function of the homologous recombination (HR) repair pathway wherein the variant protein is a variant of BRCA1 or BRCA2. In some embodiments, compositions and methods of the disclosure may target LIG1 and FANCM in a cell comprising a variant protein that impairs a function or an activity of the function of the homologous recombination (HR) pathway wherein the variant protein is a variant of one or more of: BRCA1, BRCA2, RAD51, RAD51C, RAD51D, XRCC2, XPF, MRE11A, ATM, BARD1, BRIP1, CHEK1, CHEK2, NBN, PALB2 and SLX4.

### Homologous Recombination

Homologous recombination (HR) is a process interrelated with one or more DNA repair pathways that target sites including, but not limited to, DNA double-stranded breaks (DSBs) and interstrand crosslinks (ICLs). Additionally, the HR pathway stabilizes replication forks during DNA synthesis.

In some embodiments of the disclosure, LIG1 blocking agents of the disclosure block the ability of LIG1 to join unligated DNA fragments, which results in an elevation of single strand breaks that causes replication fork collapse, DNA damage and reduced cell growth in cells that are deficient in homologous recombination. In some embodiments, the LIG1 blocking agent is a selective LIG1 blocking agent.

In some embodiments of the disclosure, FANCM blocking agents of the disclosure block the ability of FANCM to resolve stalled replication forks, which in the context of homologous recombination deficiency results in elevated DNA damage and reduced cell growth. In some embodiments, the FANCM blocking agent is a selective FANCM blocking agent.

Target cells of the disclosure may have an impaired, defective or deregulated HR pathway (which may also be referred to as HR repair pathway).

Target cells of the disclosure may express one or more variant proteins including, but not limited to, one or more BRCA1, BRCA2, RAD51, RAD51C, RAD51D, XRCC2, XPF, MRE11A, ATM, BARD1, BRIP1, CHEK1, CHEK2, NBN, PALB2 and SLX4 variants.

Target cells of the disclosure may contain a sequence encoding one or more variant proteins including, but not limited to, BRCA1, BRCA2, RAD51, RAD51C, RAD51D, XRCC2, XPF, MRE11A, ATM, BARD1, BRIP1, CHEK1, CHEK2, NBN, PALB2 and SLX4 variants. In certain embodiments, the sequence encoding the variant protein comprises a promoter sequence and coding sequence, e.g., wherein the promoter sequence is operative linked to the coding sequence.

Target cells of the disclosure may contain a promoter sequence controlling the expression of one or more wild type or variant proteins including, but not limited to, BRCA1, BRCA2, RAD51, RAD51C, RAD51D, XRCC2, XPF, MRE11A, ATM, BARD1, BRIP1, CHEK1, CHEK2, NBN, PALB2 and SLX4. In some embodiments, the promoter sequence is silenced. Alternatively or in addition, in some embodiments, the promoter sequence comprises a mutation that disrupts a function or an activity of the promoter. Alternatively or in addition, in some embodiments, the promoter sequence comprises a mutation that induce silencing of the promoter.

Compositions and methods of the disclosure may inhibit a component of the same pathway in which a variant protein signals. In some embodiments, the variant protein comprises one or more of BRCA1, BRCA2, RAD51, RAD51C, RAD51D, XRCC2, XPF, MRE11A, ATM, BARD1, BRIP1, CHEK1, CHEK2, NBN, PALB2 and SLX4.

Compositions and methods of the disclosure may inhibit a component of the distinct pathway from which a variant protein signals. In some embodiments, the variant protein comprises one or more of BRCA1, BRCA2, RAD51, RAD51C, RAD51D, XRCC2, XPF, MRE11A, ATM, BARD1, BRIP1, CHEK1, CHEK2, NBN, PALB2 and SLX4.

Target cells of the disclosure may comprise a modification, such as a gene deletion or mutation, that results in no expression or reduced expression of BRCA1. Target cells of the disclosure may comprise a modification, such as an insertion, deletion, or substitution in the gene encoding BRCA1 or the encoded BRCA1 protein.

Target cells of the disclosure may express a BRCA1 protein according to SEQ ID NO: 13-20. Target cells of the disclosure may express a variant BRCA1 having at least 50%, 55%, 60%, 65%, 70%, 75%, 80%, 85%, 90%, 95%, 97%, 99% or any percentage in between of identity to SEQ ID NOs: 13-20.

In some embodiments of the compositions and methods of the disclosure, a BRCA1 protein of the disclosure comprises or consists of the amino acid sequence of (UniProt Accession No. P38398-1 and SEQ ID NO: 13):

| | |
|---|---|
| 1 | MDLSALRVEE VQNVINAMQK ILECPICLEL IKEPVSTKCD HIFCKFCMLK LLNQKKGPSQ |
| 61 | CPLCKNDITK RSLQESTRES QLVEELLKII CAFQLDTGLE YANSYNFAKK ENNSPEHLKD |
| 121 | EVSIIQSMGY RNRAKRLLQS EPENPSLQET SLSVQLSNLG TVRTLRTKQR IQPQKTSVYI |
| 181 | ELGSDSSEDT VNKATYCSVG DQELLQITPQ GTRDEISLDS AKKAACEFSE TDVTNTEHHQ |
| 241 | PSNNDLNTTE KRAAERHPEK YQGSSVSNLH VEPCGTNTHA SSLQHENSSL LLTKDRMNVE |
| 301 | KAEFCNKSKQ PGLARSQHNR WAGSKETCND RRTPSTEKKV DLNADPLCER KEWNKQKLPC |
| 361 | SENPRDTEDV PWITLNSSIQ KVNEWFSRSD ELLGSDDSHD GESESNAKVA DVLDVLNEVD |
| 421 | EYSGSSEKID LLASDPHEAL ICKSERVHSK SVESNIEDKI FGKTYRKKAS LPNLSHVTEN |
| 481 | LIIGAFVTEP QIIQERPLIN KLKRKRRPTS GLHPEDFIKK ADLAVQKTPE MINQGTNQTE |
| 541 | QNGQVMNITN SCHENKTKGD SIQNEKNPNP IESLEKESAF KTKAEPISSS ISNMELELNI |
| 601 | HNSKAPKKNR LRRKSSTRHI HALELVVSRN LSPPNCTELQ IDSCSSSEEI KKKKYNQMPV |
| 661 | RHSRNLQLME GKEPATGAKK SNKPNEQTSK RHDSDTFPEL KLTNAPGSFT KCSNTSELKE |
| 721 | FVNPSLPREE KEEKLETVKV SNNAEDPKDL MLSGERVLQT ERSVESSSIS LVPGTDYGTQ |
| 781 | ESISLLEVST LGKAKTEPNK CVSQCAAFEN PKGLIHGCSK DNRNDTEGFK YPLGHEVNHS |
| 841 | RETSIEMEES ELDAQYLQNT FKVSKRQSFA PFSNPGNAEE ECATFSAHSG SLKKQSPKVT |
| 901 | FECEQKEENQ GKNESNIKPV QTVNITAGFP VVGQKDKPVD NAKCSIKGGS RFCLSSQFRG |
| 961 | NETGLITPNK HGLLQNPYRI PPLFPIKSFV KTKCKKNLLE ENFEEHSMSP EREMGNENIP |
| 1021 | STVSTISRNN IRENVFKEAS SSNINEVGSS TNEVGSSINE IGSSDENIQA ELGRNRGPKL |
| 1081 | NAMLRLGVLQ PEVYKQSLPG SNCKHPEIKK QEYEEVVQTV NTDFSPYLIS DNLEQPMGSS |
| 1141 | HASQVCSETP DDLLDDGEIK EDTSFAENDI KESSAVESKS VQKGELSRSP SPFTHTHLAQ |
| 1201 | GYRRGAKKLE SSEENLSSED EELPCFQHLL FGKVNNIPSQ STRHSTVATE CLSKNTEENL |
| 1261 | LSLKNSLNDC SNQVILAKAS QEHHLSEETK CSASLFSSQC SELEDLTANT NTQDPFLIGS |
| 1321 | SKQMRHQSES QGVGLSDKEL VSDDEERGTG LEENNQEEQS MDSNLGEAAS GCESETSVSE |
| 1381 | DCSGLSSQSD ILTTQQRDTM QHNLIKLQQE MAELEAVLEQ HGSQPSNSYP SIISDSSALE |
| 1441 | DLRNPEQSTS EKAVLTSQKS SEYPISQNPE GLSADKFEVS ADSSTSKNKE PGVERSSPSK |
| 1501 | CPSLDDRWYM HSCSGSLQNR NYPSQEELIK VVDVEEQQLE ESGPHDLTET SYLPRQDLEG |
| 1561 | TPYLESGISL FSDDPESDPS EDRAPESARV GNIPSSTSAL KVPQLKVAES AQSPAAAHTT |
| 1621 | DTAGYNAMEE SVSREKPELT ASTERVNKRM SMVVSGLTPE EFMLVYKFAR KHHITLTNLI |
| 1681 | TEETTHVVMK TDAEFVCERT LKYFLGIAGG KWVVSYFWVT QSIKERKMLN EHDFEVRGDV |
| 1741 | VNGRNHQGPK RARESQDRKI FRGLEICCYG PFTNMPTDQL EWMVQLCGAS VVKELSSFTL |
| 1861 | GTGVHPIVVV QPDAWTEDNG FHAIGQMCEA PVVTREWVLD SVALYQCQEL DTYLIPQIPH |
| 1801 | SHY. |

In some embodiments of the compositions and methods of the disclosure, a BRCA1 protein of the disclosure comprises or consists of the amino acid sequence of (UniProt Accession No. P38398-2 and SEQ ID NO: 14):

| | |
|---|---|
| 1 | MDLSALRVEE VQNVINAMQK ILECPICLEL IKEPVSTKCD HIFCKFCMLK LLNQKKGPSQ |
| 61 | CPL. |

In some embodiments of the compositions and methods of the disclosure, a BRCA1 protein of the disclosure comprises or consists of the amino acid sequence of (UniProt Accession No. P38398-3 and SEQ ID NO: 15):

| | |
|---|---|
| 1 | MDLSALRVEE VQNVINAMQK ILECPICLEL IKEPVSTKCD HIFCKFCMLK LLNQKKGPSQ |
| 61 | CPLCKNDITK RSLQESTRFS QLVEELLKII CAFQLDTGLE YANSYNFAKK ENNSPEHLKD |
| 121 | EVSIIQSMGY RNRAKRLLQS EPENPSLQET SLSVQLSNLG TVRTLRTKQR IQPQKTSVYI |
| 181 | ELGSDSSEDT VNKATYCSVG DQELLQITPQ GTRDEISLDS AKKAACEFSE TDVTNTEHHQ |
| 241 | PSNNDLNTTE KRAAERHPEK YQGEAASGCE SETSVSEDCS GLSSQSDILT TQQRDTMQHN |
| 301 | LIKLQQEMAE LEAVLEQHGS QPSNSYPSII SDSSALEDLR NPEQSTSEKV LTSQKSSEYP |
| 361 | ISQNPEGLSA DKFEVSADSS TSKNKEPGVE RSSPSKCPSL DDRWYMHSCS GSLQNRNYPS |
| 421 | QEELIKVVDV EEQQLEESGP HDLTETSYLP RQDLEGTPYL ESGISLFSDD PESDPSEDRA |
| 481 | PESARVGNIP SSTSALKVPQ LKVAESAQSP AAAHTTDTAG YNAMEESVSR EKPELTASTE |
| 541 | RVNKRMSMVV SGLTPEEFML VYKFARKHHI TLTNLITEET THVVMKTDAE FVCERTLKYF |
| 601 | LGIAGGKWVV SYFWVTQSIK ERKMLNEHDF EVRGDVVNGR NHQGPKRARE SQDRKIFRGL |
| 661 | EICCYGPFTN MPTDQLEWMV QLCGASVVKE LSSFTLGTGV HPIVVVQPDA WTEDNGFHAI |
| 721 | GQMCEAPVVT REWVLDSVAL YQCQELDTYL IPQIPHSHY. |

In some embodiments of the compositions and methods of the disclosure, a BRCA1 protein of the disclosure comprises or consists of the amino acid sequence of (UniProt Accession No. P38398-4 and SEQ ID NO: 16):

| | |
|---|---|
| 1 | MQKILECPIC LELIKEPVST KCDHIFCKFC MLKLLNQKKG PSQCPLCKND ITKRSLQEST |
| 61 | RFSQLVEELL KIICAFQLDT GLEYANSYNF AKKENNSPEH LKDEVSIIQS MGYRNRAKRL |
| 121 | LQSEPENPSL QETSLSVQLS NLGTVRTLRT KQRIQPQKTS VYIELGSDSS EDTVNKATYC |
| 181 | SVGDQELLQI TPQGTRDEIS LDSAKKAACE FSETDVTNTE HHQPSNNDLN TTEKRAAERH |
| 241 | PEKYQGSSVS NLHVEPCGTN THASSLQHEN SSLLLTKDRM NVEKAEFCNK SKQPGLARSQ |
| 301 | HNRWAGSKET CNDRRTPSTE KKVDLNADPL CERKEWNKQK LPCSENPRDT EDVPWITLNS |
| 361 | SIQKVNEWFS RSDELLGSDD SHDGESESNA KVADVLDVLN EVDEYSGSSE KIDLLASDPH |
| 421 | EALICKSERV HSKSVESNIE DKIFGKTYRK KASLPNLSHV TENLIIGAFV TEPQIIQERP |
| 481 | LTNKLKRKRR PTSGLHPEDF IKKADLAVQK TPEMINQGTN QTEQNGQVMN ITNSGHENKT |
| 541 | KGDSIQNEKN PNPIESLEKE SAFKTKAEPI SSSISNMELE LNIHNSKAPK KNRLRRKSST |
| 601 | RHIHALELVV SRNLSPPNCT ELQIDSCSSS EEIKKKKYNQ MPVRHSRNLQ LMEGKEPATG |
| 661 | AKKSNKPNEQ TSKRHDSDTF PELKLTNAPG SFTKCSNTSE LKEFVNPSLP REEKEEKLET |
| 721 | VKVSNNAEDP KDLMLSGERV LQTERSVESS SISLVPGTDY GTQESISLLE VSTLGKAKTE |
| 781 | PNKCVSQCAA FENPKGLIHG CSKDNRNDTE GFKYPLGHEV NHSRETSIEM EESELDAQYL |
| 841 | QNTFKVSKRQ SFAPFSNPGN AEEECATFSA HSGSLKKQSP KVTFECEQKE ENQGKNESNI |
| 901 | KPVQTVNITA GFPVVGQKDK PVDNAKCSIK GGSRFCLSSQ FRGNETGLIT PNKHGLLQNP |
| 961 | YRIPPLFPIK SFVKTKCKKN LLEENFEEHS MSPEREMGNE NIPSTVSTIS RNNIRENVFK |
| 1021 | EASSSNINEV GSSTNEVGSS INEIGSSDEN IQAELGRNRG PKLNAMLRLG VLQPEVYKQS |
| 1081 | LPGSNCKHPE IKKQEYEEVV QTVNTDFSPY LISDNLEQPM GSSHASQVCS ETPDDLLDDG |
| 1141 | EIKEDTSFAE NDIKESSAVF SKSVQKGELS RSPSPFTHTH LAQGYRRGAK KLESSEENLS |
| 1201 | SEDEELPCFQ HLLFGKVNNI PSQSTRHSTV ATECLSKNTE ENLLSLKNSL NDCSNQVILA |
| 1261 | KASQEHHLSE ETKCSASLFS SQCSELEDLT ANTNTQDPFL IGSSKQMRHQ SESQGVGLSD |
| 1321 | KELVSDDEER GTGLEENNQE EQSMDSNLGE AASGCESETS VSEDCSGLSS QSDILTTQQR |
| 1381 | DTMQHNLIKL QQEMAELEAV LEQHGSQPSN SYPSIISDSS ALEDLRNPEQ STSEKAVLTS |
| 1441 | QKSSEYPISQ NPEGLSADKF EVSADSSTSK NKEPGVERSS PSKCPSLDDR WYMHSCSGSL |
| 1501 | QNRNYPSQEE LIKVVDVEEQ QLEESGPHDL TETSYLPRQD LEGTPYLESG ISLFSDDPES |
| 1561 | DPSEDRAPES ARVGNIPSST SALKVPQLKV AESAQSPAAA HTTDTAGYNA MEESVSREKP |
| 1621 | ELTASTERVN KRMSMVVSGL TPEEFMLVYK FARKHHITLT NLITEETTHV VMKTDAEFVC |
| 1681 | ERTLKYFLGI AGGKWVVSYF WVTQSIKERK MLNEHDFEVR GDVVNGRNHQ GPKRARESQD |
| 1741 | RKIFRGLEIC CYGPFTNMPT DQLEWMVQLC GASVVKELSS FTLGTGVHPI VVVQPDAWTE |
| 1801 | DNGFHAIGQM CEAPVVTREW VLDSVALYQC QELDTYLIPQ IPHSHY. |

In some embodiments of the compositions and methods of the disclosure, a BRCA1 protein of the disclosure comprises or consists of the amino acid sequence of (UniProt Accession No. P38398-5 and SEQ ID NO: 17):

| | |
|---|---|
| 1 | MDLSALRVEE VQNVINAMQK ILECPICLEL IKEPVSTKCD HIFCKFCMLK LLNQKKGPSQ |
| 61 | CPLCKNDITK RSLQESTRFS QLVEELLKII CAFQLDTGLE YANSYNFAKK ENNSPEHLKD |
| 121 | EVSIIQSMGY RNRAKRLLQS EPENPSLQET SLSVQLSNLG TVRTLRTKQR IQPQKTSVYI |
| 181 | ELGSDSSEDT VNKATYCSVG DQELLQITPQ GTRDEISLDS AKKGEAASGC ESETSVSEDC |
| 241 | SGLSSQSDIL TTQQRDTMQH NLIKLQQEMA ELEAVLEQHG SQPSNSYPSI ISDSSALEDL |
| 301 | RNPEQSTSEK AVLTSQKSSE YPISQNPEGL SADKFEVSAD SSTSKNKEPG VERSSPSKCP |
| 361 | SLDDRWYMHS CSGSLQNRNY PSQEELIKVV DVEEQQLEES GPHDLTETSY LPRQDLEGTP |
| 421 | YLESGISLES DDPESDPSED RAPESARVGN IPSSTSALKV PQLKVAESAQ SPAAAHTTDT |
| 481 | AGYNAMEESV SREKPELTAS TERVNKRMSM VVSGLTPEEF MLVYKFARKH HITLTNLITE |
| 541 | ETTHVVMKTD AEFVCERTLK YFLGIAGGKW VVSYFWVTQS IKERKMLNEH DFEVRGDVVN |
| 601 | GRNHQGPKRA RESQDRKIFR GLEICCYGPF TNMPTDQLEW MVQLCGASVV KELSSFTLGT |
| 721 | GVHPIVVVQP DAWTEDNGFH AIGQMCEAPV VTREWVLDSV ALYQCQELDT YLIPQIPHSH |
| 661 | Y. |

In some embodiments of the compositions and methods of the disclosure, a BRCA1 protein of the disclosure comprises or consists of the amino acid sequence of (UniProt Accession No. P38398-6 and SEQ ID NO: 18):

| | |
|---|---|
| 1 | MDLSALRVEE VQNVINAMQK ILECPICLEL IKEPVSTKCD HIFCKFCMLK LLNQKKGPSQ |
| 61 | CPLCKNDITK RSLQESTRFS QLVEELLKII CAFQLDTGLE YANSYNFAKK ENNSPEHLKD |
| 121 | EVSIIQSMGY RNRAKRLLQS EPENPSLQET SLSVQLSNLG TVRTLRTKQR IQPQKTSVYI |
| 181 | ELGSDSSEDT VNKATYCSVG DQELLQITPQ GTRDEISLDS AKKAACEFSE TDVTNTEHHQ |
| 241 | PSNNDLNTTE KRAAERHPEK YQGEAASGCE SETSVSEDCS GLSSQSDILT TQQRDTMQHN |
| 301 | LIKLQQEMAE LEAVLEQHGS QPSNSYPSII SDSSALEDLR NPEQSTSEKV LTSQKSSEYP |
| 361 | ISQNPEGLSA DKFEVSADSS TSKNKEPGVE RSSPSKCPSL DDRWYMHSCS GSLQNRNYPS |
| 421 | QEELIKVVDV EEQQLEESGP HDLTETSYLP RQDLEGTPYL ESGISLFSDD PESDPSEDRA |
| 481 | PESARVGNIP SSTSALKVPQ LKVAESAQSP AAAHTTDTAG YNAMEESVSR EKPELTASTE |
| 541 | RVNKRMSMVV SGLTPEEFML VYKFARKHHI TLTNLITEET THVVMKTDAE FVCERTLKYF |
| 601 | LGIAGGKWVV SYFWVTQSIK ERKMLNEHDF EVRGDVVNGR NHQGPKRARE SQDRKIFRGL |
| 661 | EICCYGPFTN MPTGCPPNCG CAARCLDRGQ WLPCNWADV. |

In some embodiments of the compositions and methods of the disclosure, a BRCA1 protein of the disclosure comprises or consists of the amino acid sequence of (UniProt Accession No. P38398-7 and SEQ ID NO: 19):

| | |
|---|---|
| 1 | MDLSALRVEE VQNVINAMQK ILECPICLEL IKEPVSTKCD HIFCKFCMLK LLNQKKGPSQ |
| 61 | CPLCKNDITK RSLQESTRES QLVEELLKII CAFQLDTGLE YANSYNFAKK ENNSPEHLKD |
| 121 | EVSIIQSMGY RNRAKRLLQS EPENPSLQET SLSVQLSNLG TVRTLRTKQR IQPQKTSVYI |
| 181 | ELGSDSSEDT VNKATYCSVG DQELLQITPQ GTRDEISLDS AKKAACEFSE TDVTNTEHHQ |
| 241 | PSNNDLNTTE KRAAERHPEK YQGSSVSNLH VEPCGTNTHA SSLQHENSSL LLTKDRMNVE |
| 301 | KAEFCNKSKQ PGLARSQHNR WAGSKETCND RRTPSTEKKV DLNADPLCER KEWNKQKLPC |
| 361 | SENPRDTEDV PWITLNSSIQ KVNEWFSRSD ELLGSDDSHD GESESNAKVA DVLDVLNEVD |
| 421 | EYSGSSEKID LLASDPHEAL ICKSERVHSK SVESNIEDKI FGKTYRKKAS LPNLSHVTEN |
| 481 | LIIGAFVTEP QIIQERPLIN KLKRKRRPTS GLHPEDFIKK ADLAVQKTPE MINQGTNQTE |
| 541 | QNGQVMNITN SGHENKTKGD SIQNEKNPNP IESLEKESAF KTKAEPISSS ISNMELELNI |
| 601 | HNSKAPKKNR LRRKSSTRHI HALELVVSRN LSPPNCTELQ IDSCSSSEEI KKKKYNQMPV |
| 661 | RHSRNLQLME GKEPATGAKK SNKPNEQTSK RHDSDTFPEL KLTNAPGSFT KCSNTSELKE |
| 721 | FVNPSLPREE KEEKLETVKV SNNAEDPKDL MLSGERVLQT ERSVESSSIS LVPGTDYGTQ |
| 781 | ESISLLEVST LGKAKTEPNK CVSQCAAFEN PKGLIHGCSK DNRNDTEGFK YPLGHEVNHS |
| 841 | RETSIEMEES ELDAQYLQNT FKVSKRQSFA PFSNPGNAEE ECATFSAHSG SLKKQSPKVT |
| 901 | FECEQKEENQ GKNESNIKPV QTVNITAGFP VVGQKDKPVD NAKCSIKGGS RFCLSSQFRG |
| 961 | NETGLITPNK HGLLQNPYRI PPLFPIKSFV KTKCKKNLLE ENFEEHSMSP EREMGNENIP |
| 1021 | STVSTISRNN IRENVFKEAS SSNINEVGSS TNEVGSSINE IGSSDENIQA ELGRNRGPKL |
| 1081 | NAMLRLGVLQ PEVYKQSLPG SNCKHPEIKK QEYEEVVQTV NTDFSPYLIS DNLEQPMGSS |
| 1141 | HASQVCSETP DDLLDDGEIK EDTSFAENDI KESSAVESKS VQKGELSRSP SPFTHTHLAQ |
| 1201 | GYRRGAKKLE SSEENLSSED EELPCFQHLL FGKVNNIPSQ STRHSTVATE CLSKNTEENL |
| 1261 | LSLKNSINDC SNQVILAKAS QEHHLSEETK CSASLFSSQC SELEDLTANT NTQDPFLIGS |
| 1321 | SKQMRHQSES QGVGLSDKEL VSDDEERGTG LEENNQEEQS MDSNLGEAAS GCESETSVSE |
| 1381 | DCSGLSSQSD ILTTQQRDTM QHNLIKLQQE MAELEAVLEQ HGSQPSNSYP SIISDSSALE |
| 1441 | DLRNPEQSTS EKDSHIHGQR NNSMFSKRPR EHISVLTSQK SSEYPISQNP EGLSADKFEV |
| 1501 | SADSSTSKNK EPGVERSSPS KCPSLDDRWY MHSCSGSLQN RNYPSQEELI KVVDVEEQQL |
| 1561 | EESGPHDLTE TSYLPRQDLE GTPYLESGIS LFSDDPESDP SEDRAPESAR VGNIPSSTSA |
| 1621 | LKVPQLKVAE SAQSPAAAHT TDTAGYNAME ESVSREKPEL TASTERVNKR MSMVVSGLTP |
| 1681 | EEFMLVYKFA RKHHITLTNL ITEETTHVVM KTDAEFVCER TLKYFLGIAG GKWVVSYFWV |
| 1741 | TQSIKERKML NEHDFEVRGD VVNGRNHQGP KRARESQDRK IFRGLEICCY GPFTNMPTDQ |
| 1801 | LEWMVQLCGA SVVKELSSFT LGTGVHPIVV VQPDAWTEDN GFHAIGQMCE APVVTREWVL |
| 1861 | DSVALYQCQE LDTYLIPQIP HSHY. |

In some embodiments of the compositions and methods of the disclosure, a BRCA1 protein of the disclosure comprises or consists of the amino acid sequence of (UniProt Accession No. P38398-8 and SEQ ID NO: 20):

| | |
|---|---|
| 1 | MLKLLNQKKG PSQCPLCKND ITKRSLQEST RFSQLVEELL KIICAFQLDT GLEYANSYNF |
| 61 | AKKENNSPEH LKDEVSIIQS MGYRNRAKRL LQSEPENPSL QETSLSVQLS NLGTVRTLRT |
| 121 | KQRIQPQKTS VYIELGSDSS EDTVNKATYC SVGDQELLQI TPQGTRDEIS LDSAKKAACE |
| 181 | FSETDVTNTE HHQPSNNDLN TTEKRAAERH PEKYQGSSVS NLHVEPCGTN THASSLQHEN |
| 241 | SSLLLTKDRM NVEKAEFCNK SKQPGLARSQ HNRWAGSKET CNDRRTPSTE KKVDLNADPL |
| 301 | CERKEWNKQK LPCSENPRDT EDVPWITLNS SIQKVNEWFS RSDELLGSDD SHDGESESNA |
| 361 | KVADVLDVLN EVDEYSGSSE KIDLLASDPH EALICKSERV HSKSVESNIE DKIFGKTYRK |
| 421 | KASLPNLSHV TENLIIGAFV TEPQIIQERP LTNKLKRKRR PTSGLHPEDF IKKADLAVQK |
| 481 | TPEMINQGTN QTEQNGQVMN ITNSGHENKT KGDSIQNEKN PNPIESLEKE SAFKTKAEPI |
| 541 | SSSISNMELE LNIHNSKAPK KNRLRRKSST RHIHALELVV SRNLSPPNCT ELQIDSCSSS |
| 601 | EEIKKKKYNQ MPVRHSRNLQ LMEGKEPATG AKKSNKPNEQ TSKRHDSDTF PELKLTNAPG |
| 661 | SFTKCSNTSE LKEFVNPSLP REEKEEKLET VKVSNNAEDP KDLMLSGERV LQTERSVESS |
| 721 | SISLVPGTDY GTQESISLLE VSTLGKAKTE PNKCVSQCAA FENPKGLIHG CSKDNRNDTE |
| 781 | GFKYPLGHEV NHSRETSIEM EESELDAQYL QNTFKVSKRQ SFAPFSNPGN AEEECATFSA |
| 841 | HSGSLKKQSP KVTFECEQKE ENQGKNESNI KPVQTVNITA GFPVVGQKDK PVDNAKCSIK |
| 901 | GGSRFCLSSQ FRGNETGLIT PNKHGLLQNP YRIPPLFPIK SFVKTKCKKN LLEENFEEHS |
| 961 | MSPEREMGNE NIPSTVSTIS RNNIRENVFK EASSSNINEV GSSTNEVGSS INEIGSSDEN |
| 1021 | IQAELGRNRG PKLNAMLRLG VLQPEVYKQS LPGSNCKHPE IKKQEYEEVV QTVNTDFSPY |
| 1081 | LISDNLEQPM GSSHASQVCS ETPDDLLDDG EIKEDTSFAE NDIKESSAVF SKSVQKGELS |
| 1141 | RSPSPFTHTH LAQGYRRGAK KLESSEENLS SEDEELPCFQ HLLFGKVNNI PSQSTRHSTV |
| 1201 | ATECLSKNTE ENLLSLKNSL NDCSNQVILA KASQEHHLSE ETKCSASLFS SQCSELEDLT |
| 1261 | ANTNTQDPFL IGSSKQMRHQ SESQGVGLSD KELVSDDEER GTGLEENNQE EQSMDSNLGE |
| 1321 | AASGCESETS VSEDCSGLSS QSDILTTQQR DTMQHNLIKL QQEMAELEAV LEQHGSQPSN |
| 1381 | SYPSIISDSS ALEDLRNPEQ STSEKAVLTS QKSSEYPISQ NPEGLSADKF EVSADSSTSK |
| 1441 | NKEPGVERSS PSKCPSLDDR WYMHSCSGSL QNRNYPSQEE LIKVVDVEEQ QLEESGPHDL |
| 1501 | TETSYLPRQD LEGTPYLESG ISLFSDDPES DPSEDRAPES ARVGNIPSST SALKVPQLKV |
| 1561 | AESAQSPAAA HTTDTAGYNA MEESVSREKP ELTASTERVN KRMSMVVSGL TPEEFMLVYK |
| 1621 | FARKHHITLT NLITEETTHV VMKTDAEFVC ERTLKYFLGI AGGKWVVSYF WVTQSIKERK |
| 1681 | MLNEHDFEVR GDVVNGRNHQ GPKRARESQD RKIFRGLEIC CYGPFTNMPT DQLEWMVQLC |
| 1801 | GASVVKELSS FTLGTGVHPI VVVQPDAWTE DNGFHAIGQM CEAPVVTREW VLDSVALYQC |
| 1741 | QELDTYLIPQ IPHSHY. |

Target cells of the disclosure may comprise a modification, such as a gene deletion or mutation, that results in no expression or reduced expression of BRCA2. Target cells of the disclosure may comprise a modification, such as an insertion, deletion, or substitution in the gene encoding BRCA2 or the encoded BRCA2 protein. Target cells of the disclosure may express a BRCA2 protein according to SEQ ID NO: 21. Target cells of the disclosure may express a variant BRCA2 having at least 50%, 55%, 60%, 65%, 70%, 75%, 80%, 85%, 90%, 95%, 97%, 99% or any percentage in between of identity to SEQ ID NO: 21.

In some embodiments of the compositions and methods of the disclosure, a BRCA2 protein of the disclosure comprises or consists of the amino acid sequence of (UniProt Accession No. P51587-1 and SEQ ID NO: 21):

| | |
|---|---|
| 1 | MPIGSKERPT FFEIFKTRCN KADLGPISLN WFEELSSEAP PYNSEPAEES EHKNNNYEPN |
| 61 | LFKTPQRKPS YNQLASTPII FKEQGLTLPL YQSPVKELDK FKLDLGRNVP NSRHKSLRTV |
| 121 | KTKMDQADDV SCPLLNSCLS ESPVVLQCTH VTPQRDKSVV CGSLFHTPKF VKGRQTPKHI |
| 181 | SESLGAEVDP DMSWSSSLAT PPTLSSTVLI VRNEEASETV FPHDTTANVK SYFSNHDESL |
| 241 | KKNDRFIASV TDSENTNQRE AASHGFGKTS GNSFKVNSCK DHIGKSMPNV LEDEVYETVV |
| 301 | DTSEEDSFSL CFSKCRTKNL QKVRTSKTRK KIFHEANADE CEKSKNQVKE KYSFVSEVEP |
| 361 | NDTDPLDSNV ANQKPFESGS DKISKEVVPS LACEWSQLTL SGLNGAQMEK IPLLHISSCD |
| 421 | QNISEKDLLD TENKRKKDFL TSENSLPRIS SLPKSEKPLN EETVVNKRDE EQHLESHTDC |
| 481 | ILAVKQAISG TSPVASSFQG IKKSIFRIRE SPKETFNASF SGHMTDPNFK KETEASESGL |
| 541 | EIHTVCSQKE DSLCPNLIDN GSWPATTTQN SVALKNAGLI STLKKKTNKF IYAIHDETSY |
| 601 | KGKKIPKDQK SELINCSAQF EANAFEAPLT FANADSGLLH SSVKRSCSQN DSEEPTLSLT |
| 661 | SSFGTILRKC SRNETCSNNT VISQDLDYKE AKCNKEKLQL FITPEADSLS CLQEGQCEND |
| 721 | PKSKKVSDIK EEVLAAACHP VQHSKVEYSD TDFQSQKSLL YDHENASTLI LTPTSKDVLS |
| 781 | NLVMISRGKE SYKMSDKLKG NNYESDVELT KNIPMEKNQD VCALNENYKN VELLPPEKYM |
| 841 | RVASPSRKVQ FNQNTNLRVI QKNQEETTSI SKITVNPDSE ELFSDNENNF VFQVANERNN |
| 901 | LALGNTKELH ETDLTCVNEP IFKNSTMVLY GDTGDKQATQ VSIKKDLVYV LAEENKNSVK |
| 961 | QHIKMTLGQD LKSDISLNID KIPEKNNDYM NKWAGLLGPI SNHSFGGSFR TASNKEIKLS |
| 1021 | EHNIKKSKMF FKDIEEQYPT SLACVEIVNT LALDNQKKLS KPQSINTVSA HLQSSVVVSD |
| 1081 | CKNSHITPQM LFSKQDFNSN HNLTPSQKAE ITELSTILEE SGSQFEFTQF RKPSYILQKS |
| 1141 | TFEVPENQMT ILKTTSEECR DADLHVIMNA PSIGQVDSSK QFEGTVEIKR KFAGLLKNDC |
| 1201 | NKSASGYLTD ENEVGFRGFY SAHGTKLNVS TEALQKAVKL FSDIENISEE TSAEVHPISL |
| 1261 | SSSKCHDSVV SMFKIENHND KTVSEKNNKC QLILQNNIEM TTGTFVEEIT ENYKRNTENE |
| 1321 | DNKYTAASRN SHNLEFDGSD SSKNDTVCIH KDETDLLFTD QHNICLKLSG QFMKEGNTQI |
| 1381 | KEDLSDLTFL EVAKAQEACH GNTSNKEQLT ATKTEQNIKD FETSDTFFQT ASGKNISVAK |
| 1441 | ESFNKIVNFF DQKPEELHNF SLNSELHSDI RKNKMDILSY EETDIVKHKI LKESVPVGTG |
| 1501 | NQLVTFQGQP ERDEKIKEPT LLGFHTASGK KVKIAKESLD KVKNLFDEKE QGTSEITSFS |
| 1561 | HQWAKTLKYR EACKDLELAC ETIEITAAPK CKEMQNSLNN DKNLVSIETV VPPKLLSDNL |
| 1621 | CRQTENLKTS KSIFLKVKVH ENVEKETAKS PATCYTNQSP YSVIENSALA FYTSCSRKTS |
| 1681 | VSQTSLLEAK KWLREGIFDG QPERINTADY VGNYLYENNS NSTIAENDKN HLSEKQDTYL |
| 1741 | SNSSMSNSYS YHSDEVYNDS GYLSKNKLDS GIEPVLKNVE DQKNTSFSKV ISNVKDANAY |
| 1801 | PQTVNEDICV EELVTSSSPC KNKNAAIKLS ISNSNNFEVG PPAFRIASGK IVCVSHETIK |
| 1861 | KVKDIFTDSF SKVIKENNEN KSKICQTKIM AGCYEALDDS EDILHNSLDN DECSTHSHKV |
| 1921 | FADIQSEEIL QHNQNMSGLE KVSKISPCDV SLETSDICKC SIGKLHKSVS SANTCGIFST |
| 1981 | ASGKSVQVSD ASLQNARQVF SEIEDSTKQV FSKVLFKSNE HSDQLTREEN TAIRTPEHLI |
| 2041 | SQKGFSYNVV NSSAFSGFST ASGKQVSILE SSLHKVKGVL EEFDLIRTEH SLHYSPTSRQ |
| 2101 | NVSKILPRVD KRNPEHCVNS EMEKTCSKEF KLSNNLNVEG GSSENNHSIK VSPYLSQFQQ |
| 2161 | DKQQLVLGTK VSLVENIHVL GKEQASPKNV KMEIGKTETF SDVPVKTNIE VCSTYSKDSE |
| 2221 | NYFETEAVEI AKAFMEDDEL TDSKLPSHAT HSLFTCPENE EMVLSNSRIG KRRGEPLILV |
| 2281 | GEPSIKRNLL NEFDRIIENQ EKSLKASKST PDGTIKDRRL FMHHVSLEPI TCVPFRTTKE |
| 2341 | RQEIQNPNFT APGQEFLSKS HLYEHLTLEK SSSNLAVSGH PFYQVSATRN EKMRHLITTG |
| 2401 | RPTKVFVPPF KTKSHFHRVE QCVRNINLEE NRQKQNIDGH GSDDSKNKIN DNEIHQFNKN |
| 2461 | NSNQAAAVTF TKCEEEPLDL ITSLQNARDI QDMRIKKKQR QRVFPQPGSL YLAKTSTLPR |
| 2521 | ISLKAAVGGQ VPSACSHKQL YTYGVSKHCI KINSKNAESF QFHTEDYFGK ESLWTGKGIQ |
| 2581 | LADGGWLIPS NDGKAGKEEF YRALCDTPGV DPKLISRIWV YNHYRWIIWK LAAMECAFPK |
| 2641 | EFANRCLSPE RVLLQLKYRY DTEIDRSRRS AIKKIMERDD TAAKTLVLCV SDIISLSANI |
| 2701 | SETSSNKTSS ADTQKVAIIE LTDGWYAVKA QLDPPLLAVL KNGRLTVGQK IILHGAELVG |
| 2761 | SPDACTPLEA PESLMLKISA NSTRPARWYT KLGFFPDPRP FPLPLSSLFS DGGNVGCVDV |
| 2821 | IIQRAYPIQW MEKTSSGLYI FRNEREEEKE AAKYVEAQQK RLEALFTKIQ EEFEEHEENT |
| 2881 | TKPYLPSRAL TRQQVRALQD GAELYEAVKN AADPAYLEGY FSEEQLRALN NHRQMLNDKK |
| 2941 | QAQIQLEIRK AMESAEQKEQ GLSRDVTTVW KLRIVSYSKK EKDSVILSIW RPSSDLYSLL |
| 3001 | TEGKRYRIYH LATSKSKSKS ERANIQLAAT KKTQYQQLPV SDEILFQIYQ PREPLHFSKF |
| 3061 | LDPDFQPSCS EVDLIGFVVS VVKKTGLAPF VYLSDECYNL LAIKFWIDLN EDIIKPHMLI |
| 3121 | AASNLQWRPE SKSGLLTLFA GDFSVFSASP KEGHFQETFN KMKNTVENID ILCNEAENKL |
| 3181 | MHILHANDPK WSTPTKDCTS GPYTAQIIPG TGNKLLMSSP NCEIYYQSPL SLCMAKRKSV |
| 3241 | STPVSAQMTS KSCKGEKEID DQKNCKKRRA LDFLSRLPLP PPVSPICTFV SPAAQKAFQP |
| 3301 | PRSCGTKYET PIKKKELNSP QMTPFKKFNE ISLLESNSIA DEELALINTQ ALLSGSTGEK |
| 3361 | QFISVSESTR TAPTSSEDYL RLKRRCTTSL IKEQESSQAS TEECEKNKQD TITTKKYI. |

In certain embodiments, a target cell comprises a BRCA gene with a mutation disclosed in Fatemeh Karami and Parvin Mehdipour, A Comprehensive Focus on Global Spectrum of BRCA1 and BRCA2 Mutations in Breast Cancer, Hindawi Publishing Corporation, BioMed Research International, Volume 2013, Article ID 928562, or Babita Sharma, MSc, Raman Preet Kaur, PhD, Sonali Raut, MSc, and Anjana Munshi, PhD, BRCA1 mutation spectrum, functions, and therapeutic strategies: The story so far, Current Problems in Cancer, 42, 2018, 189-207.

Target cells of the disclosure may comprise a modification, such as a gene deletion or mutation, that results in no expression or reduced expression of RAD51. Target cells of the disclosure may comprise a modification, such as an insertion, deletion, or substitution in the gene encoding RAD51 or the encoded RAD51 protein. Target cells of the disclosure may express a RAD51 protein according to SEQ ID NOs: 22-25. Target cells of the disclosure may express a variant RAD51 having at least 50%, 55%, 60%, 65%, 70%, 75%, 80%, 85%, 90%, 95%, 97%, 99% or any percentage in between of identity to SEQ ID NOs: 22-25.

In some embodiments of the compositions and methods of the disclosure, a RAD51 protein of the disclosure comprises or consists of the amino acid sequence of (UniProt Accession No. Q06609-1 and SEQ ID NO: 22):

| | |
|---|---|
| 1 | MAMQMQLEAN ADTSVEEESF GPQPISRLEQ EFHQRRSEII QITTGSKELD AVAYAPKKEL |
| 61 | INIKGISEAK ADKILAEAAK LVPMGFTTAT CGINANDVKK LEEAGFHTVE KLLQGGIETG |
| 121 | SITEMFGEFR TGKTQICHTL AVTCQLPIDR GGGEGKAMYI DTEGTFRPER LLAVAERYGL |
| 181 | SGSDVLDNVA YARAFNTDHQ TQLLYQASAM MVESRYALLI VDSATALYRT DYSGRGELSA |
| 241 | RQMHLARFLR MLLRLADEFG VAVVITNQVV AQVDGAAMFA ADPKKPIGGN IIAHASTTRL |
| 301 | YLRKGRGETR ICKIYDSPCL PEAEAMFAIN ADGVGDAKD. |

In some embodiments of the compositions and methods of the disclosure, a RAD51 protein of the disclosure comprises or consists of the amino acid sequence of (UniProt Accession No. Q06609-2 and SEQ ID NO: 23):

| | |
|---|---|
| 1 | MAMQMQLEAN ADTSVEEESF GPQPISRLEQ CGINANDVKK LEEAGFHTVE AVAYAPKKEL |
| 61 | INIKGISEAK ADKILAVAER YGLSGSDVLD NVAYARAFNT DHQTQLLYQA SAMMVESRYA |
| 121 | LLIVDSATAL YRTDYSGRGE LSARQMHLAR FLRMLLRLAD EFGVAVVITN QVVAQVDGAA |
| 181 | MFAADPKKPI GGNIIAHAST TRLYLRKGRG ETRICKIYDS PCLPEAEAMF AINADGVGDA |
| 241 | KD. |

In some embodiments of the compositions and methods of the disclosure, a RAD51 protein of the disclosure comprises or consists of the amino acid sequence of (UniProt Accession No. Q06609-3 and SEQ ID NO: 24):

| | |
|---|---|
| 1 | MAMQMQLEAN ADTSVEEESF GPQPISRLEQ CGINANDVKK LEEAGFHTVE AVAYAPKKEL |
| 61 | INIKGISEAK ADKILAEAAK LVPMGFTTAT EFHQRRSEII QITTGSKELD KLLQGGIETG |
| 121 | SITEMFGEFR TGKTQICHTL AVTCQLPIDR GGGEGKAMYI DTEGTFRPER LLAVAERYGL |
| 181 | SGSDVLDNVA YARAFNTDHQ TQLLYQASAM MVESRYALLI VDSATALYRT DYSGRGELSA |
| 241 | RQMHLARFLR MLLRLADEIV SEERKRGNQN LQNLRLSLSS. |

In some embodiments of the compositions and methods of the disclosure, a RAD51 protein of the disclosure comprises or consists of the amino acid sequence of (UniProt Accession No. Q06609-4 and SEQ ID NO: 25):

| | |
|---|---|
| 1 | MAMQMQLEAN ADTSVEEESF GPQPISRLEQ CGINANDVKK LEEAGFHTVE AVAYAPKKEL |
| 61 | INIKGISEAK ADKILTESRS VARLECNSVI LVYCTLRLSG SSDSPASASR VVGTTGGIET |
| 121 | GSITEMFGEF RTGKTQICHT LAVTCQLPID RGGGEGKAMY IDTEGTFRPE RLLAVAERYG |
| 181 | LSGSDVLDNV AYARAFNTDH QTQLLYQASA MMVESRYALL IVDSATALYR TDYSGRGELS |
| 241 | ARQMHLARFL RMLLRLADEF GVAVVITNQV VAQVDGAAMF AADPKKPIGG NIIAHASTTR |
| 301 | LYLRKGRGET RICKIYDSPC LPEAEAMFAI NADGVGDAKD. |

Target cells of the disclosure may comprise a modification, such as a gene deletion or mutation, that results in no expression or reduced expression of RAD51C. Target cells of the disclosure may comprise a modification, such as an insertion, deletion, or substitution in the gene encoding RAD51C or the encoded RAD51C protein. Target cells of the disclosure may express a RAD51C protein according to SEQ ID NOs: 26 or 27. Target cells of the disclosure may express a variant RAD51C having at least 50%, 55%, 60%, 65%, 70%, 75%, 80%, 85%, 90%, 95%, 97%, 99% or any percentage in between of identity to SEQ ID NOs: 26 or 27.

In some embodiments of the compositions and methods of the disclosure, a RAD51C protein of the disclosure comprises or consists of the amino acid sequence of (UniProt Accession No. 043502-1 and SEQ ID NO: 26):

| | |
|---|---|
| 1 | MRGKTFRFEM QRDLVSFPLS PAVRVKLVSA GFQTAEELLE VKPSELSKEV GISKAEALET |
| 61 | LQIIRRECLT NKPRYAGTSE SHKKCTALEL LEQEHTQGFI ITFCSALDDI LGGGVPLMKT |
| 121 | TEICGAPGVG KTQLCMQLAV DVQIPECFGG VAGEAVFIDT EGSFMVDRVV DLATACIQHL |
| 181 | QLIAEKHKGE EHRKALEDFT LDNILSHIYY FRCRDYTELL AQVYLLPDFL SEHSKVRLVI |
| 241 | VDGIAFPFRH DLDDLSLRTR LLNGLAQQMI SLANNHRLAV ILTNQMTTKI DRNQALLVPA |
| 301 | LGESWGHAAT IRLIFHWDRK QRLATLYKSP SQKECTVLFQ IKPQGFRDTV VTSACSLQTE |
| 361 | GSLSTRKRSR DPEEEL. |

In some embodiments of the compositions and methods of the disclosure, a RAD51C protein of the disclosure comprises or consists of the amino acid sequence of (UniProt Accession No. 043502-2 and SEQ ID NO: 27):

| | |
|---|---|
| 1 | MRGKTFRFEM QRDLVSFPLS PAVRVKLVSA GFQTAEELLE VKPSELSKEV GISKAEALET |
| 61 | LQIIRRECLT NKPRYAGTSE SHKKCTALEL LEQEHTQGFI ITFCSALDDI LGGGVPLMKT |
| 121 | TEICGAPGVG KTQLW. |

Target cells of the disclosure may express a RAD51D protein according to SEQ ID NOs: 28-35. Target cells of the disclosure may express a variant RAD51D having at least 50%, 55%, 60%, 65%, 70%, 75%, 80%, 85%, 90%, 95%, 97%, 99% or any percentage in between of identity to SEQ ID NOs: 28-35.

Target cells of the disclosure may comprise a modification, such as a gene deletion or mutation, that results in no expression or reduced expression of RAD51D. Target cells of the disclosure may comprise a modification, such as an insertion, deletion, or substitution in the gene encoding RAD51D or the encoded RAD51D protein. In some embodiments of the compositions and methods of the disclosure, a RAD51D protein of the disclosure comprises or consists of the amino acid sequence of (UniProt Accession No. 075771-1 and SEQ ID NO: 28):

| | |
|---|---|
| 1 | MGVLRVGLCP GLTEEMIQLL RSHRIKTVVD LVSADLEEVA QKCGLSYKAL VALRRVLLAQ |
| 61 | FSAFPVNGAD LYEELKTSTA ILSTGIGSLD KLLDAGLYTG EVTEIVGGPG SGKTQVCLCM |
| 121 | AANVAHGLQQ NVLYVDSNGG LTASRLLQLL QAKTQDEEEQ AEALRRIQVV HAFDIFQMLD |
| 181 | VLQELRGTVA QQVTGSSGTV KVVVVDSVTA VVSPLLGGQQ REGLALMMQL ARELKTLARD |
| 241 | LGMAVVVTNH ITRDRDSGRL KPALGRSWSF VPSTRILLDT IEGAGASGGR RMACLAKSSR |
| 301 | QPTGFQEMVD IGTWGTSEQS ATLQGDQT. |

In some embodiments of the compositions and methods of the disclosure, a RAD51D protein of the disclosure comprises or consists of the amino acid sequence of (UniProt Accession No. 075771-2 and SEQ ID NO: 29):

| | |
|---|---|
| 1 | MGVLRVGLCP GLTEEMIQLL RSHRIKTVVD LVSADLEEVA QKCGLSYKS. |

In some embodiments of the compositions and methods of the disclosure, a RAD51D protein of the disclosure comprises or consists of the amino acid sequence of (UniProt Accession No. 075771-3 and SEQ ID NO: 30):

| | |
|---|---|
| 1 | MGVLRVGLCP GLTEEMIQLL RSHRIKTVVD LVSADLEEVA QKCGLSYKAE ALRRIQVVHA |
| 61 | FDIFQMLDVL QELRGTVAQQ VTGSSGTVKV VVVDSVTAVV SPLLGGQQRE GLALMMQLAR |
| 121 | ELKTLARDLG MAVVVTNHIT RDRDSGRLKP ALGRSWSFVP STRILLDTIE GAGASGGRRM |
| 181 | ACLAKSSRQP TGFQEMVDIG TWGTSEQSAT LQGDQT. |

In some embodiments of the compositions and methods of the disclosure, a RAD51D protein of the disclosure comprises or consists of the amino acid sequence of (UniProt Accession No. 075771-4 and SEQ ID NO: 31):

| | |
|---|---|
| 1 | MGVLRVGLCP GLTEEMIQLL RSHRIKTVVD LVSADLEEVA QKCGLSYKAL VALRRVLLAQ |
| 61 | FSAFPVNGAD LYEELKTSTA ILSTGIGSLD KLLDAGLYTG EVTEIVGGPG SGKTQAEALR |
| 121 | RIQVVHAFDI FQMLDVLQEL RGTVAQQVTG SSGTVKVVVV DSVTAVVSPL LGGQQREGLA |
| 181 | LMMQLARELK TLARDLGMAV VVTNHITRDR DSGRLKPALG RSWSFVPSTR ILLDTIEGAG |
| 241 | ASGGRRMACL AKSSRQPTGF QEMVDIGTWG TSEQSATLQG DQT. |

In some embodiments of the compositions and methods of the disclosure, a RAD51D protein of the disclosure comprises or consists of the amino acid sequence of (UniProt Accession No. 075771-5 and SEQ ID NO: 32):

| | |
|---|---|
| 1 | MGVLRVGLCP GLTEEMIQLL RSHRIKTVVD LVSADLEEVA QKCGLSYKAL VALRRVLLAQ |
| 61 | FSAFPVNGAD LYEELKTSTA ILSTGIGRQK LSGGSRWCMH LVTEIVGGPG SGKTQVCLCM |
| 121 | AANVAHGLQQ NVLYVDSNGG LTASRLLQLL QAKTQDEEEQ AEALRRIQVV HAFDIFQMLD |
| 181 | VLQELRGTVA QQVTGSSGTV KVVVVDSVTA VVSPLLGGQQ REGLALMMQL ARELKTLARD |
| 241 | LGMAVVVTNH ITRDRDSGRL KPALGRSWSF VPSTRILLDT IEGAGASGGR RMACLAKSSR |
| 301 | QPTGFQEMVD IGTWGTSEQS ATLQGDQT. |

In some embodiments of the compositions and methods of the disclosure, a RAD51D protein of the disclosure comprises or consists of the amino acid sequence of (UniProt Accession No. 075771-6 and SEQ ID NO: 33):

| | |
|---|---|
| 1 | MGVLRVGLCP GLTEEMIQLL RSHRIKTVVD LVSADLEEVA QKCGLSYKAL VALRRVLLAQ |
| 61 | FSAFPVNGAD LYEELKTSTA ILSTGIGRHG GRTQVGTWED CSCLRSPQGD RGVGSGML. |

In some embodiments of the compositions and methods of the disclosure, a RAD51D protein of the disclosure comprises or consists of the amino acid sequence of (UniProt Accession No. 075771-7 and SEQ ID NO: 34):

| | |
|---|---|
| 1 | MGVLRVGLCP GLTEEMIQLL RSHRIKTVVD LVSADLEEVA QKCGLSYKAL VALRRVLLAQ |
| 61 | FSAFPVNGAD LYEELKTSTA ILSTGIGSLD KLLDAGLYTG EVTEIVGGPG SGKTQVCLCM |
| 121 | AANVAHGLQQ NVLYVDSNGG LTASRLLQLL QAKTQDEEEQ AEALRRIQVV HAFDIFQMLD |
| 181 | VLQELRGTVA QQDGIPEHLN HIPHCLHVHL PC. |

In some embodiments of the compositions and methods of the disclosure, a RAD51D protein of the disclosure comprises or consists of the amino acid sequence of (UniProt Accession No. 075771-8 and SEQ ID NO: 35):

| | |
|---|---|
| 1 | MGVLRVGLCP GLTEEMIQLL RSHRIKTVVD LVSADLEEVA QKCGLSYKTW RAHSSGNLGG |
| 61 | LQLPQVPAGR SWSGVRNALK KAGLGHGGTD GLSLNAFDER GTAVSTSRLD KLLDAGLYTG |
| 121 | EVTEIVGGPG SGKTQVCLCM AANVAHGLQQ NVLYVDSNGG LTASRLLQLL QAKTQDEEEQ |
| 181 | AEALRRIQVV HAFDIFQMLD VLQELRGTVA QQVTGSSGTV KVVVVDSVTA VVSPLLGGQQ |
| 241 | REGLALMMQL ARELKTLARD LGMAVVVTNH ITRDRDSGRL KPALGRSWSF VPSTRILLDT |
| 301 | IEGAGASGGR RMACLAKSSR QPTGFQEMVD IGTWGTSEQS ATLQGDQT. |

Target cells of the disclosure may comprise a modification, such as a gene deletion or mutation, that results in no expression or reduced expression of XRCC2. Target cells of the disclosure may comprise a modification, such as an insertion, deletion, or substitution in the gene encoding XRCC2 or the encoded XRCC2 protein. Target cells of the disclosure may express a XRCC2 protein according to SEQ ID NO: 36. Target cells of the disclosure may express a variant XRCC2 having at least 50%, 55%, 60%, 65%, 70%, 75%, 80%, 85%, 90%, 95%, 97%, 99% or any percentage in between of identity to SEQ ID NO: 36.

In some embodiments of the compositions and methods of the disclosure, a XRCC2 protein of the disclosure comprises or consists of the amino acid sequence of (UniProt Accession No. 043543-1 and SEQ ID NO: 36):

| | |
|---|---|
| 1 | MCSAFHRAES GTELLARLEG RSSLKEIEPN LFADEDSPVH GDILEFHGPE GTGKTEMLYH |
| 61 | LTARCILPKS EGGLEVEVLF IDTDYHFDML RLVTILEHRL SQSSEEIIKY CLGRFFLVYC |
| 121 | SSSTHLLLTL YSLESMFCSH PSLCLLILDS LSAFYWIDRV NGGESVNLQE STLRKCSQCL |
| 181 | EKLVNDYRLV LFATTQTIMQ KASSSSEEPS HASRRLCDVD IDYRPYLCKA WQQLVKHRMF |
| 241 | FSKQDDSQSS NQFSLVSRCL KSNSLKKHFF IIGESGVEFC. |

Target cells of the disclosure may comprise a modification, such as a gene deletion or mutation, that results in no expression or reduced expression of XPF. Target cells of the disclosure may comprise a modification, such as an insertion, deletion, or substitution in the gene encoding XPF or the encoded XPF protein. Target cells of the disclosure may express a XPF protein according to SEQ ID NO: 37 or 38. Target cells of the disclosure may express a variant XPF having at least 50%, 55%, 60%, 65%, 70%, 75%, 80%, 85%, 90%, 95%, 97%, 99% or any percentage in between of identity to SEQ ID NO: 37 or 38.

In some embodiments of the compositions and methods of the disclosure, a XPF protein of the disclosure comprises or consists of the amino acid sequence of (UniProt Accession No. Q92889-1 and SEQ ID NO: 37):

| | |
|---|---|
| 1 | MESGQPARRI AMAPLLEYER QLVLELLDTD GLVVCARGLG ADRLLYHFLQ LHCHPACLVL |
| 61 | VLNTQPAEEE YFINQLKIEG VEHLPRRVTN EITSNSRYEV YTQGGVIFAT SRILVVDFLT |
| 121 | DRIPSDLITG ILVYRAHRII ESCQEAFILR LFRQKNKRGF IKAFTDNAVA FDTGFCHVER |
| 181 | VMRNLFVRKL YLWPRFHVAV NSFLEQHKPE VVEIHVSMTP TMLAIQTAIL DILNACLKEL |
| 241 | KCHNPSLEVE DLSLENAIGK PFDKTIRHYL DPLWHQLGAK TKSLVQDLKI LRTLLQYLSQ |
| 301 | YDCVTFLNLL ESLRATEKAF GQNSGWLFLD SSTSMFINAR ARVYHLPDAK MSKKEKISEK |
| 361 | MEIKEGEETK KELVLESNPK WEALTEVLKE IEAENKESEA LGGPGQVLIC ASDDRTCSQL |
| 421 | RDYITLGAEA FLLRLYRKTF EKDSKAEEVW MKFRKEDSSK RIRKSHKRPK DPQNKERAST |
| 481 | KERTLKKKKR KLTLTQMVGK PEELEEEGDV EEGYRREISS SPESCPEEIK HEEFDVNLSS |
| 541 | DAAFGILKEP LTIIHPLLGC SDPYALTRVL HEVEPRYVVL YDAELTFVRQ LEIYRASRPG |
| 601 | KPLRVYFLIY GGSTEEQRYL TALRKEKEAF EKLIREKASM VVPEEREGRD ETNLDLVRGT |
| 661 | ASADVSTDTR KAGGQEQNGT QQSIVVDMRE FRSELPSLIH RRGIDIEPVT LEVGDYILTP |
| 721 | EMCVERKSIS DLIGSLNNGR LYSQCISMSR YYKRPVLLIE FDPSKPFSLT SRGALFQEIS |
| 781 | SNDISSKLTL LTLHFPRLRI LWCPSPHATA ELFEELKQSK PQPDAATALA ITADSETLPE |
| 841 | SEKYNPGPQD FLLKMPGVNA KNCRSLMHHV KNIAELAALS QDELTSILGN AANAKQLYDF |
| 901 | IHTSFAEVVS KGKGKK. |

In some embodiments of the compositions and methods of the disclosure, a XPF protein of the disclosure comprises or consists of the amino acid sequence of (UniProt Accession No. Q92889-2 and SEQ ID NO: 38):

| | |
|---|---|
| 1 | MESGQPARRI AMAPLLEYER QLVLELLDTD GLVVCARGLG |
| | ADRLLYHFLQ LHCHPACLVL |
| 61 | VLNTQPAEEE YFINQLKIEG VEHLPRRVTN EITSNSRYEV |
| | YTQGGVIFAT SRILVVDFLT |
| 121 | DRIPSDLITG ILVYRAHRII ESCQEAFILR LFRQKNKRGF |
| | IKAFTDNAVA FDTGFCHVER |
| 181 | VMRNLFVRKL YLWPRFHVAV NSFLEQHKPE VVEIHVSMTP |
| | TMLAIQTAIL DILNACLKEL |
| 241 | KCHNPSLEVE DLSLENAIGK PFDKTIRHYL DPLWHQLGAK |
| | TKSLVQDLKI LRTLLQYLSQ |
| 301 | YDCVTFLNLL ESLRATEKAF GQNSGWLFLD SSTSMFINAR |
| | ARVYHLPDAK MSKKEKISEK |
| 361 | MEIKEGEGIL WG. |

Target cells of the disclosure may comprise a modification, such as a gene deletion or mutation, that results in no expression or reduced expression of MRE11A. Target cells of the disclosure may comprise a modification, such as an insertion, deletion, or substitution in the gene encoding MRE11A or the encoded MRE11A protein. Target cells of the disclosure may express a MRE11A protein according to SEQ ID NO: 39-41. Target cells of the disclosure may express a variant MRE11A having at least 50%, 55%, 60%, 65%, 70%, 75%, 80%, 85%, 90%, 95%, 97%, 99% or any percentage in between of identity to SEQ ID NO: 39-41.

In some embodiments of the compositions and methods of the disclosure, a MRE11A protein of the disclosure comprises or consists of the amino acid sequence of (UniProt Accession No. P49959-1 and SEQ ID NO: 39):

| | |
|---|---|
| 1 | MSTADALDDE NTFKILVATD IHLGFMEKDA VRGNDTFVTL |
| | DEILRLAQEN EVDFILLGGD |
| 61 | LFHENKPSRK TLHTCLELLR KYCMGDRPVQ FEILSDQSVN |
| | FGFSKFPWVN YQDGNLNISI |
| 121 | PVFSIHGNHD DPTGADALCA LDILSCAGFV NHFGRSMSVE |
| | KIDISPVLLQ KGSTKIALYG |
| 181 | LGSIPDERLY RMFVNKKVTM LRPKEDENSW FNLFVIHQNR |
| | SKHGSTNFIP EQFLDDFIDL |
| 241 | VIWGHEHECK IAPTKNEQQL FYISQPGSSV VTSLSPGEAV |
| | KKHVGLLRIK GRKMNMHKIP |
| 301 | LHTVRQFFME DIVLANHPDI FNPDNPKVTQ AIQSFCLEKI |
| | EEMLENAERE RLGNSHQPEK |
| 361 | PLVRLRVDYS GGFEPFSVLR FSQKFVDRVA NPKDIIHFFR |
| | HREQKEKTGE EINFGKLITK |
| 421 | PSEGTTLRVE DLVKQYFQTA EKNVQLSLLT ERGMGEAVQE |
| | FVDKEEKDAI EELVKYQLEK |
| 481 | TQRFLKERHI DALEDKIDEE VRRFRETRQK NTNEEDDEVR |
| | EAMTRARALR SQSEESASAF |
| 541 | SADDLMSIDL AEQMANDSDD SISAATNKGR GRGRGRRGGR |
| | GQNSASRGGS QRGRADTGLE |
| 601 | TSTRSRNSKT AVSASRNMSI IDAFKSTRQQ PSRNVTTKNY |
| | SEVIEVDESD VEEDIFPTTS |
| 661 | KTDQRWSSTS SSKIMSQSQV SKGVDFESSE DDDDDPFMNT |
| | SSLRRNRR. |

In some embodiments of the compositions and methods of the disclosure, a MRE11A protein of the disclosure comprises or consists of the amino acid sequence of (UniProt Accession No. P49959-2 and SEQ ID NO: 40):

| | |
|---|---|
| 1 | MSTADALDDE NTFKILVATD IHLGFMEKDA VRGNDTFVTL |
| | DEILRLAQEN EVDFILLGGD |
| 61 | LFHENKPSRK TLHTCLELLR KYCMGDRPVQ FEILSDQSVN |
| | FGFSKFPWVN YQDGNLNISI |
| 121 | PVFSIHGNHD DPTGADALCA LDILSCAGFV NHFGRSMSVE |
| | KIDISPVLLQ KGSTKIALYG |
| 181 | LGSIPDERLY RMFVNKKVTM LRPKEDENSW FNLFVIHQNR |
| | SKHGSTNFIP EQFLDDFIDL |
| 241 | VIWGHEHECK IAPTKNEQQL FYISQPGSSV VTSLSPGEAV |
| | KKHVGLLRIK GRKMNMHKIP |
| 301 | LHTVRQFFME DIVLANHPDI FNPDNPKVTQ AIQSFCLEKI |
| | EEMLENAERE RLGNSHQPEK |
| 361 | PLVRLRVDYS GGFEPFSVLR FSQKFVDRVA NPKDIIHFFR |
| | HREQKEKTGE EINFGKLITK |
| 421 | PSEGTTLRVE DLVKQYFQTA EKNVQLSLLT ERGMGEAVQE |
| | FVDKEEKDAI EELVKYQLEK |
| 481 | TQRFLKERHI DALEDKIDEE VRRFRETRQK NTNEEDDEVR |
| | EAMTRARALR SQSEESASAF |
| 541 | SADDLMSIDL AEQMANDSDD SISAATNKGR GRGRGRRGGR |
| | GQNSASRGGS QRGRAFKSTR |
| 601 | QQPSRNVTTK NYSEVIEVDE SDVEEDIFPT TSKTDQRWSS |
| | TSSSKIMSQS QVSKGVDFES |
| 661 | SEDDDDDPFM NTSSLRRNRR. |

In some embodiments of the compositions and methods of the disclosure, a MRE11A protein of the disclosure comprises or consists of the amino acid sequence of (UniProt Accession No. P49959-3 and SEQ ID NO: 41):

| | |
|---|---|
| 1 | MNRNISHQKG DDENTFKILV ATDIHLGFME KDAVRGNDTF |
| | VTLDEILRLA QENEVDFILL |
| 61 | GGDLFHENKP SRKTLHTCLE LLRKYCMGDR PVQFEILSDQ |
| | SVNFGFSKFP WVNYQDGNLN |
| 121 | ISIPVFSIHG NHDDPTGADA LCALDILSCA GFVNHFGRSM |
| | SVEKIDISPV LLQKGSTKIA |
| 181 | LYGLGSIPDE RLYRMFVNKK VTMLRPKEDE NSWFNLFVIH |
| | QNRSKHGSTN FIPEQFLDDF |
| 241 | IDLVIWGHEH ECKIAPTKNE QQLFYISQPG SSVVTSLSPG |
| | EAVKKHVGLL RIKGRKMNMH |
| 301 | KIPLHTVRQF FMEDIVLANH PDIFNPDNPK VTQAIQSFCL |
| | EKIEEMLENA ERERLGNSHQ |
| 361 | PEKPLVRLRV DYSGGFEPFS VLRFSQKFVD RVANPKDIIH |
| | FFRHREQKEK TGEEINFGKL |
| 421 | ITKPSEGTTL RVEDLVKQYF QTAEKNVQLS LLTERGMGEA |
| | VQEFVDKEEK DAIEELVKYQ |
| 481 | LEKTQRFLKE RHIDALEDKI DEEVRRFRET RQKNTNEEDD |
| | EVREAMTRAR ALRSQSEESA |
| 541 | SAFSADDLMS IDLAEQMAND SDDSISAATN KGRGRGRGRR |
| | GGRGQNSASR GGSQRGRADT |
| 601 | GLETSTRSRN SKTAVSASRN MSIIDAFKST RQQPSRNVTT |
| | KNYSEVIEVD ESDVEEDIFP |
| 661 | TTSKTDQRWS STSSSKIMSQ SQVSKGVDFE SSEDDDDDPF |
| | MNTSSLRRNR R. |

Target cells of the disclosure may comprise a modification, such as a gene deletion or mutation, that results in no expression or reduced expression of ATM. Target cells of the disclosure may comprise a modification, such as an insertion, deletion, or substitution in the gene encoding ATM or the encoded ATM protein. Target cells of the disclosure may express an ATM protein according to SEQ ID NO: 42. Target cells of the disclosure may express a variant ATM having at least 50%, 55%, 60%, 65%, 70%, 75%, 80%, 85%, 90%, 95%, 97%, 99% or any percentage in between of identity to SEQ ID NO: 42.

In some embodiments of the compositions and methods of the disclosure, an ATM protein of the disclosure comprises or consists of the amino acid sequence of (UniProt Accession No. Q13315-1 and SEQ ID NO: 42):

| | |
|---|---|
| 1 | MSLVLNDLLI CCRQLEHDRA TERKKEVEKF KRLIRDPETI |
| | KHLDRHSDSK QGKYLNWDAV |
| 61 | FRFLQKYIQK ETECLRIAKP NVSASTQASR QKKMQEISSL |
| | VKYFIKCANR RAPRLKCQEL |
| 121 | LNYIMDTVKD SSNGAIYGAD CSNILLKDIL SVRKYWCEIS |
| | QQQWLELFSV YFRLYLKPSQ |
| 181 | DVHRVLVARI IHAVTKGCCS QTDGLNSKFL DFFSKAIQCA |
| | RQEKSSSGLN HILAALTIFL |
| 241 | KTLAVNFRIR VCELGDEILP TLLYIWTQHR LNDSLKEVII |
| | ELFQLQIYIH HPKGAKTQEK |
| 301 | GAYESTKWRS ILYNLYDLLV NEISHIGSRG KYSSGFRNIA |
| | VKENLIELMA DICHQVFNED |
| 361 | TRSLEISQSY TTTQRESSDY SVPCKRKKIE LGWEVIKDHL |
| | QKSQNDFDLV PWLQIATQLI |
| 421 | SKYPASLPNC ELSPLLMILS QLLPQQRHGE RTPYVLRCLT |
| | EVALCQDKRS NLESSQKSDL |
| 481 | LKLWNKIWCI TFRGISSEQI QAENFGLLGA IIQGSLVEVD |
| | REFWKLFTGS ACRPSCPAVC |
| 541 | CLTLALTTSI VPGTVKMGIE QNMCEVNRSF SLKESIMKWL |
| | LFYQLEGDLE NSTEVPPILH |
| 601 | SNFPHLVLEK ILVSLTMKNC KAAMNFFQSV PECEHHQKDK |
| | EELSFSEVEE LFLQTTFDKM |
| 661 | DFLTIVRECG IEKHQSSIGF SVHQNLKESL DRCLLGLSEQ |
| | LLNNYSSEIT NSETLVRCSR |
| 721 | LLVGVLGCYC YMGVIAEEEA YKSELFQKAK SLMQCAGESI |
| | TLFKNKTNEE FRIGSLRNMM |
| 781 | QLCTRCLSNC TKKSPNKIAS GFFLRLLTSK LMNDIADICK |
| | SLASFIKKPF DRGEVESMED |
| 841 | DTNGNLMEVE DQSSMNLFND YPDSSVSDAN EPGESQSTIG |
| | AINPLAEEYL SKQDLLFLDM |
| 901 | LKFLCLCVTT AQTNTVSFRA ADIRRKLLML IDSSTLEPTK |
| | SLHLHMYLML LKELPGEEYP |
| 961 | LPMEDVLELL KPLSNVCSLY RRDQDVCKTI LNHVLHVVKN |
| | LGQSNMDSEN TRDAQGQFLT |
| 1021 | VIGAFWHLTK ERKYIFSVRM ALVNCLKTLL EADPYSKWAI |
| | LNVMGKDFPV NEVFTQFLAD |
| 1081 | NHHQVRMLAA ESINRLFQDT KGDSSRLLKA LPLKLQQTAF |
| | ENAYLKAQEG MREMSHSAEN |
| 1141 | PETLDEIYNR KSVLLTLIAV VLSCSPICEK QALFALCKSV |
| | KENGLEPHLV KKVLEKVSET |
| 1201 | FGYRRLEDFM ASHLDYLVLE WLNLQDTEYN LSSFPFILLN |
| | YTNIEDFYRS CYKVLIPHLV |
| 1261 | IRSHFDEVKS IANQIQEDWK SLLTDCFPKI LVNILPYFAY |
| | EGTRDSGMAQ QRETATKVYD |
| 1321 | MLKSENLLGK QIDHLFISNL PEIVVELLMT LHEPANSSAS |
| | QSTDLCDFSG DLDPAPNPPH |
| 1381 | FPSHVIKATF AYISNCHKTK LKSILEILSK SPDSYQKILL |
| | AICEQAAETN NVYKKHRILK |
| 1441 | IYHLFVSLLL KDIKSGLGGA WAFVLRDVIY TLIHYINQRP |
| | SCIMDVSLRS FSLCCDLLSQ |
| 1501 | VCQTAVTYCK DALENHLHVI VGTLIPLVYE QVEVQKQVLD |
| | LLKYLVIDNK DNENLYITIK |
| 1561 | LLDPFPDHVV FKDLRITQQK IKYSRGPFSL LEEINHFLSV |
| | SVYDALPLTR LEGLKDLRRQ |
| 1621 | LELHKDQMVD IMRASQDNPQ DGIMVKLVVN LLQLSKMAIN |
| | HTGEKEVLEA VGSCLGEVGP |
| 1681 | IDFSTIAIQH SKDASYTKAL KLFEDKELQW TFIMLTYLNN |
| | TLVEDCVKVR SAAVTCLKNI |
| 1741 | LATKTGHSFW EIYKMTTDPM LAYLQPFRTS RKKFLEVPRF |
| | DKENPFEGLD DINLWIPLSE |
| 1801 | NHDIWIKTLT CAFLDSGGTK CEILQLLKPM CEVKTDFCQT |
| | VLPYLIHDIL LQDTNESWRN |
| 1861 | LLSTHVQGFF TSCLRHFSQT SRSTTPANLD SESEHFFRCC |
| | LDKKSQRTML AVVDYMRRQK |
| 1921 | RPSSGTIFND AFWLDLNYLE VAKVAQSCAA HFTALLYAEI |
| | YADKKSMDDQ EKRSLAFEEG |
| 1981 | SQSTTISSLS EKSKEETGIS LQDLLLEIYR SIGEPDSLYG |
| | CGGGKMLQPI TRLRTYEHEA |
| 2041 | MWGKALVTYD LETAIPSSTR QAGIIQALQN LGLCHILSVY |
| | LKGLDYENKD WCPELEELHY |
| 2101 | QAAWRNMQWD HCTSVSKEVE GTSYHESLYN ALQSLRDREF |
| | STFYESLKYA RVKEVEEMCK |
| 2161 | RSLESVYSLY PTLSRLQAIG ELESIGELFS RSVTHRQLSE |
| | VYIKWQKHSQ LLKDSDFSFQ |
| 2221 | EPIMALRTVI LEILMEKEMD NSQRECIKDI LTKHLVELSI |
| | LARTFKNTQL PERAIFQIKQ |
| 2281 | YNSVSCGVSE WQLEEAQVFW AKKEQSLALS ILKQMIKKLD |
| | ASCAANNPSL KLTYTECLRV |
| 2341 | CGNWLAETCL ENPAVIMQTY LEKAVEVAGN YDGESSDELR |
| | NGKMKAFLSL ARFSDTQYQR |
| 2401 | IENYMKSSEF ENKQALLKRA KEEVGLLREH KIQTNRYTVK |
| | VQRELELDEL ALRALKEDRK |
| 2461 | RFLCKAVENY INCLLSGEEH DMWVFRLCSL WLENSGVSEV |
| | NGMMKRDGMK IPTYKFLPLM |
| 2521 | YQLAARMGTK MMGGLGFHEV LNNLISRISM DHPHHTLFII |
| | LALANANRDE FLTKPEVARR |
| 2581 | SRITKNVPKQ SSQLDEDRTE AANRIICTIR SRRPQMVRSV |
| | EALCDAYIIL ANLDATQWKT |
| 2641 | QRKGINIPAD QPITKLKNLE DVVVPTMEIK VDHTGEYGNL |
| | VTIQSFKAEF RLAGGVNLPK |
| 2701 | IIDCVGSDGK ERRQLVKGRD DLRQDAVMQQ VFQMCNTLLQ |
| | RNTETRKRKL TICTYKVVPL |
| 2761 | SQRSGVLEWC TGTVPIGEFL VNNEDGAHKR YRPNDFSAFQ |
| | CQKKMMEVQK KSFEEKYEVF |
| 2821 | MDVCQNFQPV FRYFCMEKFL DPAIWFEKRL AYTRSVATSS |
| | IVGYILGLGD RHVQNILINE |
| 2881 | QSAELVHIDL GVAFEQGKIL PTPETVPFRL TRDIVDGMGI |
| | TGVEGVFRRC CEKTMEVMRN |
| 2941 | SQETLLTIVE VLLYDPLFDW TMNPLKALYL QQRPEDETEL |
| | HPTLNADDQE CKRNLSDIDQ |
| 3001 | SFNKVAERVL MRLQEKLKGV EEGTVLSVGG QVNLLIQQAI |
| | DPKNLSRLFP GWKAWV. |

Target cells of the disclosure may comprise a modification, such as a gene deletion or mutation, that results in no expression or reduced expression of BARD1. Target cells of the disclosure may comprise a modification, such as an insertion, deletion, or substitution in the gene encoding BARD1 or the encoded BARD1 protein. Target cells of the disclosure may express a BARD1 protein according to SEQ ID NOs 43-46. Target cells of the disclosure may express a variant BARD1 having at least 50%, 55%, 60%, 65%, 70%, 75%, 80%, 85%, 90%, 95%, 97%, 99% or any percentage in between of identity to SEQ ID NO: 43-46.

In some embodiments of the compositions and methods of the disclosure, a BARD1 protein of the disclosure comprises or consists of the amino acid sequence of (UniProt Accession No. Q99728-1 and SEQ ID NO: 43):

| | |
|---|---|
| 1 | MPDNRQPRNR QPRIRSGNEP RSAPAMEPDG RGAWAHSRAA |
| | LDRLEKLLRC SRCTNILREP |
| 61 | VCLGGCEHIF CSNCVSDCIG TGCPVCYTPA WIQDLKINRQ |
| | LDSMIQLCSK LRNLLHDNEL |
| 121 | SDLKEDKPRK SLFNDAGNKK NSIKMWFSPR SKKVRYVVSK |
| | ASVQTQPAIK KDASAQQDSY |
| 181 | EFVSPSPPAD VSERAKKASA RSGKKQKKKT LAEINQKWNL |
| | EAEKEDGEFD SKEESKQKLV |
| 241 | SFCSQPSVIS SPQINGEIDL LASGSLTESE CFGSLTEVSL |
| | PLAEQIESPD TKSRNEVVTP |
| 301 | EKVCKNYLTS KKSLPLENNG KRGHHNRLSS PISKRCRTSI |
| | LSTSGDFVKQ TVPSENIPLP |
| 361 | ECSSPPSCKR KVGGTSGRKN SNMSDEFISL SPGTPPSTLS |
| | SSSYRRVMSS PSAMKLLPNM |
| 421 | AVKRNHRGET LLHIASIKGD IPSVEYLLQN GSDPNVKDHA |
| | GWTPLHEACN HGHLKVVELL |
| 481 | LQHKALVNTT GYQNDSPLHD AAKNGHVDIV KLLLSYGASR |
| | NAVNIFGLRP VDYTDDESMK |
| 541 | SLLLLPEKNE SSSASHCSVM NTGQRRDGPL VLIGSGLSSE |
| | QQKMLSELAV ILKAKKYTEF |
| 601 | DSTVTHVVVP GDAVQSTLKC MLGILNGCWI LKFEWVKACL |
| | RRKVCEQEEK YEIPEGPRRS |
| 661 | RLNREQLLPK LFDGCYFYLW GTFKHHPKDN LIKLVTAGGG |
| | QILSRKPKPD SDVTQTINTV |
| 721 | AYHARPDSDQ RFCTQYIIYE DLCNYHPERV RQGKVWKAPS |
| | SWFIDCVMSF ELLPLDS. |

In some embodiments of the compositions and methods of the disclosure, a BARD1 protein of the disclosure comprises or consists of the amino acid sequence of (UniProt Accession No. Q99728-2 and SEQ ID NO: 44):

| | |
|---|---|
| 1 | MPDNRQPRNR QPRIRSGNEP RSAPAMEPDG RGAWAHSRAA |
| | LDRLEKLLRC SRCNCVSDCI |
| 61 | GTGCPVCYTP AWIQDLKINR QLDSMIQLCS KLRNLLHDNE |
| | LSDLKEDKPR KSLFNDAGNK |
| 121 | KNSIKMWFSP RSKKVRYVVS KASVQTQPAI KKDASAQQDS |
| | YEFVSPSPPA DVSERAKKAS |
| 181 | ARSGKKQKKK TLAEINQKWN LEAEKEDGEF DSKEESKQKL |
| | VSFCSQPSVI SSPQINGEID |
| 241 | LLASGSLTES ECFGSLTEVS LPLAEQIESP DTKSRNEVVT |
| | PEKVCKNYLT SKKSLPLENN |
| 301 | GKRGHHNRLS SPISKRCRTS ILSTSGDFVK QTVPSENIPL |
| | PECSSPPSCK RKVGGTSGRK |
| 361 | NSNMSDEFIS LSPGTPPSTL SSSSYRRVMS SPSAMKLLPN |
| | MAVKRNHRGE TLLHIASIKG |
| 421 | DIPSVEYLLQ NGSDPNVKDH AGWTPLHEAC NHGHLKVVEL |
| | LLQHKALVNT TGYQNDSPLH |
| 481 | DAAKNGHVDI VKLLLSYGAS RNAVNIFGLR PVDYTDDESM |
| | KSLLLLPEKN ESSSASHCSV |
| 541 | MNTGQRRDGP LVLIGSGLSS EQQKMLSELA VILKAKKYTE |
| | FDSTVTHVVV PGDAVQSTLK |
| 601 | CMLGILNGCW ILKFEWVKAC LRRKVCEQEE KYEIPEGPRR |
| | SRLNREQLLP KLFDGCYFYL |
| 661 | WGTFKHHPKD NLIKLVTAGG GQILSRKPKP DSDVTQTINT |
| | VAYHARPDSD QRECTQYIIY |
| 721 | EDLCNYHPER VRQGKVWKAP SSWFIDCVMS FELLPLDS. |

In some embodiments of the compositions and methods of the disclosure, a BARD1 protein of the disclosure comprises or consists of the amino acid sequence of (UniProt Accession No. Q99728-3 and SEQ ID NO: 45):

| | |
|---|---|
| 1 | MVAVPGPTVA PRSTAWRSCC AARVDLKEDK PRKSLFNDAG |
| | NKKNSIKMWF SPRSKKVRYV |
| 61 | VSKASVQTQP AIKKDASAQQ DSYEFVSPSP PADVSERAKK |
| | ASARSGKKQK KKTLAEINQK |
| 121 | WNLEAEKEDG EFDSKEESKQ KLVSFCSQPS VISSPQINGE |
| | IDLLASGSLT ESECFGSLTE |
| 181 | VSLPLAEQIE SPDTKSRNEV VTPEKVCKNY LTSKKSLPLE |
| | NNGKRGHHNR LSSPISKRCR |
| 241 | TSILSTSGDF VKQTVPSENI PLPECSSPPS CKRKVGGTSG |
| | RKNSNMSDEF ISLSPGTPPS |
| 301 | TLSSSSYRRV MSSPSAMKLL PNMAVKRNHR GETLLHIASI |
| | KGDIPSVEYL LQNGSDPNVK |
| 361 | DHAGWTPLHE ACNHGHLKVV ELLLQHKALV NTTGYQNDSP |
| | LHDAAKNGHV DIVKLLLSYG |
| 421 | ASRNAVNIFG LRPVDYTDDE SMKSLLLLPE KNESSSASHC |
| | SVMNTGQRRD GPLVLIGSGL |
| 481 | SSEQQKMLSE LAVILKAKKY TEFDSTVTHV VVPGDAVQST |
| | LKCMLGILNG CWILKFEWVK |
| 541 | ACLRRKVCEQ EEKYEIPEGP RRSRLNREQL LPKLFDGCYF |
| | YLWGTFKHHP KDNLIKLVTA |
| 601 | GGGQILSRKP KPDSDVTQTI NTVAYHARPD SDQRFCTQYI |
| | IYEDLCNYHP ERVRQGKVWK |
| 661 | APSSWFIDCV MSFELLPLDS. |

In some embodiments of the compositions and methods of the disclosure, a BARD1 protein of the disclosure comprises or consists of the amino acid sequence of (UniProt Accession No. Q99728-4 and SEQ ID NO: 46):

| | |
|---|---|
| 1 | MPDNRQPRNR QPRIRSGNEP RSAPAMEPDG RGAWAHSRAA |
| | LDRLEKLLRC SRCTNILREP |
| 61 | VCLGGCEHIF CSNCVSDCIG TGCPVCYTPA WIQDLKINRQ |
| | LDSMIQLCSK LRNLLHDNEL |
| 121 | SGRHTFC. |

Target cells of the disclosure may comprise a modification, such as a gene deletion or mutation, that results in no expression or reduced expression of BRIP1. Target cells of the disclosure may comprise a modification, such as an insertion, deletion, or substitution in the gene encoding BRIP1 or the encoded BRIP1 protein. Target cells of the disclosure may express a BRIP1 protein according to SEQ ID NOs 47-48. Target cells of the disclosure may express a variant BRIP1 having at least 50%, 55%, 60%, 65%, 70%, 75%, 80%, 85%, 90%, 95%, 97%, 99% or any percentage in between of identity to SEQ ID NO: 47-48.

In some embodiments of the compositions and methods of the disclosure, a BRIP1 protein of the disclosure comprises or consists of the amino acid sequence of (UniProt Accession No. Q9BX63-1 and SEQ ID NO: 47):

| | |
|---|---|
| 1 | MSSMWSEYTI GGVKIYFPYK AYPSQLAMMN SILRGLNSKQ |
| | HCLLESPTGS GKSLALLCSA |
| 61 | LAWQQSLSGK PADEGVSEKA EVQLSCCCAC HSKDFTNNDM |
| | NQGTSRHFNY PSTPPSERNG |
| 121 | TSSTCQDSPE KTTLAAKLSA KKQASIYRDE NDDFQVEKKR |
| | IRPLETTQQI RKRHCFGTEV |
| 181 | HNLDAKVDSG KTVKLNSPLE KINSFSPQKP PGHCSRCCCS |
| | TKQGNSQESS NTIKKDHTGK |
| 241 | SKIPKIYFGT RTHKQIAQIT RELRRTAYSG VPMTILSSRD |
| | HTCVHPEVVG NFNRNEKCME |
| 301 | LLDGKNGKSC YFYHGVHKIS DQHTLQTFQG MCKAWDIEEL |
| | VSLGKKLKAC PYYTARELIQ |
| 361 | DADIIFCPYN YLLDAQIRES MDLNLKEQVV ILDEAHNIED |
| | CARESASYSV TEVQLRFARD |
| 421 | ELDSMVNNNI RKKDHEPLRA VCCSLINWLE ANAEYLVERD |
| | YESACKIWSG NEMLLTLHKM |
| 481 | GITTATFPIL QGHFSAVLQK EEKISPIYGK EEAREVPVIS |
| | ASTQIMLKGL FMVLDYLFRQ |
| 541 | NSRFADDYKI AIQQTYSWTN QIDISDKNGL LVLPKNKKRS |
| | RQKTAVHVLN FWCLNPAVAF |
| 601 | SDINGKVQTI VLTSGTLSPM KSFSSELGVT FTIQLEANHI |
| | IKNSQVWVGT IGSGPKGRNL |
| 661 | CATFQNTETF EFQDEVGALL LSVCQTVSQG ILCFLPSYKL |
| | LEKLKERWLS TGLWHNLELV |
| 721 | KTVIVEPQGG EKTNFDELLQ VYYDAIKYKG EKDGALLVAV |
| | CRGKVSEGLD FSDDNARAVI |
| 781 | TIGIPFPNVK DLQVELKRQY NDHHSKLRGL LPGRQWYEIQ |
| | AYRALNQALG RCIRHRNDWG |
| 841 | ALILVDDRFR NNPSRYISGL SKWVRQQIQH HSTFESALES |
| | LAEFSKKHQK VLNVSIKDRT |
| 901 | NIQDNESTLE VTSLKYSTSP YLLEAASHLS PENFVEDEAK |
| | ICVQELQCPK IITKNSPLPS |
| 961 | SIISRKEKND PVFLEEAGKA EKIVISRSTS PTFNKQTKRV |
| | SWSSFNSLGQ YFTGKIPKAT |
| 1021 | PELGSSENSA SSPPRFKTEK MESKTVLPFT DKCESSNLTV |
| | NTSFGSCPQS ETIISSLKID |
| 1081 | ATLTRKNHSE HPLCSEEALD PDIELSLVSE EDKQSTSNRD |
| | FETEAEDESI YFTPELYDPE |
| 1141 | DTDEEKNDLA ETDRGNRLAN NSDCILAKDL FEIRTIKEVD |
| | SAREVKAEDC IDTKLNGILH |
| 1201 | IEESKIDDID GNVKTTWINE LELGKTHEIE IKNFKPSPSK |
| | NKGMFPGFK. |

In some embodiments of the compositions and methods of the disclosure, a BRIM protein of the disclosure comprises or consists of the amino acid sequence of (UniProt Accession No. Q9BX63-2 and SEQ ID NO: 48):

| | |
|---|---|
| 1 | MSSMWSEYTI GGVKIYFPYK AYPSQLAMMN SILRGLNSKQ |
| | HCLLESPTGS GKSLALLCSA |
| 61 | LAWQQSLSGK PADEGVSEKA EVQLSCCCAC HSKDFTNNDM |
| | NQGTSRHFNY PSTPPSERNG |
| 121 | TSSTCQDSPE KTTLAAKLSA KKQASIYRDE NDDFQVEKKR |
| | IRPLETTQQI RKRHCFGTEV |
| 181 | HNLDAKVDSG KTVKLNSPLE KINSFSPQKP PGHCSRCCCS |
| | TKQGNSQESS NTIKKDHTGK |
| 241 | SKIPKIYFGT RTHKQIAQIT RELRRTAYSG VPMTILSSRD |
| | HTCVHPEVVG NFNRNEKCME |
| 301 | LLDGKNGKSC YFYHGVHKIS DQHTLQTFQG MCKAWDIEEL |
| | VSLGKKLKAC PYYTARELIQ |
| 361 | DADIIFCPYN YLLDAQIRES MDLNLKEQVV ILDEAHNIED |
| | CARESASYSV TEVQLRFARD |
| 421 | ELDSMVNNNI RKKDHEPLRA VCCSLINWLE ANAEYLVERD |
| | YESACKIWSG NEMLLTLHKM |
| 481 | GITTATFPIL QGHFSAVLQK EEKISPIYGK EEAREVPVIS |
| | ASTQIMLKGL FMVLDYLFRQ |
| 541 | NSRFADDYKI AIQQTYSWTN QIDISDKNGL LVLPKNKKRS |
| | RQKTAVHVLN FWCLNPAVAF |
| 601 | SDINGKVQTI VLTSGTLSPM KSFSSELGVT FTIQLEANHI |
| | IKNSQVWVGT IGSGPKGRNL |
| 661 | CATFQNTETF EFQDEVGALL LSVCQTVSQG ILCFLPSYKL |
| | LEKLKERWLS TGLWHNLELV |
| 721 | KTVIVEPQGG EKTNFDELLQ VYYDAIKYKG EKDGALLVAV |
| | CRGKVSEGLD FSDDNARAVI |
| 781 | TIGIPFPNVK DLQVELKRQY NDHHSKLRGL LPGRQWYEIQ |
| | AYRALNQALG RCIRHRNDWG |
| 841 | ALILVDDRFR NNPSRYISGL SKWVRQQIQH HSTFESALES |
| | LAEFSKKHQK VLNVSIKDRT |
| 901 | NIQDNESTLE VTSLKYSTSP YLLEAASHLS PENFVEDEAK |
| | ICVQELQCPK IITKNSPLPS |
| 961 | SIISRKEKSM KSSSHLPLIE KSFIIFSEMI FIWV. |

Target cells of the disclosure may comprise a modification, such as a gene deletion or mutation, that results in no expression or reduced expression of CHEK1. Target cells of the disclosure may comprise a modification, such as an insertion, deletion, or substitution in the gene encoding CHEK1 or the encoded CHEK1 protein. Target cells of the disclosure may express a CHEK1 protein according to SEQ ID NOs 49-51. Target cells of the disclosure may express a variant CHEK1 having at least 50%, 55%, 60%, 65%, 70%, 75%, 80%, 85%, 90%, 95%, 97%, 99% or any percentage in between of identity to SEQ ID NO: 49-51.

In some embodiments of the compositions and methods of the disclosure, a CHEK1 protein of the disclosure comprises or consists of the amino acid sequence of (UniProt Accession No. 014757-1 and SEQ ID NO: 49):

| | |
|---|---|
| 1 | MAVPFVEDWD LVQTLGEGAY GEVQLAVNRV TEEAVAVKIV |
| | DMKRAVDCPE NIKKEICINK |
| 61 | MLNHENVVKF YGHRREGNIQ YLFLEYCSGG ELFDRIEPDI |
| | GMPEPDAQRF FHQLMAGVVY |
| 121 | LHGIGITHRD IKPENLLLDE RDNLKISDFG LATVFRYNNR |
| | ERLLNKMCGT LPYVAPELLK |
| 181 | RREFHAEPVD VWSCGIVLTA MLAGELPWDQ PSDSCQEYSD |
| | WKEKKTYLNP WKKIDSAPLA |
| 241 | LLHKILVENP SARITIPDIK KDRWYNKPLK KGAKRPRVTS |
| | GGVSESPSGF SKHIQSNLDF |
| 301 | SPVNSASSEE NVKYSSSQPE PRTGLSLWDT SPSYIDKLVQ |
| | GISFSQPTCP DHMLLNSQLL |
| 361 | GTPGSSQNPW QRLVKRMTRF FTKLDADKSY QCLKETCEKL |
| | GYQWKKSCMN QVTISTTDRR |
| 421 | NNKLIFKVNL LEMDDKILVD FRISKGDGLE FKRHFLKIKG |
| | KLIDIVSSQK IWLPAT. |

In some embodiments of the compositions and methods of the disclosure, a CHEK1 protein of the disclosure comprises or consists of the amino acid sequence of (UniProt Accession No. 014757-2 and SEQ ID NO: 50):

| | |
|---|---|
| 1 | MEKPDIGMPE PDAQRFFHQL MAGVVYLHGI GITHRDIKPE |
| | NLLLDERDNL KISDFGLATV |
| 61 | FRYNNRERLL NKMCGTLPYV APELLKRREF HAEPVDVWSC |
| | GIVLTAMLAG ELPWDQPSDS |
| 121 | CQEYSDWKEK KTYLNPWKKI DSAPLALLHK ILVENPSARI |
| | TIPDIKKDRW YNKPLKKGAK |
| 181 | RPRVTSGGVS ESPSGFSKHI QSNLDFSPVN SASSEENVKY |
| | SSSQPEPRTG LSLWDTSPSY |
| 241 | IDKLVQGISF SQPTCPDHML LNSQLLGTPG SSQNPWQRLV |
| | KRMTRFFTKL DADKSYQCLK |
| 301 | ETCEKLGYQW KKSCMNQVTI STTDRRNNKL IFKVNLLEMD |
| | DKILVDFRLS KGDGLEFKRH |
| 361 | FLKIKGKLID IVSSQKIWLP AT. |

In some embodiments of the compositions and methods of the disclosure, a CHEK1 protein of the disclosure comprises or consists of the amino acid sequence of (UniProt Accession No. 014757-3 and SEQ ID NO: 51):

| | |
|---|---|
| 1 | MAVPFVEDWD LVQTLGEGAY GEVQLAVNRV TEEAVAVKIV |
| | DMKRAVDCPE NIKKEICINK |
| 61 | MLNHENVVKF YGHRREGNIQ YLFLEYCSGG ELFDRIEPDI |
| | GMPEPDAQRF FHQLMAGVVY |
| 121 | LHGIGITHRD IKPENLLLDE RDNLKISDFG LATVFRYNNR |
| | ERLLNKMCGT LPYVAPELLK |
| 181 | RREFHAEPVD VWSCGIVLTA MLAGELPWDQ PSDSCQEYSD |
| | WKEKKTYLNP WKKIDSAPLA |
| 241 | LLHKILVENP SARITIPDIK KDRWYNKPLK KGAKRPRVTS |
| | GGVSESPSGF SKHIQSNLDF |
| 301 | SPVNSASSEE NVKYSSSQPE PRTGLSLWDT SPSYIDKLVQ |
| | GISFSQPTCP DHMLLNSQLL |
| 361 | GTPGSSQNPW QRLVKRMTRF FTKLDADKSY QCLKETCEKL |
| | GYQWKKSCMN QGDGLEFKRH |
| 421 | FLKIKGKLID IVSSQKIWLP AT. |

Target cells of the disclosure may comprise a modification, such as a gene deletion or mutation, that results in no expression or reduced expression of CHEK2. Target cells of the disclosure may comprise a modification, such as an insertion, deletion, or substitution in the gene encoding CHEK2 or the encoded CHEK2 protein. Target cells of the disclosure may express a CHEK2 protein according to SEQ ID NOs 52-64. Target cells of the disclosure may express a variant CHEK2 having at least 50%, 55%, 60%, 65%, 70%, 75%, 80%, 85%, 90%, 95%, 97%, 99% or any percentage in between of identity to SEQ ID NO: 52-64.

In some embodiments of the compositions and methods of the disclosure, a CHEK2 protein of the disclosure comprises or consists of the amino acid sequence of (UniProt Accession No. 096017-1 and SEQ ID NO: 52):

| | |
|---|---|
| 1 | MSRESDVEAQ QSHGSSACSQ PHGSVTQSQG SSSQSQGISS |
| | SSTSTMPNSS QSSHSSSGTL |
| 61 | SSLETVSTQE LYSIPEDQEP EDQEPEEPTP APWARLWALQ |
| | DGFANLECVN DNYWFGRDKS |
| 121 | CEYCFDEPLL KRTDKYRTYS KKHFRIFREV GPKNSYIAYI |
| | EDHSGNGTFV NTELVGKGKR |
| 181 | RPLNNNSEIA LSLSRNKVFV FFDLTVDDQS VYPKALRDEY |
| | IMSKTLGSGA CGEVKLAFER |
| 241 | KTCKKVAIKI ISKRKFAIGS AREADPALNV ETEIEILKKL |
| | NHPCIIKIKN FFDAEDYYIV |
| 301 | LELMEGGELF DKVVGNKRLK EATCKLYFYQ MLLAVQYLHE |
| | NGIIHRDLKP ENVLLSSQEE |
| 361 | DCLIKITDFG HSKILGETSL MRTLCGTPTY LAPEVLVSVG |
| | TAGYNRAVDC WSLGVILFIC |
| 421 | LSGYPPFSEH RTQVSLKDQI TSGKYNFIPE VWAEVSEKAL |
| | DLVKKLLVVD PKARFTTEEA |
| 481 | LRHPWLQDED MKRKFQDLLS EENESTALPQ VLAQPSTSRK |
| | RPREGEAEGA ETTKRPAVCA |
| 541 | AVL. |

In some embodiments of the compositions and methods of the disclosure, a CHEK2 protein of the disclosure comprises or consists of the amino acid sequence of (UniProt Accession No. 096017-2 and SEQ ID NO: 53):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL KRTDKYRTYS KKHFRIFREV GPKNSYIAYI EDHSGNGTFV NTELVGKGKR |
| 181 | RPLNNNSEIA LSLSRNKEKI LKIYSLSRFS KIRRGAVAHV FNPSTLGGRG WQIT. |

In some embodiments of the compositions and methods of the disclosure, a CHEK2 protein of the disclosure comprises or consists of the amino acid sequence of (UniProt Accession No. 096017-3 and SEQ ID NO: 54):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLDEDM KRKFQDLLSE |
| 121 | ENESTALPQV LAQPSTSRKR PREGEAEGAE TTKRPAVCAA VL. |

In some embodiments of the compositions and methods of the disclosure, a CHEK2 protein of the disclosure comprises or consists of the amino acid sequence of (UniProt Accession No. 096017-4 and SEQ ID NO: 55):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLVFVF FDLTVDDQSV |
| 121 | YPKALRDEYI MSKTLGSGAC GEVKLAFERK TCKKVAIKII SKRKFAIGSA READPALNVE |
| 181 | TEIEILKKLN HPCIIKIKNF FDAEDYYIVL ELMEGGELFD KVVGNKRLKE ATCKLYFYQM |
| 241 | LLAVQYLHEN GIIHRDLKPE NVLLSSQEED CLIKITDFGH SKILGETSLM RTLCGTPTYL |
| 301 | APEVLVSVGT AGYNRAVDCW SLGVILFICL SGYPPFSEHR TQVSLKDQIT SGKYNFIPEV |
| 361 | WAEVSEKALD LVKKLLVVDP KARFTTEEAL RHPWLQDEDM KRKFQDLLSE ENESTALPQV |
| 421 | LAQPSTSRKR PREGEAEGAE TTKRPAVCAA VL. |

In some embodiments of the compositions and methods of the disclosure, a CHEK2 protein of the disclosure comprises or consists of the amino acid sequence of (UniProt Accession No. 096017-5 and SEQ ID NO: 56):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL KRTDKYRTYS KKHFRIFREV GPKNSYIAYI EDHSGNGTFV NTELVGKGKR |
| 181 | RPLNNNSEIA LSLSRNKVVP VER. |

In some embodiments of the compositions and methods of the disclosure, a CHEK2 protein of the disclosure comprises or consists of the amino acid sequence of (UniProt Accession No. 096017-6 and SEQ ID NO: 57):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 12 | CEYCFDEPLL KRTDKYRTYS KKHFRIFREE NLSCPYRIWF NFCLF. |

In some embodiments of the compositions and methods of the disclosure, a CHEK2 protein of the disclosure comprises or consists of the amino acid sequence of (UniProt Accession No. 096017-7 and SEQ ID NO: 58):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL KRTDKYRTYS KKHFRIFREV GPKNSYIAYI EDHSGNGTFV NTELVGKGKR |
| 181 | RPLNNNSEIA LSLSRNKVFV FFDLTVDDQS VYPKALRDEY IMSKTLGSGA CGEVKLAFER |
| 241 | KTCKKVAIKI ISKRKFAIGS AREADPALNV ETEIEILKKL NHPCIIKIKN FFDAEDYYIV |
| 301 | LELMEGGELF DKVVGNKRLK EATCKLYFYQ MLLAVQMKT. |

In some embodiments of the compositions and methods of the disclosure, a CHEK2 protein of the disclosure comprises or consists of the amino acid sequence of (UniProt Accession No. 096017-8 and SEQ ID NO: 59):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL KRTDKYRTYS KKHFRIFREV GPKNSYIAYI EDHSGNGTFV NTELVGKGKR |
| 181 | RPLNNNSEIA LSLSRNKVFV FFDLTVDDQS VYPKALRDEY IMSKTLGSGA CGEVKLAFER |
| 241 | KTCKKVAIKI ISKRKFAIGS AREADPALNV ETEIEILKKL NHDGRGRAV. |

In some embodiments of the compositions and methods of the disclosure, a CHEK2 protein of the disclosure comprises or consists of the amino acid sequence of (UniProt Accession No. 096017-9 and SEQ ID NO: 60):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLETES GHVTQSDLEL |
| 121 | LLSSDPPASA SQSAGIRGVR HHPRPVCSLK CVNDNYWFGR DKSCEYCFDE PLLKRTDKYR |
| 181 | TYSKKHFRIF REVGPKNSYI AYIEDHSGNG TFVNTELVGK GKRRPLNNNS EIALSLSRNK |
| 241 | VFVFFDLTVD DQSVYPKALR DEYIMSKTLG SGACGEVKLA FERKTCKKVA IKIISKRKFA |
| 301 | IGSAREADPA LNVETEIEIL KKLNHPCIIK IKNFFDAEDY YIVLELMEGG ELFDKVVGNK |
| 361 | RLKEATCKLY FYQMLLAVQY LHENGIIHRD LKPENVLLSS QEEDCLIKIT DFGHSKILGE |
| 421 | TSLMRTLCGT PTYLAPEVLV SVGTAGYNRA VDCWSLGVIL FICLSGYPPF SEHRTQVSLK |
| 481 | DQITSGKYNF IPEVWAEVSE KALDLVKKLL VVDPKARFTT EEALRHPWLQ DEDMKRKFQD |
| 541 | LLSEENESTA LPQVLAQPST SRKRPREGEA EGAETTKRPA VCAAVL. |

In some embodiments of the compositions and methods of the disclosure, a CHEK2 protein of the disclosure comprises or consists of the amino acid sequence of (UniProt Accession No. 096017-10 and SEQ ID NO: 61):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL EFRSYSFYLP. |

In some embodiments of the compositions and methods of the disclosure, a CHEK2 protein of the disclosure comprises or consists of the amino acid sequence of (UniProt Accession No. 096017-11 and SEQ ID NO: 62):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEVLVS VGTAGYNRAV DCWSLGVILF ICLSGYPPFS EHRTQVSLKD |
| 121 | QITSGKYNFI PEVWAEVSEK ALDLVKKLLV VDPKARFTTE EALRHPWLQD EDMKRKFQDL |
| 181 | LSEENESTAL PQVLAQPSTS RKRPREGEAE GAETTKRPAV CAAVL. |

In some embodiments of the compositions and methods of the disclosure, a CHEK2 protein of the disclosure comprises or consists of the amino acid sequence of (UniProt Accession No. 096017-12 and SEQ ID NO: 63):

| | |
|---|---|
| 1 | MSRESDVEAQ QSHGSSACSQ PHGSVTQSQG SSSQSQGISS SSTSTMPNSS QSSHSSSGTL |
| 61 | SSLETVSTQE LYSIPEDQEP EDQEPEEPTP APWARLWALQ DGFANLECVN DNYWFGRDKS |
| 121 | CEYCFDEPLL KRTDKYRTYS KKHFRIFREV GPKNSYIAYI EDHSGNGTFV NTELVGKGKR |
| 181 | RPLNNNSEIA LSLSRNKVFV FFDLTVDDQS VYPKALRDEY IMSKTLGSGA CGEVKLAFER |
| 241 | KTCKKVAIKI ISKRKFAIGS AREADPALNV ETEIEILKKL NHPCIIKIKN FFDAEDYYIV |
| 301 | LELMEGGELF DKVVGNKRLK EATCKLYFYQ MLLAVQITDF GHSKILGETS LMRTLCGTPT |
| 361 | YLAPEVLVSV GTAGYNRAVD CWSLGVILFI CLSGYPPFSE HRTQVSLKDQ ITSGKYNFIP |
| 421 | EVWAEVSEKA LDLVKKLLVV DPKARFTTEE ALRHPWLQDE DMKRKFQDLL SEENESTALP |
| 481 | QVLAQPSTSR KRPREGEAEG AETTKRPAVC AAVL. |

In some embodiments of the compositions and methods of the disclosure, a CHEK2 protein of the disclosure comprises or consists of the amino acid sequence of (UniProt Accession No. 096017-13 and SEQ ID NO: 64):

| | |
|---|---|
| 1 | MSKTLGSGAC GEVKLAFERK TCKKVAIKII SKRKFAIGSA READPALNVE TEIEILKKLN |
| 61 | HPCIIKIKNF FDAEDYYIVL ELMEGGELFD KVVGNKRLKE ATCKLYFYQM LLAVQYLHEN |
| 121 | GIIHRDLKPE NVLLSSQEED CLIKITDFGH SKILGETSLM RTLCGTPTYL APEVLVSVGT |
| 181 | AGYNRAVDCW SLGVILFICL SGYPPFSEHR TQVSLKDQIT SGKYNFIPEV WAEVSEKALD |
| 241 | LVKKLLVVDP KARFTTEEAL RHPWLQDEDM KRKFQDLLSE ENESTALPQV LAQPSTSRKR |
| 301 | PREGEAEGAE TTKRPAVCAA VL. |

Target cells of the disclosure may comprise a modification, such as a gene deletion or mutation, that results in no expression or reduced expression of NBN. Target cells of the disclosure may comprise a modification, such as an insertion, deletion, or substitution in the gene encoding NBN or the encoded NBN protein. Target cells of the disclosure may express a NBN protein according to SEQ ID NO 65. Target cells of the disclosure may express a variant NBN having at least 50%, 55%, 60%, 65%, 70%, 75%, 80%, 85%, 90%, 95%, 97%, 99% or any percentage in between of identity to SEQ ID NO: 65.

In some embodiments of the compositions and methods of the disclosure, a NBN protein of the disclosure comprises or consists of the amino acid sequence of (UniProt Accession No. 060934-1 and SEQ ID NO: 65):

| | |
|---|---|
| 1 | MWKLLPAAGP AGGEPYRLLT GVEYVVGRKN CAILIENDQS ISRNHAVLTA NFSVTNLSQT |
| 61 | DEIPVLTLKD NSKYGTFVNE EKMQNGFSRT LKSGDGITFG VFGSKFRIEY EPLVACSSCL |
| 121 | DVSGKTALNQ AILQLGGFTV NNWTEECTHL VMVSVKVTIK TICALICGRP IVKPEYFTEF |
| 181 | LKAVESKKQP PQIESFYPPL DEPSIGSKNV DLSGRQERKQ IFKGKTFIFL NAKQHKKLSS |
| 241 | AVVFGGGEAR LITEENEEEH NFFLAPGTCV VDTGITNSQT LIPDCQKKWI QSIMDMLQRQ |
| 301 | GLRPIPEAEI GLAVIFMTTK NYCDPQGHPS TGLKTTTPGP SLSQGVSVDE KLMPSAPVNT |
| 361 | TTYVADTESE QADTWDLSER PKEIKVSKME QKFRMLSQDA PTVKESCKTS SNNNSMVSNT |
| 421 | LAKMRIPNYQ LSPTKLPSIN KSKDRASQQQ QTNSIRNYFQ PSTKKRERDE ENQEMSSCKS |
| 481 | ARIETSCSLL EQTQPATPSL WKNKEQHLSE NEPVDTNSDN NLFTDTDLKS IVKNSASKSH |
| 541 | AAEKLRSNKK REMDDVAIED EVLEQLFKDT KPELEIDVKV QKQEEDVNVR KRPRMDIETN |
| 601 | DTFSDEAVPE SSKISQENEI GKKRELKEDS LWSAKEISNN DKLQDDSEML PKKLLLTEFR |
| 661 | SLVIKNSTSR NPSGINDDYG QLKNFKKFKK VTYPGAGKLP HIIGGSDLIA HHARKNTELE |
| 721 | EWLRQEMEVQ NQHAKEESLA DDLFRYNPYL KRRR. |

Target cells of the disclosure may comprise a modification, such as a gene deletion or mutation, that results in no expression or reduced expression of PALB2. Target cells of the disclosure may comprise a modification, such as an insertion, deletion, or substitution in the gene encoding PALB2 or the encoded PALB2 protein. Target cells of the disclosure may express a PALB2 protein according to SEQ ID NO 66. Target cells of the disclosure may express a variant PALB2 having at least 50%, 55%, 60%, 65%, 70%, 75%, 80%, 85%, 90%, 95%, 97%, 99% or any percentage in between of identity to SEQ ID NO: 66.

In some embodiments of the compositions and methods of the disclosure, a PALB2 protein of the disclosure comprises or consists of the amino acid sequence of (UniProt Accession No. Q86YC2-1 and SEQ ID NO: 66):

| | |
|---|---|
| 1 | MDEPPGKPLS CEEKEKLKEK LAFLKREYSK TLARLQRAQR AEKIKHSIKK TVEEQDCLSQ |
| 61 | QDLSPQLKHS EPKNKICVYD KLHIKTHLDE ETGEKTSITL DVGPESFNPG DGPGGLPIQR |
| 121 | TDDTQEHFPH RVSDPSGEQK QKLPSRRKKQ QKRTFISQER DCVFGTDSLR LSGKRLKEQE |
| 181 | EISSKNPARS PVTEIRTHLL SLKSELPDSP EPVTEINEDS VLIPPTAQPE KGVDTFLRRP |
| 241 | NFTRATTVPL QTLSDSGSSQ HLEHIPPKGS SELTTHDLKN IRFTSPVSLE AQGKKMTVST |
| 301 | DNLLVNKAIS KSGQLPTSSN LEANISCSLN ELTYNNLPAN ENQNLKEQNQ TEKSLKSPSD |
| 361 | TLDGRNENLQ ESEILSQPKS LSLEATSPLS AEKHSCTVPE GLLFPAEYYV RTTRSMSNCQ |
| 421 | RKVAVEAVIQ SHLDVKKKGF KNKNKDASKN LNLSNEETDQ SEIRMSGTCT GQPSSRTSQK |
| 481 | LLSLTKVSSP AGPTEDNDLS RKAVAQAPGR RYTGKRKSAC TPASDHCEPL LPTSSLSIVN |
| 541 | RSKEEVTSHK YQHEKLFIQV KGKKSRHQKE DSLSWSNSAY LSLDDDAFTA PFHRDGMLSL |
| 601 | KQLLSFLSIT DFQLPDEDFG PLKLEKVKSC SEKPVEPFES KMFGERHLKE GSCIFPEELS |
| 661 | PKRMDTEMED LEEDLIVLPG KSHPKRPNSQ SQHTKTGLSS SILLYTPLNT VAPDDNDRPT |
| 721 | TDMCSPAFPI LGTTPAFGPQ GSYEKASTEV AGRTCCTPQL AHLKDSVCLA SDTKQFDSSG |
| 781 | SPAKPHTTLQ VSGRQGQPTC DCDSVPPGTP PPIESFTFKE NQLCRNTCQE LHKHSVEQTE |
| 841 | TAELPASDSI NPGNLQLVSE LKNPSGSCSV DVSAMFWERA GCKEPCIITA CEDVVSLWKA |
| 901 | LDAWQWEKLY TWHFAEVPVL QIVPVPDVYN LVCVALGNLE IREIRALFCS SDDESEKQVL |
| 961 | LKSGNIKAVL GLTKRRLVSS SGTLSDQQVE VMTFAEDGGG KENQFLMPPE ETILTFAEVQ |
| 1021 | GMQEALLGTT IMNNIVIWNL KTGQLLKKMH IDDSYQASVC HKAYSEMGLL FIVLSHPCAK |
| 1081 | ESESLRSPVF QLIVINPKTT LSVGVMLYCL PPGQAGRFLE GDVKDHCAAA ILTSGTIAIW |
| 1141 | DLLLGQCTAL LPPVSDQHWS FVKWSGTDSH LLAGQKDGNI FVYHYS. |

Target cells of the disclosure may comprise a modification, such as a gene deletion or mutation, that results in no expression or reduced expression of SLX4. Target cells of the disclosure may comprise a modification, such as an insertion, deletion, or substitution in the gene encoding SLX4 or the encoded SLX4 protein. Target cells of the disclosure may express a SLX4 protein according to SEQ ID NO 67 or 68. Target cells of the disclosure may express a variant SLX4 having at least 50%, 55%, 60%, 65%, 70%, 75%, 80%, 85%, 90%, 95%, 97%, 99% or any percentage in between of identity to SEQ ID NO: 67 or 68.

In some embodiments of the compositions and methods of the disclosure, a SLX4 protein of the disclosure comprises or consists of the amino acid sequence of (UniProt Accession No. Q8IY92-1 and SEQ ID NO: 67):

| | |
|---|---|
| 1 | MKLSVNEAQL GFYLGSLSHL SACPGIDPRS SEDQPESLKT GQMMDESDED FKELCASFFQ |
| 61 | RVKKHGIKEV SGERKTQKAA SNGTQIRSKL KRTKQTATKT KTLQGPAEKK PPSGSQAPRT |
| 121 | KKQRVTKWQA SEPAHSVNGE GGVLASAPDP PVLRETAQNT QTGNQQEPSP NLSREKTREN |
| 181 | VPNSDSQPPP SCLTTAVPSP SKPRTAQLVL QRMQQFKRAD PERLRHASEE CSLEAAREEN |
| 241 | VPKDPQEEMM AGNVYGLGPP APESDAAVAL TLQQEFARVG ASAHDDSLEE KGLFFCQICQ |
| 301 | KNLSAMNVTR REQHVNRCLD EAEKTLRPSV PQIPECPICG KPFLTLKSRT SHLKQCAVKM |
| 361 | EVGPQLLLQA VRLQTAQPEG SSSPPMFSFS DHSRGLKRRG PTSKKEPRKR RKVDEAPSED |
| 421 | LLVAMALSRS EMEPGAAVPA LRLESAFSER IRPEAENKSR KKKPPVSPPL LLVQDSETTG |
| 481 | RQIEDRVALL LSEEVELSST PPLPASRILK EGWERAGQCP PPPERKQSFL WEGSALTGAW |
| 541 | AMEDFYTARL VPPLVPQRPA QGLMQEPVPP LVPPEHSELS ERRSPALHGT PTAGCGSRGP |
| 601 | SPSASQREHQ ALQDLVDLAR EGLSASPWPG SGGLAGSEGT AGLDVVPGGL PLTGFVVPSQ |
| 661 | DKHPDRGGRT LLSLGLLVAD FGAMVNNPHL SDVQFQTDSG EVLYAHKFVL YARCPLLIQY |
| 721 | VNNEGFSAVE DGVLTQRVLL GDVSTEAART FLHYLYTADT GLPPGLSSEL SSLAHRFGVS |
| 781 | ELVHLCEQVP IATDSEGKPW EEKEAENCES RAENFQELLR SMWADEEEEA ETLLKSKDHE |
| 841 | EDQENVNEAE MEEIYEFAAT QRKLLQEERA AGAGEDADWL EGGSPVSGQL LAGVQVQKQW |
| 901 | DKVEEMEPLE PGRDEAATTW EKMGQCALPP PQGQHSGARG AEAPEQEAPE EALGHSSCSS |
| 961 | PSRDCQAERK EGSLPHSDDA GDYEQLFSST QGEISEPSQI TSEPEEQSGA VRERGLEVSH |
| 1021 | RLAPWQASPP HPCRFLLGPP QGGSPRGSHH TSGSSLSTPR SRGGTSQVGS PTLLSPAVPS |
| 1081 | KQKRDRSILT LSKEPGHQKG KERRSVLECR NKGVLMFPEK SPSIDLTQSN PDHSSSRSQK |
| 1141 | SSSKLNEEDE VILLLDSDEE LELEQTKMKS ISSDPLEEKK ALEISPRSCE LFSIIDVDAD |
| 1201 | QEPSQSPPRS EAVLQQEDEG ALPENRGSLG RRGAPWLFCD RESSPSEAST TDTSWLVPAT |
| 1261 | PLASRSRDCS SQTQISSLRS GLAVQAVTQH TPRASVGNRE GNEVAQKFSV IRPQTPPPQT |
| 1321 | PSSCLTPVSP GTSDGRRQGH RSPSRPHPGG HPHSSPLAPH PISGDRAHFS RRFLKHSPPG |
| 1381 | PSFLNQTPAG EVVEVGDSDD EQEVASHQAN RSPPLDSDPP IPIDDCCWHM EPLSPIPIDH |
| 1441 | WNLERTGPLS TSSPSRRMNE AADSRDCRSP GLLDTTPIRG SCTTQRKLQE KSSGAGSLGN |
| 1501 | SRPSFLNSAL WDVWDGEEQR PPETPPPAQM PSAGGAQKPE GLETPKGANR KKNLPPKVPI |
| 1561 | TPMPQYSIME TPVLKKELDR FGVRPLPKRQ MVLKLKEIFQ YTHQTLDSDS EDESQSSQPL |
| 1621 | LQAPHCQTLA SQTYKPSRAG VHAQQEATTG PGAHRPKGPA KTKGPRHQRK HHESITPPSR |
| 1681 | SPTKEAPPGL NDDAQIPASQ ESVATSVDGS DSSLSSQSSS SCEFGAAFES AGEEEGEGEV |
| 1741 | SASQAAVQAA DTDEALRCYI RSKPALYQKV LLYQPFELRE LQAELRQNGL RVSSRRLLDF |
| 1801 | LDTHCITFTT AATRREKLQG RRRQPRGKKK VERN. |

In some embodiments of the compositions and methods of the disclosure, a SLX4 protein of the disclosure comprises or consists of the amino acid sequence of (UniProt Accession No. Q8IY92-2 and SEQ ID NO: 68):

| | |
|---|---|
| 1 | MFSFRCLDEA EKTLRPSVPQ IPECPICGKP FLTLKSRTSH |
| | LKQCAVKMEV GPQLLLQAVR |
| 61 | LQTAQPEGSS SPPMFSFSDH SRGLKRRGPT SKKEPRKRRK |
| | VDEAPSEDLL VAMALSRSEM |
| 121 | EPGAAVPALR LESAFSERIR PEAENKSRKK KPPVSPPLLL |
| | VQDSETTGRQ IEDRVALLLS |
| 181 | EEVELSSTPP LPASRILKEG WERAGQCPPP PERKQSFLWE |
| | GSALTGAWAM EDFYTARLVP |
| 241 | PLVPQRPAQG LMQEPVPPLV PPEHSELSER RSPALHGTPT |
| | AGCGSRGPSP SASQREHQAL |
| 301 | QDLVDLAREG LSASPWPGSG GLAGSEGTAG LDVVPGGLPL |
| | TGFVVPSQDK HPDRGGRTLL |
| 361 | SLGLLVADFG AMVNNPHLSD VQFQTDSGEV LYAHKFVLYA |
| | RCPLLIQYVN NEGFSAVEDG |
| 421 | VLTQRVLLGD VSTEAARTFL HYLYTADTGL PPGLSSELSS |
| | LAHRFGVSEL VHLCEQVPIA |
| 481 | TDSEGKPWEE KEAENCESRA ENFQELLRSM WADEEEEAET |
| | LLKSKDHEED QENVNEAEME |
| 541 | EIYEFAATQR KLLQEERAAG AGEDADWLEG GSPVSGQLLA |
| | GVQVQKQWDK VEEMEPLEPG |
| 601 | RDEAATTWEK MGQCALPPPQ GQHSGARGAE APEQEAPEEA |
| | LGHSSCSSPS RDCQAERKEG |
| 661 | SLPHSDDAGD YEQLFSSTQG EISEPSQITS EPEEQSGAVR |
| | ERGLEVSHRL APWQASPPHP |
| 721 | CRFLLGPPQG GSPRGSHHTS GSSLSTPRSR GGTSQVGSPT |
| | LLSPAVPSKQ KRDRSILTLS |
| 781 | KEPGHQKGKE RRSVLECRNK GVLMFPEKSP SIDLTQSNPD |
| | HSSSRSQKSS SKLNEEDEVI |
| 841 | LLLDSDEELE LEQTKMKSIS SDPLEEKKAL EISPRSCELF |
| | SIIDVDADQE PSQSPPRSEA |
| 901 | VLQQEDEGAL PENRGSLGRR GAPWLFCDRE SSPSEASTTD |
| | TSWLVPATPL ASRSRDCSSQ |
| 961 | TQISSLRSGL AVQAVTQHTP RASVGNREGN EVAQKFSVIR |
| | PQTPPPQTPS SCLTPVSPGT |
| 1021 | SDGRRQGHRS PSRPHPGGHP HSSPLAPHPI SGDRAHFSRR |
| | FLKHSPPGPS FLNQTPAGEV |
| 1081 | VEVGDSDDEQ EVASHQANRS PPLDSDPPIP IDDCCWHMEP |
| | LSPIPIDHWN LERTGPLSTS |
| 1141 | SPSRRMNEAA DSRDCRSPGL LDTTPIRGSC TTQRKLQEKS |
| | SGAGSLGNSR PSFLNSALWD |
| 1201 | VWDGEEQRPP ETPPPAQMPS AGGAQKPEGL ETPKGANRKK |
| | NLPPKVPITP MPQYSIMETP |
| 1261 | VLKKELDRFG VRPLPKRQMV LKLKEIFQYT HQTLDSDSED |
| | ESQSSQPLLQ APHCQTLASQ |
| 1321 | TYKPSRAGVH AQQEATTGPG AHRPKGPAKT KGPRHQRKHH |
| | ESITPPSRSP TKEAPPGLND |
| 1381 | DAQIPASQES VATSVDGSDS SLSSQSSSSC EFGAAFESAG |
| | EEEGEGEVSA SQAAVQAADT |
| 1441 | DEALRCYIRS KPALYQKVLL YQPFELRELQ AELRQNGLRV |
| | SSRRLLDFLD THCITFTTAA |
| 1501 | TRREKLQGRR RQPRGKKKVE RN. |

### DNA Ligase 1 (LIG1)

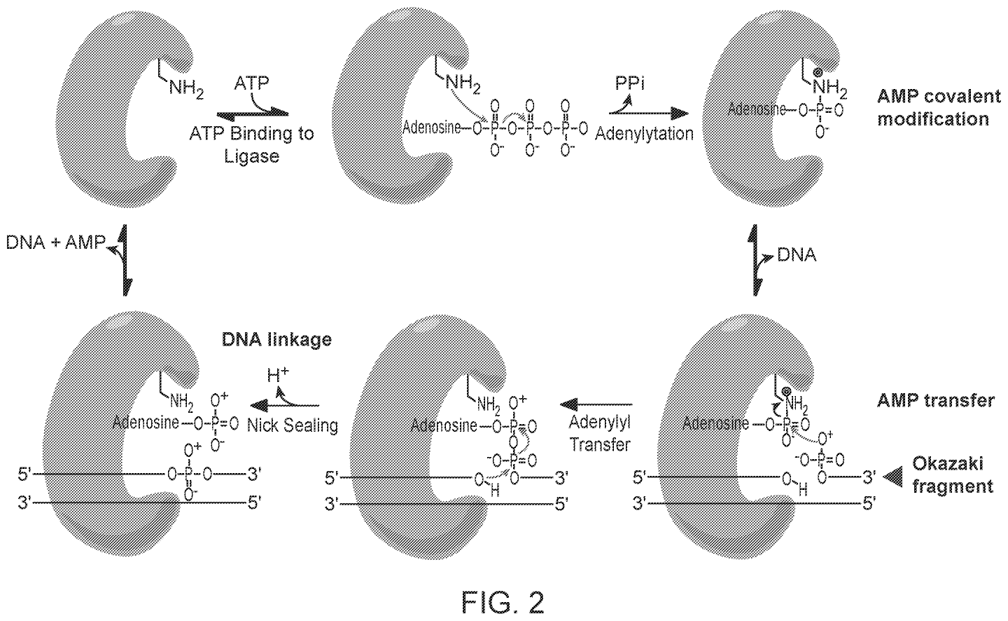

LIG1 is an ATP-dependent DNA ligase. Although there are multiple isoforms (shown below), the ligation mechanism is common to all isoforms (see FIG. 2). LIG1 is involved in DNA replication and repair by joining Okazaki fragments and closing single strand nicks during base excision repair. As used throughout the disclosure, the term Okazaki fragments are meant to describe small fragments of the lagging strand of replication. In some embodiments, Okazaki fragments comprise or consist of between 100 and 200 base pairs, inclusive of the endpoints.

LIG1 is the predominant replicative DNA ligase, but other DNA ligases and/or other LIG1 isoforms may have redundant functions (e.g. LIG3). LIG3 is the only mitochondrial DNA ligase and is therefore essential in mitrochondria. LIG4 is involved in NHEJ and V(D)J recombination.

LIG1 is recruited to sites of replication via interaction with PCNA and RCF.

During the cell cycle, LIG1 has a role in DNA replication. LIG1 is the major ligase responsible for closing nicks in lagging strand. In each human S-phase, 30-50 million Okazaki fragments may be generated and LIG1 is responsible for joining them.

In the process of base excision repair (BER), LIG1 removes single damaged bases. This mechanism of repair is active throughout the cell cycle. During the S-phase, LIG1 is responsible for long-patch BER. Throughout the cell cycle, LIG3 is responsible for short-path BER.

With respect to other repair pathways, LIG1 and LIG3 have overlapping roles in microhomology mediated endjoining (MMEJ) and alternative endjoining (Alt-NHEJ).

In target cells lacking a functional BRCA1 protein or otherwise having an impaired, defective or deregulated HR pathway (even in the absence of variant BRCA1), loss of LIG1 leads to elevation of unligated Okazaki fragments and single strand breaks. HR impairment, deficiency or deregulation prevents detection and repair of unligated fragments. Replication forks collapse during the next cell cycle producing single-ended double strand breaks. A LIG1 blocking agent of the disclosure may increase a number of unligated Okazaki fragments and single strand breaks, which in those cells containing a variant protein that prevents detection and repair of the unligated fragments, induces collapse of one or more replication forks during a phase of the cell cycle producing single-ended double strand breaks. Target cells may have a synergistic interaction or an increased synergy with LIG1 that express a variant protein. In some embodiments, a cell having an impaired, defective or deregulated HR pathway comprises one or more of a variant BRCA1, a variant BRCA2, a variant RAD51, a variant RAD51C, a variant RAD51D, a variant XRCC2, a variant XPF, a variant MRE11A, a variant ATM, a variant BARD1, a variant BRIP1, a variant CHEK1, a variant CHEK2, a variant NBN, a variant PALB2 and a variant SLX4. In some embodiments, a target cell having an impaired, defective or deregulated HR pathway is a Ewing sarcoma cell. In some embodiments, the impaired, defective or deregulated HR pathway is isolated or derived from a Ewing's sarcoma cell.

The human DNA ligase 1 (LIG1) protein is encoded by the LIG1 gene. LIG1 (also known as polydeoxyribonucleotide synthase [ATP] 1) has 3 isoforms. LIG1 may have an additional 7 isoforms from computational analyses (see Uniprot Accession No. P18858).

Blocking agents, compositions or formulations of the disclosure may comprise one or more blocking agents of LIG. Blocking agents may inhibit one or more activity or function of LIG1. In particular embodiments, a LIG1 blocking agent inhibits a LIG1 activity or function such as DNA ligase activity. In particular embodiments, the activity is inhibited by at least 50%, at least 60%, at least 70%, at least 80%, at least 90%, or about 100%.

Blocking agents, compositions or formulations of the disclosure may comprise one or more isoforms of LIG1. Blocking agents, compositions or formulations of the disclosure may comprise a LIG1 variant encoded by an amino acid sequence having at least 50%, 55%, 60%, 65%, 70%, 75%, 80%, 85%, 90%, 95%, 97%, 99% or any percentage in between of identity to a LIG1 protein of the disclosure. Blocking agents, compositions or formulations of the disclosure may comprise a LIG1 variant encoded by an amino acid sequence having at least 50%, 55%, 60%, 65%, 70%, 75%, 80%, 85%, 90%, 95%, 97%, 99% or any percentage in between of identity to one or more of SEQ ID NO 1, 3 and 5.

Blocking agents, compositions or formulations of the disclosure may comprise a LIG1 variant encoded by a nucleic sequence having at least 50%, 55%, 60%, 65%, 70%, 75%, 80%, 85%, 90%, 95%, 97%, 99% or any percentage in between of identity to a nucleic acid sequence encoding a LIG1 protein of the disclosure. Blocking agents, compositions or formulations of the disclosure may comprise a LIG1 variant encoded by a nucleic sequence having at least 50%, 55%, 60%, 65%, 70%, 75%, 80%, 85%, 90%, 95%, 97%, 99% or any percentage in between of identity to SEQ ID NO: 2, 4, and 6.

The amino acid sequence of LIG1, isoform 1, is provided below (see also UniProt Accession No. P18858-1 and GenBank Accession No. NP_000225; SEQ ID NO: 1):

| | |
|---|---|
| 1 | MQRSIMSFFH PKKEGKAKKP EKEASNSSRE TEPPPKAALK |
| | EWNGVVSESD SPVKRPGRKA |
| 61 | ARVLGSEGEE EDEALSPAKG QKPALDCSQV SPPRPATSPE |
| | NNASLSDTSP MDSSPSGIPK |
| 121 | RRTARKQLPK RTIQEVLEEQ SEDEDREAKR KKEEEEEETP |
| | KESLTEAEVA TEKEGEDGDQ |
| 181 | PTTPPKPLKT SKAETPTESV SEPEVATKQE LQEEEEQTKP |
| | PRRAPKTLSS FFTPRKPAVK |
| 241 | KEVKEEEPGA PGKEGAAEGP LDPSGYNPAK NNYHPVEDAC |
| | WKPGQKVPYL AVARTFEKIE |
| 301 | EVSARLRMVE TLSNLLRSVV ALSPPDLLPV LYLSLNHLGP |
| | PQQGLELGVG DGVLLKAVAQ |
| 361 | ATGRQLESVR AEAAEKGDVG LVAENSRSTQ RLMLPPPPLT |
| | ASGVFSKFRD IARLTGSAST |
| 421 | AKKIDIIKGL FVACRHSEAR FIARSLSGRL RLGLAEQSVL |
| | AALSQAVSLT PPGQEFPPAM |
| 481 | VDAGKGKTAE ARKTWLEEQG MILKQTFCEV PDLDRIIPVL |
| | LEHGLERLPE HCKLSPGIPL |
| 541 | KPMLAHPTRG ISEVLKRFEE AAFTCEYKYD GQRAQIHALE |
| | GGEVKIFSRN QEDNTGKYPD |
| 601 | IISRIPKIKL PSVTSFILDT EAVAWDREKK QIQPFQVLTT |
| | RKRKEVDASE IQVQVCLYAF |
| 661 | DLIYLNGESL VREPLSRRRQ LLRENFVETE GEFVFATSLD |
| | TKDIEQIAEF LEQSVKDSCE |
| 721 | GLMVKTLDVD ATYEIAKRSH NWLKLKKDYL DGVGDTLDLV |
| | VIGAYLGRGK RAGRYGGFLL |
| 781 | ASYDEDSEEL QAICKLGTGF SDEELEEHHQ SLKALVLPSP |
| | RPYVRIDGAV IPDHWLDPSA |
| 841 | VWEVKCADLS LSPIYPAARG LVDSDKGISL RFPRFIRVRE |
| | DKQPEQATTS AQVACLYRKQ |
| 901 | SQIQNQQGED SGSDPEDTY. |

The nucleic acid sequence of LIG1, isoform 1, is provided below (see also UniProt Accession No. P18858-1 and GenBank Accession No. NM_000234; SEQ ID NO: 2):

| | |
|---|---|
| 1 | ctcgcggggg cgcttccacc gattcctcct ctttccctgc |
| | cagtcactcc tcagaccctc |
| 61 | agccacaccc gctcatccag ggcgagggaa agcgcgggca |
| | ttttcccagt gtgctctgcg |
| 121 | ggagggctcg ccccacttca ccccttttcc cgccctcctc |
| | ccattcggga gactacgact |
| 181 | cccagtgtcc tccgcgcgac ggcggcggtg cggacggtgc |
| | ccaggtcccg cccctaggct |
| 241 | ctgccccgcc cccgcccgca gacgtctgcg cgcgaatgcc |
| | gtggcgcgaa cttgggactg |
| 301 | cagaggcgcg cctggcggat ctgagtgtgt tgcccgggca |
| | gcggcgcgcg ggaccaacgc |
| 361 | aaggagcagc tgacagacga agaaaagtgc tggacaggaa |
| | gggagaattc tgacgccaac |
| 421 | atgcagcgaa gtatcatgtc atttttccac cccaagaaag |
| | agggtaaagc aaagaagcct |
| 481 | gagaaggagg catccaatag cagcagagag acggagcccc |
| | ctccaaaggc ggcactgaag |
| 541 | gagtggaatg gagtggtgtc cgagagtgac tctccggtga |
| | agaggccagg gaggaaggcg |
| 601 | gcccgggtcc tgggcagcga aggggaagag gaggatgaag |
| | cccttagccc tgctaaaggc |
| 661 | cagaagcctg ccctggactg ctcacaggtc tccccgcccc |
| | gtcctgccac atctcctgag |
| 721 | aacaatgctt ccctctctga cacctctccc atggacagtt |
| | ccccatcagg gattccgaag |
| 781 | cgtcgcacag ctcggaagca gctcccgaaa cggaccattc |
| | aggaagtcct ggaagagcag |
| 841 | agtgaggacg aggacagaga agccaagagg aagaaggagg |
| | aggaagaaga ggagaccccg |
| 901 | aaagaaagcc tcacagaggc tgaagtggcc acagagaagg |
| | aaggagaaga cggggaccag |
| 961 | cccaccacgc ctcccaagcc cctaaagacc tccaaagcag |
| | agaccccgac ggaaagcgtt |
| 1021 | tcagagcctg aggtggccac gaagcaggaa ctgcaggagg |
| | aggaagagca gaccaagcct |
| 1081 | ccccgcagag ctcccaagac gctcagcagc ttcttcaccc |
| | cccggaagcc agcagtcaaa |
| 1141 | aaagaagtga aggaagagga gccaggggct ccaggaaagg |
| | agggagctgc tgagggaccc |
| 1201 | ctggatccat ctggttacaa tcctgccaag aacaactatc |
| | atcccgtgga agatgcctgc |
| 1261 | tggaaaccgg gccagaaggt tccttacctg gctgtggccc |
| | ggacgtttga gaagatcgag |
| 1321 | gaggtgtctg ctcggctccg gatggtggag acgctgagca |
| | acttgctgcg ctccgtggtg |
| 1381 | gccctgtcgc ctccagacct cctccctgtc ctctacctca |
| | gcctcaacca ccttgggcca |
| 1441 | ccccagcagg gcctggagct tggcgtgggt gatggtgtcc |
| | ttctcaaggc agtggcccag |
| 1501 | gccacaggtc ggcagctgga gtccgtccgg gctgaggcag |
| | ccgagaaagg cgacgtgggg |
| 1561 | ctggtggccg agaacagccg cagcacccag aggctcatgc |
| | tgccaccacc tccgctcact |
| 1621 | gcctccgggg tcttcagcaa gttccgcgac atcgccaggc |
| | tcactggcag tgcttccaca |
| 1681 | gccaagaaga tagacatcat caaaggcctc tttgtggcct |
| | gccgccactc agaagcccgg |
| 1741 | ttcatcgcta ggtccctgag cggacggctg cgccttgggc |
| | tggcagagca gtcggtgctg |
| 1801 | gctgccctct cccaggcagt gagcctcacg cccccgggcc |
| | aagaattccc accagccatg |
| 1861 | gtggatgctg ggaagggcaa gacagcagag gccagaaaga |
| | cgtggctgga ggagcaaggc |
| 1921 | atgatcctga agcagacgtt ctgcgaggtt cccgacctgg |
| | accgaattat ccccgtgctg |
| 1981 | ctggagcacg gcctggaacg tctcccggag cactgcaagc |
| | tgagcccagg gattcccctg |
| 2041 | aaaccaatgt tggcccatcc cacccggggc atcagcgagg |
| | tcctgaaacg ctttgaggag |
| 2101 | gcagctttca cctgcgaata caaatatgac gggcagaggg |
| | cacagatcca cgccctggaa |
| 2161 | ggcggggagg tgaagatctt cagcaggaat caggaagaca |
| | acactgggaa gtacccggac |
| 2221 | atcatcagcc gcatccccaa gattaaactc ccatcggtca |
| | catccttcat cctggacacc |
| 2281 | gaagccgtgg cttgggaccg ggaaaagaag cagatccagc |
| | cattccaagt gctcaccacc |
| 2341 | cgcaaacgca aggaggtgga tgcgtctgag atccaggtgc |
| | aggtgtgttt gtacgccttc |
| 2401 | gacctcatct acctcaatgg agagtccctg gtacgtgagc |
| | ccctttcccg gcgccggcag |
| 2461 | ctgctccggg agaactttgt ggagacagag ggcgagtttg |
| | tcttcgccac ctccctggac |
| 2521 | accaaggaca tcgagcagat cgccgagttc ctggagcagt |
| | cagtgaaaga ctcctgcgag |
| 2581 | gggctgatgg tgaagaccct ggatgttgat gccacctacg |
| | agatcgccaa gagatcgcac |
| 2641 | aactggctca agctgaagaa ggactacctt gatggcgtgg |
| | gtgacaccct ggacctggtg |
| 2701 | gtgatcggcg cctacctggg ccgggggaag cgggccggcc |
| | ggtacggggg cttcctgctg |
| 2761 | gcctcctacg acgaggacag tgaggagctg caggccatat |
| | gcaagcttgg aactggcttc |
| 2821 | agtgatgagg agctggagga gcatcaccag agcctcaagg |
| | cgctggtgct gcccagccca |
| 2881 | cgcccttacg tgcggataga tggcgctgtg attcccgacc |
| | actggctgga ccccagcgct |
| 2941 | gtgtgggagg tgaagtgcgc tgacctctcc ctctctccca |
| | tctaccctgc tgcgcggggc |
| 3001 | ctggtggata gtgacaaggg catctccctt cgcttccctc |
| | ggtttattcg agtccgtgaa |
| 3061 | gacaagcagc cggagcaggc caccaccagt gctcaggtgg |
| | cctgtttgta ccggaagcaa |
| 3121 | agtcagattc agaaccaaca aggcgaggac tcaggctctg |
| | accctgaaga tacctactaa |
| 3181 | gccctcgccc tcctagggcc tgggtacagg gcatgagttg |
| | gacggacccc agggttatta |
| 3241 | ttgcctttgc tttttagcaa atctgctgtg gcaggctgtg |
| | gattttgaga gtcaggggag |
| 3301 | gggtgtgtgt gtgagggggt ggcttactcc ggagtctggg |
| | attcatcccg tcatttcttt |
| 3361 | caataaataa ttattggata gctaaaaaaa aaaaaaaaaa |
| | aaaaaaaaaa. |

The amino acid sequence of LIG1, isoform 2, is provided below (see also UniProt Accession No. P18858-2 and GenBank Accession No. AAI10623; SEQ ID NO: 3):

| | |
|---|---|
| 1 | MQRSIMSFFH PKKEGKAKKP EKEASNSSRE TEPPPKAALK |
| | EWNGVVSESD SPVKRPGRKA |
| 61 | ARVLGSEGEE EDEALSPAKG QKPALDCSQV SPPRPATSPE |
| | NNASLSDTSP MDSSPSGIPK |
| 121 | RRTARKQLPK RTIQEVLEEQ SEDEDREAKR KKEEEEEETP |
| | KESLTEAEVA TEKEGEDGDQ |
| 181 | PTTPPKPLKT SKAETPTESV SEPEVATKQE LQEEEEQTKP |
| | PRRAPKTLSS FFTPRKPAVK |
| 241 | KEVKEEEPGA PGKEGAAEGP LDPSGYNPAK NNYHPVEDAC |
| | WKPGQKVPYL AVARTFEKIE |
| 301 | EVSARLRMVE TLSNLLRSVV ALSPPDLLPV LYLSLNHLGP |
| | PQQGLELGVG DGVLLKAVAQ |
| 361 | ATGRQLESVR AEAAEKGDVG LVAENSRSTQ RLMLPPPPLT |
| | ASGVFSKFRD IARLTGSAST |
| 421 | AKKIDIIKGL FVACRHSEAR FIARSLSGRL RLGLAEQSVL |
| | AALSQAVSLT PPGQEFPPAM |
| 481 | VDAGKGKTAE ARKTWLEEQG MILKQTFCEV PDLDRIIPVL |
| | LEHGLERLPE HCKLSPGIPL |
| 541 | KPMLAHPTRG ISEVLKRFEE AAFTCEYKYD GQRAQIHALE |
| | GGEVKIFSRN QEDNTGKYPD |
| 601 | IISRIPKIKL PSVTSFILDT EAVAWDREKK QIQPFQVLTT |
| | RKRKEVDASE IQVQVCLYAF |
| 661 | DLIYLNGESL VREPLSRRRQ LLRENFVETE GEFVFATSLD |
| | TKDIEQIAEF LEQSVKDSCE |
| 721 | GLMVKTLDVD ATYEIAKRSH NWLKLKKDYL DGVGDTLDLV |
| | VIGAYLGRGK RAGRYGGFLL |
| 781 | ASYDEDSEEL QAICKVLGNW G. |

The nucleic acid sequence of LIG1, isoform 2, is provided below (see also UniProt Accession No. P18858-2 and GenBank Accession No. BC110622; SEQ ID NO: 4):

| | |
|---|---|
| 1 | gaccaacgca aggagcagct gacagacgaa gaaaagtgct |
| | ggacaggaag ggagaattct |
| 61 | gatgccaaca tgcagcgaag tatcatgtca tttttccacc |
| | ccaagaaaga gggtaaagca |
| 121 | aagaagcctg agaaggaggc atccaatagc agcagagaga |
| | cggagccccc tccaaaggcg |
| 181 | gcactgaagg agtggaatgg agtggtgtcc gagagtgact |
| | ctccggtgaa gaggccaggg |
| 241 | aggaaggcgg cccgggtcct gggcagcgaa ggggaagagg |
| | aggatgaagc ccttagccct |
| 301 | gctaaaggcc agaagcctgc cctggactgc tcacaggtct |
| | ccccgccccg tcctgccaca |
| 361 | tctcctgaga acaatgcttc cctctctgac acctctccca |
| | tggacagttc cccatcaggg |
| 421 | attccgaagc gtcgcacagc tcggaagcag ctcccgaaac |
| | ggaccattca ggaagtcctg |
| 481 | gaagagcaga gtgaggacga ggacagagaa gccaagagga |
| | agaaggagga ggaagaagag |
| 541 | gagaccccga aagaaagcct cacagaggct gaagtggcaa |
| | cagagaagga aggagaagac |
| 601 | ggggaccagc ccaccacgcc tcccaagccc ctaaagacct |
| | ccaaagcaga gaccccgacg |
| 661 | gaaagcgttt cagagcctga ggtggccacg aagcaggaac |
| | tgcaggagga ggaagagcag |
| 721 | accaagcctc cccgcagagc tcccaagacg ctcagcagct |
| | tcttcacccc ccggaagcca |
| 781 | gcagtcaaaa aagaagtgaa ggaagaggag ccaggggctc |
| | caggaaagga gggagctgct |
| 841 | gagggacccc tcgatccatc tggttacaat cctgccaaga |
| | acaactatca tcccgtggaa |
| 901 | gatgcctgct ggaaaccggg ccagaaggtt ccttacctgg |
| | ctgtggcccg gacgtttgag |
| 961 | aagatcgagg aggtgtctgc tcggctccgg atggtggaga |
| | cgctgagcaa cttgctgcgc |
| 1021 | tccgtggtgg ccctgtcgcc tccagacctc ctccctgtcc |
| | tctacctcag cctcaaccac |
| 1081 | cttgggccac cccagcaggg cctggagctt ggcgtgggtg |
| | atggtgtcct tctcaaggca |
| 1141 | gtggcccagg ccacaggtcg gcagctggag tccgtccggg |
| | ctgaggcagc cgagaaaggc |
| 1201 | gacgtggggc tggtggccga gaacagccgc agcacccaga |
| | ggctcatgct gccaccacct |
| 1261 | ccgctcactg cctccggggt cttcagcaag ttccgcgaca |
| | tcgccaggct cactggcagt |
| 1321 | gcttccacag ccaagaagat agacatcatc aaaggcctct |
| | ttgtggcctg ccgccactca |
| 1381 | gaagcccggt tcatcgctag gtccctgagc ggacggctgc |
| | gccttgggct ggcagagcag |
| 1441 | tcggtgctgg ctgccctctc ccaggcagtg agcctcacgc |
| | ccccgggcca agaattccca |
| 1501 | ccagccatgg tggatgctgg gaagggcaag acagcagagg |
| | ccagaaagac gtggctggag |
| 1561 | gagcaaggca tgatcctgaa gcagacgttc tgcgaggttc |
| | ccgacctgga ccgaattatc |
| 1621 | cccgtgctgc tggagcacgg cctggaacgt ctcccggagc |
| | actgcaagct gagcccaggg |
| 1681 | attcccctga aaccaatgtt ggcccatccc acccggggca |
| | tcagcgaggt cctgaaacgc |
| 1741 | tttgaggagg cagctttcac ctgcgaatac aaatatgacg |
| | ggcagagggc acagatccac |
| 1801 | gccctggaag gcggggaggt gaagatcttc agcaggaatc |
| | aggaagacaa cactgggaag |
| 1861 | tacccggaca tcatcagccg catccccaag attaaactcc |
| | catcggtcac atccttcatc |
| 1921 | ctggacaccg aagccgtggc ttgggaccgg gaaaagaagc |
| | agatccagcc attccaagtg |
| 1981 | ctcaccaccc gcaaacgcaa ggaggtggat gcgtctgaga |
| | tccaggtgca ggtgtgtttg |
| 2041 | tacgccttcg acctcatcta cctcaatgga gagtccctgg |
| | tacgtgagcc cctttcccgg |
| 2101 | cgccggcagc tgctccggga gaactttgtg gagacagagg |
| | gcgagtttgt cttcgccacc |
| 2161 | tccctggaca ccaaggacat cgagcagatc gccgagttcc |
| | tggagcagtc agtgaaagac |
| 2221 | tcctgcgagg ggctgatggt gaagaccctg gatgttgatg |
| | ccacctacga gatcgccaag |
| 2281 | agatcgcaca actggctcaa gctgaagaag gactaccttg |
| | atggcgtggg tgacaccctg |
| 2341 | gacctggtgg tgatcggcgc ctacctgggc cgggggaagc |
| | gggccggccg gtacgggggc |
| 2401 | ttcctgctgg cctcctacga cgaggacagt gaggagctgc |
| | aggccatatg caaggtcctg |
| 2461 | gggaactggg gctgatggca gaagcaggag ggaaggagat |
| | tagactcccg ggggtatttc |
| 2521 | ctattgcaca acccagaccg aggggtgctc acatccccct |
| | gtgaaggatc cgttctcccc |
| 2581 | agtttttatt tactttttct ggttctatga gcaactcttg |
| | cttcatatag aaaaacaggc |
| 2641 | tcggatagaa agatgtcagg aatgtgtgtc agtgagagat |
| | tgagattagc tacaaatagt |
| 2701 | ggtggctcac ataagaaaag aatttttttg ttatgaaatg |
| | agaagtgtgg agggggcagt |
| 2761 | ctgatgccat tagggattca ggttcctcct ctccccacat |
| | ttttttttgt ccccacattt |
| 2821 | tttttttctg ccatgtgcct ctggcctcat catcacaatg |
| | tggctgcagg agttccagcc |
| 2881 | ctcatggcca tgcctgctcc acgcaccagg gaagagcaaa |
| | gggggttgga ccctgagtca |
| 2941 | gcctcagcca gtgaacacct tctgctcaag ttctgttgct |
| | catcactttg ctccatggct |
| 3001 | cacccctagc tgcaaggaat tctgggaaat gtaaagtgat |
| | ttactttttt tcaagttaaa |
| 3061 | gtttttctta aagctgtgca cattttggac tagattttct |
| | tacgtatttc ctatattccc |
| 3121 | ctctccaaca aatttgggtc ttttgccaca gaaacagaac |
| | aaaatatcac ctagaatcgt |
| 3181 | accacccaga gacacgcagt agtgacattt gagggaatgt |
| | tttatttaca tttttgcaaa |
| 3241 | gttgtgatcc ttctctatga gtaattttct tttctgcctt |
| | tttaccctgg aagtactttg |
| 3301 | ttttcagtgt tgtatatgcc cttaagcaat atggcaacag |
| | gacctgaatg cccgcacaag |
| 3361 | agttttcaag tgctgtgccc catagcacat agtgctgtgt |
| | cctgaaccag ctccccgtgg |
| 3421 | ctgtgtgggg aagttgttgc cggctttgtt ctgatggctt |
| | ggaactggct tcagtgacga |
| 3481 | ggagctggag gagcatcacc agagcctcaa ggcgctggtg |
| | ctgcccagcc cacgccctta |
| 3541 | cgtgcggata gatggcgctg tgattcccga ccactggctg |
| | gaccccagcg ctgtgtggga |
| 3601 | ggtgaagtgc gctgacctct ccctctctcc catctaccct |
| | gctgcgcggg gcctggtgga |
| 3661 | tagtgacaag ggcatctccc ttcgcttccc tcggtttatt |
| | cgagtccgtg aagacaagca |
| 3721 | gccggagcag gccaccacca gtgctcaggt ggcctgtttg |
| | taccggaagc aaagtcagat |
| 3781 | tcagaaccaa caaggcgagg actcaggctc tgaccctgaa |
| | gatacctact aagccctcgc |
| 3841 | cctcctaggg cctgggtaca gggcatgagt tggacggacc |
| | ccagggttat tattgccttt |
| 3901 | gctttttagc aaatctgctg tggcaggctg tggattttga |
| | gagtcagggg aggggtg. |

The amino acid sequence of LIG1, isoform 3, is provided below (see also UniProt Accession No. P18858-3 and GenBank Accession No. NP_001275992; SEQ ID NO: 5):

| | |
|---|---|
| 1 | MQRSIMAALK EWNGVVSESD SPVKRPGRKA ARVLGSEGEE |
| | EDEALSPAKG QKPALDCSQV |
| 61 | SPPRPATSPE NNASLSDTSP MDSSPSGIPK RRTARKQLPK |
| | RTIQEVLEEQ SEDEDREAKR |
| 121 | KKEEEEETPK ESLTEAEVAT EKEGEDGDQP TTPPKPLKTS |
| | KAETPTESVS EPEVATKQEL |
| 181 | QEEEEQTKPP RRAPKTLSSF FTPRKPAVKK EVKEEEPGAP |
| | GKEGAAEGPL DPSGYNPAKN |
| 241 | NYHPVEDACW KPGQKVPYLA VARTFEKIEE VSARLRMVET |
| | LSNLLRSVVA LSPPDLLPVL |
| 301 | YLSLNHLGPP QQGLELGVGD GVLLKAVAQA TGRQLESVRA |
| | EAAEKGDVGL VAENSRSTQR |
| 361 | LMLPPPPLTA SGVFSKFRDI ARLTGSASTA KKIDIIKGLF |
| | VACRHSEARF IARSLSGRLR |
| 421 | LGLAEQSVLA ALSQAVSLTP PGQEFPPAMV DAGKGKTAEA |
| | RKTWLEEQGM ILKQTFCEVP |
| 481 | DLDRIIPVLL EHGLERLPEH CKLSPGIPLK PMLAHPTRGI |
| | SEVLKRFEEA AFTCEYKYDG |
| 541 | QRAQIHALEG GEVKIFSRNQ EDNTGKYPDI ISRIPKIKLP |
| | SVTSFILDTE AVAWDREKKQ |
| 601 | IQPFQVLTTR KRKEVDASEI QVQVCLYAFD LIYLNGESLV |
| | REPLSRRRQL LRENFVETEG |
| 661 | EFVFATSLDT KDIEQIAEFL EQSVKDSCEG LMVKTLDVDA |
| | TYEIAKRSHN WLKLKKDYLD |
| 721 | GVGDTLDLVV IGAYLGRGKR AGRYGGFLLA SYDEDSEELQ |
| | AICKLGTGFS DEELEEHHQS |
| 781 | LKALVLPSPR PYVRIDGAVI PDHWLDPSAV WEVKCADLSL |
| | SPIYPAARGL VDSDKGISLR |
| 841 | FPRFIRVRED KQPEQATTSA QVACLYRKQS QIQNQQGEDS |
| | GSDPEDTY. |

The nucleic acid sequence of LIG1, isoform 3, is provided below (see also UniProt Accession No. P18858-3 and GenBank Accession No. NM_001289063; SEQ ID NO: 6):

| | |
|---|---|
| 1 | ctcgcggggg cgcttccacc gattcctcct ctttccctgc |
| | cagtcactcc tcagaccctc |
| 61 | agccacaccc gctcatccag ggcgagggaa agcgcgggca |
| | ttttcccagt gtgctctgcg |
| 121 | ggagggctcg ccccacttca ccccttttcc cgccctcctc |
| | ccattcggga gactacgact |
| 181 | cccagtgtcc tccgcgcgac ggcggcggtg cggacggtgc |
| | ccaggtcccg cccctaggct |
| 241 | ctgccccgcc cccgcccgca gacgtctgcg cgcgaatgcc |
| | gtggcgcgaa cttgggactg |
| 301 | cagaggcgcg cctggcggat ctgagtgtgt tgcccgggca |
| | gcggcgcgcg ggaccaacgc |
| 361 | aaggagcagc tgacagacga agaaaagtgc tggacaggaa |
| | gggagaattc tgacgccaac |
| 421 | atgcagcgaa gtatcatggc ggcactgaag gagtggaatg |
| | gagtggtgtc cgagagtgac |
| 481 | tctccggtga agaggccagg gaggaaggcg gcccgggtcc |
| | tgggcagcga aggggaagag |
| 541 | gaggatgaag cccttagccc tgctaaaggc cagaagcctg |
| | ccctggactg ctcacaggtc |
| 601 | tccccgcccc gtcctgccac atctcctgag aacaatgctt |
| | ccctctctga cacctctccc |
| 661 | atggacagtt ccccatcagg gattccgaag cgtcgcacag |
| | ctcggaagca gctcccgaaa |
| 721 | cggaccattc aggaagtcct ggaagagcag agtgaggacg |
| | aggacagaga agccaagagg |
| 781 | aagaaggagg aggaagagga gaccccgaaa gaaagcctca |
| | cagaggctga agtggccaca |
| 841 | gagaaggaag gagaagacgg ggaccagccc accacgcctc |
| | ccaagcccct aaagacctcc |
| 901 | aaagcagaga ccccgacgga aagcgtttca gagcctgagg |
| | tggccacgaa gcaggaactg |
| 961 | caggaggagg aagagcagac caagcctccc cgcagagctc |
| | ccaagacgct cagcagcttc |
| 1021 | ttcacccccc ggaagccagc agtcaaaaaa gaagtgaagg |
| | aagaggagcc aggggctcca |
| 1081 | ggaaaggagg gagctgctga gggacccctg gatccatctg |
| | gttacaatcc tcccaagaac |
| 1141 | aactatcatc ccgtggaaga tgcctgctgg aaaccgggcc |
| | agaaggttcc ttacctggct |
| 1201 | gtggcccgga cgtttgagaa gatcgaggag gtgtctgctc |
| | ggctccggat ggtggagacg |
| 1261 | ctgagcaact tgctgcgctc cgtggtggcc ctgtcgcctc |
| | cagacctcct ccctgtcctc |
| 1321 | tacctcagcc tcaaccacct tgggccaccc cagcagggcc |
| | tggagcttgg cgtgggtgat |
| 1381 | ggtgtccttc tcaaggcagt ggcccaggcc acaggtcggc |
| | agctggagtc cgtccgggct |
| 1441 | gaggcagccg agaaaggcga cgtggggctg gtggccgaga |
| | acagccgcag cacccagagg |
| 1501 | ctcatgctgc caccacctcc gctcactgcc tccggggtct |
| | tcagcaagtt ccgcgacatc |
| 1561 | gccaggctca ctggcagtgc ttccacagcc aagaagatag |
| | acatcatcaa aggcctcttt |
| 1621 | gtggcctgcc gccactcaga agcccggttc atcgctaggt |
| | ccctgagcgg acggctgcgc |
| 1681 | cttgggctgg cagagcagtc ggtgctggct gccctctccc |
| | aggcagtgag cctcacgccc |
| 1741 | ccgggccaag aattcccacc agccatggtg gatgctggga |
| | agggcaagac agcagaggcc |
| 1801 | agaaagacgt ggctggagga gcaaggcatg atcctgaagc |
| | agacgttctg cgaggttccc |
| 1861 | gacctggacc gaattatccc cgtgctgctg gagcacggcc |
| | tggaacgtct cccggagcac |
| 1921 | tgcaagctga gcccagggat tcccctgaaa ccaatgttgg |
| | cccatcccac ccggggcatc |
| 1981 | agcgaggtcc tgaaacgctt tgaggaggca gctttcacct |
| | gcgaatacaa atatgacggg |
| 2041 | cagagggcac agatccacgc cctggaaggc ggggaggtga |
| | agatcttcag caggaatcag |
| 2101 | gaagacaaca ctgggaagta cccggacatc atcagccgca |
| | tccccaagat taaactccca |
| 2161 | tcggtcacat ccttcatcct ggacaccgaa gccgtggctt |
| | gggaccggga aaagaagcag |
| 2221 | atccagccat tccaagtgct caccacccgc aaacgcaagg |
| | aggtggatgc gtctgagatc |
| 2281 | caggtgcagg tgtgtttgta cgccttcgac ctcatctacc |
| | tcaatggaga gtccctggta |
| 2341 | cgtgagcccc tttcccggcg ccggcagctg ctccgggaga |
| | actttgtgga gacagagggc |
| 2401 | gagtttgtct tcgccacctc cctggacacc aaggacatcg |
| | agcagatcgc cgagttcctg |
| 2461 | gagcagtcag tgaaagactc ctgcgagggg ctgatggtga |
| | agaccctgga tcttgatgcc |
| 2521 | acctacgaga tcgccaagag atcgcacaac tggctcaagc |
| | tgaagaagga ctaccttgat |
| 2581 | ggcgtgggtg acaccctgga cctggtggtg atcggcgcct |
| | acctgggccg ggggaagcgg |
| 2641 | gccggccggt acgggggctt cctgctggcc tcctacgacg |
| | aggacagtga ggagctgcag |
| 2701 | gccatatgca agcttggaac tggcttcagt gatgaggagc |
| | tggaggagca tcaccagagc |
| 2761 | ctcaaggcgc tggtgctgcc cagcccacgc ccttacgtgc |
| | ggatagatgg cgctgtgatt |
| 2821 | cccgaccact ggctggaccc cagcgctgtg tgggaggtga |
| | agtgcgctga cctctccctc |
| 2881 | tctcccatct accctgctgc gcggggcctg gtggatagtg |
| | acaagggcat ctcccttcgc |
| 2941 | ttccctcggt ttattcgagt ccgtgaagac aagcagccgg |
| | agcaggccac caccagtgct |
| 3001 | caggtggcct gtttgtaccg gaagcaaagt cagattcaga |
| | accaacaagg cgaggactca |
| 3061 | ggctctgacc ctgaagatac ctactaagcc ctcgccctcc |
| | tagggcctgg gtacagggca |
| 3121 | tgagttggac ggaccccagg gttattattg cctttgcttt |
| | ttagcaaatc tgctgtggca |
| 3181 | ggctgtggat tttgagagtc aggggagggg tgtgtgtgtg |
| | agggggtggc ttactccgga |
| 3241 | gtctgggatt catcccgtca tttctttcaa taaataatta |
| | ttggatagct aaaaaaaaaa |
| 3301 | aaaaaaaaaa aaaaaaa. |

### Fanconi Anemia Group M Protein (FANCM)

FANCM is a DEAD/DEAH box (DExD/H box) helicase. FANCM mediates DNA repair through the stabilization of DNA replication fork structures. FANCM can also resolve DNA-RNA hybrids known as R-loops.

FANCM is a component of the Fanconia Anemia (FA) complex. The Fanconia Anemia (FA) complex prevents, inhibits, minimizes or decreases replication-induced interstrand DNA crosslinks.

Although part of the FA complex, FANCM loss-of-function (LoF) mutations (also referred to herein as variants) do not cause Fanconi anemia in humans; however, an increased risk or predisposition for the development of cancers such as breast cancer have been observed. Additionally, FANCM promotes telomere maintanence in cells utilizing the alternative lengthening of telomere (ALT) pathway. Cells utilizing the ALT pathway are referred to as ALT cells or ALT+ cells, whereas cells not utilizing the ALT pathway are referred to as not being ALT cells or as being ALT− cells.

The human Fanconi Anemia Group M protein (FANCM) protein is encoded by the FANCM gene (also known as KIAA1596). FANCM (also known as Fanconi anemia group M protein (FACM, ATP-dependent RNA helicase FANCM, Fanconi anemia-associated polypeptide of 250 kDa (FAAP250) and Protein Hef ortholog) has 3 isoforms. FANCM may have an additional 4 isoforms from computational analyses (see Uniprot Accession No. Q8IYD8).

Blocking agents, compositions or formulations of the disclosure may comprise one or more blocking agents of FANCM. Blocking agents may inhibit one or more activity or function of FANCM. In particular embodiments, a FANCM blocking agent inhibits a FANCM activity or function selected from ATP-binding, nucleotide-binding, DNA-binding, DNA remodeling, DNA strand separation, DNA-RNA strand separation, or catalyzing the break of a chemical bond using water. In certain embodiments, a FANCM blocking agent inhibits FANCM helicase activity, hydrolase activity, translocase activity, or ATPase activity. In particular embodiments, the activity is inhibited by at least 50%, at least 60%, at least 70%, at least 80%, at least 90%, or about 100%.

Blocking agents, compositions or formulations of the disclosure may inhibit one or more isoforms of FANCM in a target cell, including any of those having an amino acid sequence set forth in any of SEQ ID NOs: 7, 9, or 11. Blocking agents, compositions or formulations of the disclosure may comprise one or more isoforms of FANCM. Blocking agents, compositions or formulations of the disclosure may comprise a FANCM variant encoded by an amino acid sequence having at least 50%, 55%, 60%, 65%, 70%, 75%, 80%, 85%, 90%, 95%, 97%, 99% or any percentage in between of identity to a FANCM protein of the disclosure. Blocking agents, compositions or formulations of the disclosure may comprise a FANCM variant encoded by an amino acid sequence having at least 50%, 55%, 60%, 65%, 70%, 75%, 80%, 85%, 90%, 95%, 97%, 99% or any percentage in between of identity to one or more of SEQ ID NOs: 7, 9 and 11.

Blocking agents, compositions or formulations of the disclosure may comprise a FANCM variant encoded by a nucleic sequence having at least 50%, 55%, 60%, 65%, 70%, 75%, 80%, 85%, 90%, 95%, 97%, 99% or any percentage in between of identity to a nucleic acid sequence encoding a FANCM protein of the disclosure. Blocking agents, compositions or formulations of the disclosure may comprise a FANCM variant encoded by a nucleic sequence having at least 50%, 55%, 60%, 65%, 70%, 75%, 80%, 85%, 90%, 95%, 97%, 99% or any percentage in between of identity to SEQ ID NO: 8, 10 and 12.

The amino acid sequence of FANCM, isoform 1, is provided below (see also UniProt Accession No. Q8IYD8-1 and GenBank Accession No. NP_065988; SEQ ID NO: 7):

| | |
|---|---|
| 1 | MSGRQRTLFQ TWGSSISRSS GTPGCSSGTE RPQSPGSSKA |
| | PLPAAAEAQL ESDDDVLLVA |
| 61 | AYEAERQLCL ENGGFCTSAG ALWIYPTNCP VRDYQLHISR |
| | AALFCNTLVC LPTGLGKTFI |
| 121 | AAVVMYNFYR WFPSGKVVFM APTKPLVTQQ IEACYQVMGI |
| | PQSHMAEMTG STQASTRKEI |
| 181 | WCSKRVLFLT PQVMVNDLSR GACPAAEIKC LVIDEAHKAL |
| | GNYAYCQVVR ELVKYTNHFR |
| 241 | ILALSATPGS DIKAVQQVIT NLLIGQIELR SEDSPDILTY |
| | SHERKVEKLI VPLGEELAAI |
| 301 | QKTYIQILES FARSLIQRNV LMRRDIPNLT KYQIILARDQ |
| | FRKNPSPNIV GIQQGIIEGE |
| 361 | FAICISLYHG YELLQQMGMR SLYFFLCGIM DGTKGMTRSK |
| | NELGRNEDFM KLYNHLECMF |
| 421 | ARTRSTSANG ISAIQQGDKN KKFVYSHPKL KKLEEVVIEH |
| | FKSWNAENTT EKKRDETRVM |
| 481 | IFSSFRDSVQ EIAEMLSQHQ PIIRVMTFVG HASGKSTKGF |
| | TQKEQLEVVK QFRDGGYNTL |
| 541 | VSTCVGEEGL DIGEVDLIIC FDSQKSPIRL VQRMGRTGRK |
| | RQGRIVIILS EGREERIYNQ |
| 601 | SQSNKRSIYK AISSNRQVLH FYQRSPRMVP DGINPKLHKM |
| | FITHGVYEPE KPSRNLQRKS |
| 661 | SIFSYRDGMR QSSLKKDWFL SEEEFKLWNR LYRLRDSDEI |
| | KEITLPQVQF SSLQNEENKP |
| 721 | AQESTTGIHQ LSLSEWRLWQ DHPLPTHQVD HSDRCRHFIG |
| | LMQMIEGMRH EEGECSYELE |
| 781 | VESYLQMEDV TSTFIAPRNE SNNLASDTFI THKKSSFIKN |
| | INQGSSSSVI ESDEECAEIV |
| 841 | KQTHIKPTKI VSLKKKVSKE IKKDQLKKEN NHGIIDSVDN |
| | DRNSTVENIF QEDLPNDKRT |
| 901 | SDTDEIAATC TINENVIKEP CVLLTECQFT NKSTSSLAGN |
| | VLDSGYNSFN DEKSVSSNLF |
| 961 | LPFEEELYIV RTDDQFYNCH SLTKEVLANV ERFLSYSPPP |
| | LSGLSDLEYE IAKGTALENL |
| 1021 | LFLPCAEHLR SDKCTCLLSH SAVNSQQNLE LNSLKCINYP |
| | SEKSCLYDIP NDNISDEPSL |
| 1081 | CDCDVHKHNQ NENLVPNNRV QIHRSPAQNL VGENNHDVDN |
| | SDLPVLSTDQ DESLLLFEDV |
| 1141 | NTEFDDVSLS PLNSKSESLP VSDKTAISET PLVSQFLISD |
| | ELLLDNNSEL QDQITRDANS |
| 1201 | FKSRDQRGVQ EEKVKNHEDI FDCSRDLFSV TFDLGFCSPD |
| | SDDEILEHTS DSNRPLDDLY |
| 1261 | GRYLEIKEIS DANYVSNQAL IPRDHSKNFT SGTVIIPSNE |
| | DMQNPNYVHL PLSAAKNEEL |
| 1321 | LSPGYSQFSL PVQKKVMSTP LSKSNTLNSF SKIRKEILKT |
| | PDSSKEKVNL QRFKEALNST |
| 1381 | FDYSEFSLEK SKSSGPMYLH KSCHSVEDGQ LLTSNESEDD |
| | EIFRRKVKRA KGNVLNSPED |
| 1441 | QKNSEVDSPL HAVKKRRFPI NRSELSSSDE SENFPKPCSQ |
| | LEDFKVCNGN ARRGIKVPKR |
| 1501 | QSHLKHVARK FLDDEAELSE EDAEYVSSDE NDESENEQDS |
| | SLLDFLNDET QLSQAINDSE |
| 1561 | MRAIYMKSLR SPMMNNKYKM IHKTHKNINI FSQIPEQDET |
| | YLEDSFCVDE EESCKGQSSE |
| 1621 | EEVCVDFNLI TDDCFANSKK YKTRRAVMLK EMMEQNCAHS |
| | KKKLSRIILP DDSSEEENNV |
| 1681 | NDKRESNIAV NPSTVKKNKQ QDHCLNSVPS GSSAQSKVRS |
| | TPRVNPLAKQ SKQTSLNLKD |
| 1741 | TISEVSDFKP QNHNEVQSTT PPFTTVDSQK DCRKFPVPQK |
| | DGSALEDSST SGASCSKSRP |
| 1801 | HLAGTHTSLR LPQEGKGTCI LVGGHEITSG LEVISSLRAI |
| | HGLQVEVCPL NGCDYIVSNR |
| 1861 | MVVERRSQSE MLNSVNKNKF IEQIQHLQSM FERICVIVEK |
| | DREKTGDTSR MFRRTKSYDS |
| 1921 | LLTTLIGAGI RILFSSCQEE TADLLKELSL VEQRKNVGIH |
| | VPTVVNSNKS EALQFYLSIP |
| 1981 | NISYITALNM CHQFSSVKRM ANSSLQEISM YAQVTHQKAE |
| | EIYRYIHYVF DIQMLPNDLN |
| 2041 | QDRLKSDI. |

The nucleic acid sequence of FANCM, isoform 1, is provided below (see also UniProt Accession No. Q8IYD8-1 and GenBank Accession No. NM_020937; SEQ ID NO: 8):

| | |
|---|---|
| 1 | tgtgcgaagg aaaccgatgg ggatcggaac cgtagcggtt gagctgctgc tgctacggat |
| 61 | atctgacaga agccttcggt ggttgtcggc ctaatgagcg gacggcaaag aacgcttttt |
| 121 | cagacgtggg gctcaagtat ctcccgatca tctgggactc cgggttgcag ctccggaact |
| 181 | gagcgacctc agagccctgg cagctccaag gcgcctttgc cagcagcagc ggaggctcag |
| 241 | ctggagtcgg acgatgatgt gttgcttgtc gcggcgtacg aggctgagcg gcagttgtgt |
| 301 | ctagagaatg gcgggttctg cacctccgcg ggcgccctgt ggatttaccc taccaattgc |
| 361 | ccagtgcggg actaccagct gcacatttcc cgggctgctc tgttttgcaa tacgctggtg |
| 421 | tgtctgccta ccggactggg aaagaccttt attgccgccg tggtcatgta caatttctac |
| 481 | cgctggttcc cttcaggaaa ggtggtcttc atggccccaa cgaaaccctt ggtgacacag |
| 541 | cagatcgagg cttgctacca ggtgatgggt atcccgcaat cccacatggc cgaaatgaca |
| 601 | gggtctacac aagcttccac caggaaggaa atatggtgca gtaagagagt gctttttctt |
| 661 | acacctcagg tcatggtaaa tgacctttct agaggagctt gtcccgctgc tgaaataaag |
| 721 | tctttagtta ttgatgaagc tcataaagct ctcggaaact atgcttattg ccaggttgta |
| 781 | agagaactag tcaaatatac aaatcacttt agaatcttgg ctctaagtgc cacaccaggt |
| 841 | agtgatataa aggctgtgca acaagttatt actaacctgc taattgggca gatagagctt |
| 901 | cgttctgaag attctccaga tattttgaca tattctcatg aaagaaaagt tgaaaagctt |
| 961 | attgttccgc ttggtgaaga acttgcagcc atccaaaaga cctatatcca gattttggaa |
| 1021 | tcatttgctc gttctttgat tcagaggaat gttttgatga gaagggatat cccaaatcta |
| 1081 | acaaaatatc agataattct ggcaagagat cagtttagga aaaacccatc tccgaatatt |
| 1141 | gtgggaatac aacaaggcat aatcgaggga gagtttgcta tttgtattag tttatatcat |
| 1201 | ggttatgaat tattgcagca aatgggaatg agatcattat atttcttcct ttgtggaatt |
| 1261 | atggatggaa ctaaagggat gacacggtca aaaaatgaac ttggccgaaa tgaagacttc |
| 1321 | atgaaactct ataatcatct agagtgtatg tttgcacgta cacgtagtac ttcagcaaat |
| 1381 | ggtatttctg ctatccaaca aggagataaa aataaaaaat ttgtttatag tcatccaaag |
| 1441 | ttaaagaaat tagaagaagt tgtaattgaa cacttcaagt catggaatgc tgaaaacact |
| 1501 | actgaaaaga aacgtgatga gacccgagtt atgatcttct cttcatttcg agatagtgtt |
| 1561 | caagaaattg cagaaatgct ttcacagcat cagccaatta ttagagtaat gacttttgtc |
| 1621 | ggccatgcct cagggaaaag cacgaagggt tttacccaga aggagcaact ggaggtagtg |
| 1681 | aaacagtttc gtgacggtgg ttacaacacg ctggtttcta cctgtgtggg tgaagaaggt |
| 1741 | ttggatatag gagaagttga tcttataata tgttttgatt cccagaagag cccaattcgt |
| 1801 | cttgtacaac gaatgggtag aactggccgt aaacgtcaag gcaggatagt tattatcctt |
| 1861 | tctgaaggac gagaggaacg tatttataat cagagtcagt ccaacaaaag aagtatatat |
| 1921 | aaagctattt caagtaacag gcaggtcctt catttttacc aaagaagtcc acgaatggtt |
| 1981 | cctgatggaa tcaacccaaa attacacaaa atgttcatca cacatggtgt ctatgaacca |
| 2041 | gagaagcctt ctcggaactt gcagcgaaag tcatctatct tttcctatag ggatggaatg |
| 2101 | aggcaaagta gcctaaagaa agattggttc ttatcagaag aagaatttaa attatggaac |
| 2161 | agactttata gattaaggga cagtgatgaa attaaagaga taacattgcc tcaagttcag |
| 2221 | ttttcttctt tacaaaatga ggaaaacaaa ccagctcaag aatcaaccac tggaattcat |
| 2281 | caactctctc tctctgaatg gagactgtgg caagatcatc ctttgcctac acatcaagtt |
| 2341 | gatcactcag atcgatgccg ccattttata ggccttatgc aaatgataga gggaatgaga |
| 2401 | cacgaagagg gagaatgcag ctatgaattg gaagttgaat cttatttaca aatggaagat |
| 2461 | gttacctcaa catttattgc tcccaggaat gaatctaata atcttgccag tgacaccttt |
| 2521 | atcactcaca agaaatcgtc atttataaag aacataaatc aaggcagttc atcctcagtg |
| 2581 | atagaatctg atgaagaatg tgctgaaatt gttaaacaaa ctcatatcaa acctactaaa |
| 2641 | attgtttctt taaagaaaaa agtgtctaaa gaaataaaaa aagatcagct taaaaaagaa |
| 2701 | aataatcacg gtattataga ttctgtagat aatgacagaa attccactgt tgaaaatatt |
| 2761 | tttcaagaag acctaccaaa tgataaaagg acatcagata cagatgaaat tgctgccaca |
| 2821 | tgtactatta atgaaaatgt tattaaagaa ccgtgtgtgt tattaacaga gtgtcagttt |
| 2881 | acaaataaat ccactagttc acttgctgga aatgttttag attctggtta taacagtttc |
| 2941 | aatgatgaaa aatctgtttc atctaactta tttcttccat tcgaagaaga gctttatatt |
| 3001 | gttagaacag atgaccaatt ttataattgt cactcattga caaaagaggt actagctaat |
| 3061 | gtagagagat ttttatctta ttctcctccg cctctcagtg gactctcaga cttggaatat |
| 3121 | gaaattgcta agggtactgc acttgagaat ttgcttttct taccctgtgc agagcattta |
| 3181 | cgaagtgata aatgcacctg tttgctgtca cattcagctg tgaattctca acagaattta |
| 3241 | gaattgaatt cacttaaatg tataaattat ccatctgaaa aaagttgcct ttatgatata |
| 3301 | cctaatgata atatttctga tgagccaagt ctctgtgact gtgatgtaca taaacataat |
| 3361 | caaaatgaaa atttagtacc taacaatcgt gttcaaatac acagaagccc tgcacagaat |
| 3421 | ttagttggag agaacaatca tgatgttgat aacagtgacc tcccagtatt gtccactgat |
| 3481 | caagatgaaa gtttgctgtt atttgaagat gttaatacag agttcgacga tgtgagtctt |
| 3541 | tcacccttga acagtaaaag cgaatcttta cctgtgtcag acaaaactgc tattagtgaa |
| 3601 | acgcctctgg tctctcagtt cttaatttct gatgaacttt tgttggacaa taattctgaa |
| 3661 | ctccaagatc aaatcacccg tgatgctaat agttttaaat ctcgtgatca gagaggtgta |
| 3721 | caggaagaaa aagtgaagaa tcatgaggat atttttgatt gctctaggga tttattttct |
| 3781 | gttacctttg atttaggatt ctgtagtcca gattctgatg atgaaatatt ggaacataca |
| 3841 | tcagatagca atagacctct agatgatcta tatggaaggt atttggaaat taaggagata |
| 3901 | agtgatgcaa attatgtttc gaatcaagca ctaataccaa gagatcatag taaaaatttt |
| 3961 | actagtggaa ctgttattat cccatcaaat gaagatatgc agaatccaaa ttatgtacat |
| 4021 | ttgccactga gtgcagcaaa aaatgaagaa ttgttatctc ctggttattc tcagttttct |
| 4081 | ttaccagtgc aaaaaaaagt tatgagtaca ccactctcta aatcaaacac attgaactca |
| 4141 | ttttctaaga taagaaagga aatacttaag acaccagatt ctagtaagga aaaagtaaac |
| 4201 | ctacaaagat tcaaagaagc attgaattca acttttgatt attcagaatt ttctctagaa |
| 4261 | aagtctaaaa gcagtggtcc aatgtatctg cataaatcct gtcattctgt tgaagatgga |
| 4321 | caattattaa caagtaacga aagtgaagat gacgagattt tccgaagaaa agttaaaaga |
| 4381 | gcaaaaggaa atgttttaaa ctctcctgag gatcagaaaa atagtgaagt tgattctcca |
| 4441 | cttcatgctg tcaaaaagcg cagatttcct ataaacagat cagaattatc atctagtgat |
| 4501 | gagagtgaga attttcccaa accatgttca caattagaag acttcaaggt ttgtaacggg |
| 4561 | aatgccagaa gaggcatcaa agtcccaaag agacagagtc acttaaagca tgtagctagg |
| 4621 | aagtttttag atgatgaagc agaactttct gaagaagaty cagaatatgt ttcatcagat |
| 4681 | gaaaatgatg agtcagaaaa tgaacaagat tcctcattac ttgacttttt aaatgatgaa |
| 4741 | actcaacttt cacaggctat aaatgattct gaaatgagag ctatttacat gaaatctttg |
| 4801 | cgtagtccaa tgatgaacaa taagtacaaa atgattcata agacacataa aaacataaac |
| 4861 | attttctcgc agattcctga acaagatgaa acctatttag aggatagttt ttgtgttgat |
| 4921 | gaagaggagt cttgcaaagg ccaatcaagt gaagaagaag tttgtgttga ttttaactta |
| 4981 | ataactgatg attgctttgc aaatagtaaa aagtataaaa ctcgacgtgc agtaatgcta |
| 5041 | aaagaaatga tggaacaaaa ttgtgcacat tcaaaaaaga aattatccag aattatttta |
| 5101 | ccagatgatt caagtgagga ggagaacaat gtaaatgata aaagagaatc taatattgcg |
| 5161 | gttaacccaa gcactgttaa gaagaacaaa caacaggacc attgtttaaa ttcagtgcct |
| 5221 | tctggatctt ctgcgcagtc caaggtgcgt tctactccaa gagttaatcc attagcaaag |
| 5281 | cagagcaaac agacatcgct gaatttaaag gatacaattt ccgaagtctc agacttcaaa |
| 5341 | cctcagaatc ataatgaagt ccagtctacc acaccaccct tcactactgt tgattcacag |
| 5401 | aaagactgta gaaaatttcc agttccacag aaggatggta gtgctttgga ggattctagc |
| 5461 | acttcagggg catcctgttc caagtcaaga ccacatttag ctgggacaca tacttctctt |
| 5521 | agacttccgc aggaaggaaa aggaacctgt attcttgtag gtggtcatga aatcacttct |
| 5581 | ggattagaag taatttcttc cctaagagca attcatgggt tgcaagtaga agtttgtcct |
| 5641 | cttaatggct gtgattacat cgtgagtaat cgcatggtgg tggaaaggag gtctcaatct |
| 5701 | gagatgttaa atagtgtcaa taagaacaag ttcattgagc agatccagca cctgcagagt |
| 5761 | atgtttgaaa gaatatgtgt gattgtggaa aaggacagag aaaaaacagg agacacatca |
| 5821 | aggatgttta ggagaacaaa gagctatgac agcctgctga ctaccttaat tggcgctgga |
| 5881 | atccgaattc ttttcagttc ctgccaagaa gaaaccgcag atttgctaaa ggaactgtct |
| 5941 | ttagtggaac aaagaaagaa tgttggtatt catgttccaa cagtggtgaa tagtaataaa |
| 6001 | agtgaggcac tccagtttta tttaagtatt cccaatataa gttatataac tgcattaaat |
| 6061 | atgtgtcacc agttttcatc tgtgaaaagg atggctaaca gctcacttca agaaatctcc |
| 6121 | atgtatgcac aagtaactca tcagaaggct gaggagatct atagatatat tcactatgta |
| 6181 | tttgacatac aaatgttacc aaatgatctt aaccaagata gactgaaatc tgatatataa |
| 6241 | tcaagctgct caagatgggg ttttcaaaga cctctcacaa tattaaatgc acttcaataa |
| 6301 | tcattgctgt tttatgttta tttgtaaata agagaatatt ttatttaaat attttatatt |
| 6361 | gtatacattt ttatttatag attatagaaa ttattaaaaa agaaaaatct gatgttcagt |
| 6421 | gatcattttg actagattat aaaactaatt tttcttatta aataaaacaa ggtttattaa |
| 6481 | aagtgttact aaggatagtt taagaaagta aaagctaagc tagagatata ctttggaatg |
| 6541 | tttcccaaaa ttaaagttgt actgttgtga taaatagtaa agttgacatg tctatgacta |
| 6601 | cagccaactt gtcgattttc cctatgtgta gatagtatac ttttaagtgt actgattcta |
| 6661 | aatacatgta cttggtaagg tgtgggtgat gggtgggttg tgagataaat gacccagtaa |
| 6721 | ctaggaaagt agaaaactta actgaatgtt tatctgacca aaggtgtgtc ccagttaagt |
| 6781 | actgtcaaat ctattaatat gaactctgat atggtttggc tgtgtcccca accaaaatct |
| 6841 | catcttgact tgtaatctga attataatcc caatatattg gggagggacc tcctggaacg |
| 6901 | tgattagctc atgggggcgg ttcccccatg ctgttctagt gatagttctc agaggatctg |
| 6961 | atggttttat aagcttttcc tctgttcact ctgcagttct cttgcctact gccatgtgga |
| 7021 | aaaggaaacg tttgcttccc ctccaccatg attgtaagtt cccgaggcct ccccagccat |
| 7081 | gcaggactgt gagtcaatta aacatctttt ccttataaat taaaaaaaaa aaaaaaaa |

The amino acid sequence of FANCM, isoform 2, is provided below (see also UniProt Accession No. Q8IYD8-2 and GenBank Accession No. NP_001295063; SEQ ID NO: 9):

| | |
|---|---|
| 1 | MSGRQRTLFQ TWGSSISRSS GTPGCSSGTE RPQSPGSSKA |
| | PLPAAAEAQL ESDDDVLLVA |
| 61 | AYEAERQLCL ENGGFCTSAG ALWIYPTNCP VRDYQLHISR |
| | AALFCNTLVC LPTGLGKTFI |
| 121 | AAVVMYNFYR WFPSGKVVFM APTKPLVTQQ IEACYQVMGI |
| | PQSHMAEMTG STQASTRKEI |
| 181 | WCSKRVLFLT PQVMVNDLSR GACPAAEIKC LVIDEAHKAL |
| | GNYAYCQVVR ELVKYTNHFR |
| 241 | ILALSATPGS DIKAVQQVIT NLLIGQIELR SEDSPDILTY |
| | SHERKVEKLI VPLGEELAAI |
| 301 | QKTYIQILES FARSLIQRNV LMRRDIPNLT KYQIILARDQ |
| | FRKNPSPNIV GIQQGIIEGE |
| 361 | FAICISLYHG YELLQQMGMR SLYFFLCGIM DGTKGMTRSK |
| | NELGRNEDFM KLYNHLECMF |
| 421 | ARTRSTSANG ISAIQQGDKN KKFVYSHPKL KKLEEVVIEH |
| | FKSWNAENTT EKKRDETRVM |
| 481 | IFSSFRDSVQ EIAEMLSQHQ PIIRVMTFVG HASGKSTKGF |
| | TQKEQLEVVK QFRDGGYNTL |
| 541 | VSTCVGEEGL DIGEVDLIIC FDSQKSPIRL VQRMGRTGRK |
| | RQGRIVIILS EGREERIYNQ |
| 601 | SQSNKRSIYK AISSNRQVLH FYQRSPRMVP DGINPKLHKM |
| | FITHGVYEPE KPSRNLQRKS |
| 661 | SIFSYRDGK. |

The nucleic acid sequence of FANCM, isoform 2, is provided below (see also UniProt Accession No. Q8IYD8-2 and GenBank Accession No. NM_001308134; SEQ ID NO: 10):

| | |
|---|---|
| 1 | tgtgcgaagg aaaccgatgg ggatcggaac cgtagcggtt |
| | gagctgctgc tgctacggat |
| 61 | atctgacaga agccttcggt ggttgtcggc ctaatgagcg |
| | gacggcaaag aacgcttttt |
| 121 | cagacgtggg gctcaagtat ctcccgatca tctgggactc |
| | cgggttgcag ctccggaact |
| 181 | gagcgacctc agagccctgg cagctccaag gcgcctttgc |
| | cagcagcagc ggaggctcag |
| 241 | ctggagtcgg acgatgatgt gttgcttgtc gcggcgtacg |
| | aggctgagcg gcagttgtgt |
| 301 | ctagagaatg gcgggttctg cacctccgcg ggcgccctgt |
| | ggatttaccc taccaattgc |
| 361 | ccagtgcggg actaccagct gcacatttcc cgggctgctc |
| | tgttttgcaa tacgctggtg |
| 421 | tgtctgccta ccggactggg aaagaccttt attgccgccg |
| | tggtcatgta caatttctac |
| 481 | cgctggttcc cttcaggaaa ggtggtcttc atggccccaa |
| | cgaaaccctt ggtgacacag |
| 541 | cagatcgagg cttgctacca ggtgatgggt atcccgcaat |
| | cccacatggc cgaaatgaca |
| 601 | gggtctacac aagcttccac caggaaggaa atatggtgca |
| | gtaagagagt gctttttctt |
| 661 | acacctcagg tcatggtaaa tgacctttct agaggagctt |
| | gtcccgctgc tgaaataaag |
| 721 | tctttagtta ttgatgaagc tcataaagct ctcggaaact |
| | atgcttattg ccaggttgta |
| 781 | agagaactag tcaaatatac aaatcacttt agaatcttgg |
| | ctctaagtgc cacaccaggt |
| 841 | agtgatataa aggctgtgca acaagttatt actaacctgc |
| | taattgggca gatagagctt |
| 901 | cgttctgaag attctccaga tattttgaca tattctcatg |
| | aaagaaaagt tgaaaagctt |
| 961 | attgttccgc ttggtgaaga acttgcagcc atccaaaaga |
| | cctatatcca gattttggaa |
| 1021 | tcatttgctc gttctttgat tcagaggaat gttttgatga |
| | gaagggatat cccaaatcta |
| 1081 | acaaaatatc agataattct ggcaagagat cagtttagga |
| | aaaacccatc tccgaatatt |
| 1141 | gtgggaatac aacaaggcat aatcgaggga gagtttgcta |
| | tttgtattag tttatatcat |
| 1201 | ggttatgaat tattgcagca aatgggaatg agatcattat |
| | atttcttcct ttgtggaatt |
| 1261 | atggatggaa ctaaagggat gacacggtca aaaaatgaac |
| | ttggccgaaa tgaagacttc |
| 1321 | atgaaactct ataatcatct agagtgtatg tttgcacgta |
| | cacgtagtac ttcagcaaat |
| 1381 | ggtatttctg ctatccaaca aggagataaa aataaaaaat |
| | ttgtttatag tcatccaaag |
| 1441 | ttaaagaaat tagaagaagt tgtaattgaa cacttcaagt |
| | catggaatgc tgaaaacact |
| 1501 | actgaaaaga aacgtgatga gacccgagtt atgatcttct |
| | cttcatttcg agatagtgtt |
| 1561 | caagaaattg cagaaatgct ttcacagcat cagccaatta |
| | ttagagtaat gacttttgtc |
| 1621 | ggccatgcct cagggaaaag cacgaagggt tttacccaga |
| | aggagcaact ggaggtagtg |
| 1681 | aaacagtttc gtgacggtgg ttacaacacg ctggtttcta |
| | cctgtgtggg tgaagaaggt |
| 1741 | ttggatatag gagaagttga tcttataata tgttttgatt |
| | cccagaagag cccaattcgt |
| 1801 | cttgtacaac gaatgggtag aactggccgt aaacgtcaag |
| | gcaggatagt tattatcctt |
| 1861 | tctgaaggac gagaggaacg tatttataat cagagtcagt |
| | ccaacaaaag aagtatatat |
| 1921 | aaagctattt caagtaacag gcaggtcctt catttttacc |
| | aaagaagtcc acgaatggtt |
| 1981 | cctgatggaa tcaacccaaa attacacaaa atgttcatca |
| | cacatggtgt ctatgaacca |
| 2041 | gagaagcctt ctcggaactt gcagcgaaag tcatctatct |
| | tttcctatag ggatggtaaa |
| 2101 | taaattttgc atttgacaca tgcaaaaaaa aaaaaaaaa. |

The amino acid sequence of FANCM, isoform 3, is provided below (see also UniProt Accession No. Q8IYD8-3 and GenBank Accession No. NP_001295062; SEQ ID NO: 11):

| | |
|---|---|
| 1 | MSGRQRTLFQ TWGSSISRSS GTPGCSSGTE RPQSPGSSKA |
| | PLPAAAEAQL ESDDDVLLVA |
| 61 | AYEAERQLCL ENGGFCTSAG ALWIYPTNCP VRDYQLHISR |
| | AALFCNTLVC LPTGLGKTFI |
| 121 | AAVVMYNFYR WFPSGKVVFM APTKPLVTQQ IEACYQVMGI |
| | PQSHMAEMTG STQASTRKEI |
| 181 | WCSKRVLFLT PQVMVNDLSR GACPAAEIKC LVIDEAHKAL |
| | GNYAYCQAVQ QVITNLLIGQ |
| 241 | IELRSEDSPD ILTYSHERKV EKLIVPLGEE LAAIQKTYIQ |
| | ILESFARSLI QRNVLMRRDI |
| 301 | PNLTKYQIIL ARDQFRKNPS PNIVGIQQGI IEGEFAICIS |
| | LYHGYELLQQ MGMRSLYFFL |
| 361 | CGIMDGTKGM TRSKNELGRN EDFMKLYNHL ECMFARTRST |
| | SANGISAIQQ GDKNKKFVYS |
| 421 | HPKLKKLEEV VIEHFKSWNA ENTTEKKRDE TRVMIFSSFR |
| | DSVQEIAEML SQHQPIIRVM |
| 481 | TFVGHASGKS TKGFTQKEQL EVVKQFRDGG YNTLVSTCVG |
| | EEGLDIGEVD LIICFDSQKS |
| 541 | PIRLVQRMGR TGRKRQGRIV IILSEGREER IYNQSQSNKR |
| | SIYKAISSNR QVLHFYQRSP |
| 601 | RMVPDGINPK LHKMFITHGV YEPEKPSRNL QRKSSIFSYR |
| | DGMRQSSLKK DWFLSEEEFK |
| 661 | LWNRLYRLRD SDEIKEITLP QVQFSSLQNE ENKPAQESTT |
| | GIHQLSLSEW RLWQDHPLPT |
| 721 | HQVDHSDRCR HFIGLMQMIE GMRHEEGECS YELEVESYLQ |
| | MEDVTSTFIA PRNESNNLAS |
| 781 | DTFITHKKSS FIKNINQGSS SSVIESDEEC AEIVKQTHIK |
| | PTKIVSLKKK VSKEIKKDQL |
| 841 | KKENNHGIID SVDNDRNSTV ENIFQEDLPN DKRTSDTDEI |
| | AATCTINENV IKEPCVLLTE |
| 901 | CQFTNKSTSS LAGNVLDSGY NSFNDEKSVS SNLFLPFEEE |
| | LYIVRTDDQF YNCHSLTKEV |
| 961 | LANVERFLSY SPPPLSGLSD LEYEIAKGTA LENLLFLPCA |
| | EHLRSDKCTC LLSHSAVNSQ |
| 1021 | QNLELNSLKC INYPSEKSCL YDIPNDNISD EPSLCDCDVH |
| | KHNQNENLVP NNRVQIHRSP |
| 1081 | AQNLVGENNH DVDNSDLPVL STDQDESLLL FEDVNTEFDD |
| | VSLSPLNSKS ESLPVSDKTA |
| 1141 | ISETPLVSQF LISDELLLDN NSELQDQITR DANSFKSRDQ |
| | RGVQEEKVKN HEDIFDCSRD |
| 1201 | LFSVTFDLGF CSPDSDDEIL EHTSDSNRPL DDLYGRYLEI |
| | KEISDANYVS NQALIPRDHS |
| 1261 | KNFTSGTVII PSNEDMQNPN YVHLPLSAAK NEELLSPGYS |
| | QFSLPVQKKV MSTPLSKSNT |
| 1321 | LNSFSKIRKE ILKTPDSSKE KVNLQRFKEA LNSTFDYSEF |
| | SLEKSKSSGP MYLHKSCHSV |
| 1381 | EDGQLLTSNE SEDDEIFRRK VKRAKGNVLN SPEDQKNSEV |
| | DSPLHAVKKR RFPINRSELS |
| 1441 | SSDESENFPK PCSQLEDFKV CNGNARRGIK VPKRQSHLKH |
| | VARKFLDDEA ELSEEDAEYV |
| 1501 | SSDENDESEN EQDSSLLDFL NDETQLSQAI NDSEMRAIYM |
| | KSLRSPMMNN KYKMIHKTHK |
| 1561 | NINIFSQIPE QDETYLEDSF CVDEEESCKG QSSEEEVCVD |
| | FNLITDDCFA NSKKYKTRRA |
| 1621 | VMLKEMMEQN CAHSKKKLSR IILPDDSSEE ENNVNDKRES |
| | NIAVNPSTVK KNKQQDHCLN |
| 1681 | SVPSGSSAQS KVRSTPRVNP LAKQSKQTSL NLKDTISEVS |
| | DFKPQNHNEV QSTTPPFTTV |
| 1741 | DSQKDCRKFP VPQKDGSALE DSSTSGASCS KSRPHLAGTH |
| | TSLRLPQEGK GTCILVGGHE |
| 1801 | ITSGLEVISS LRAIHGLQVE VCPLNGCDYI VSNRMVVERR |
| | SQSEMLNSVN KNKFIEQIQH |
| 1861 | LQSMFERICV IVEKDREKTG DTSRMFRRTK SYDSLLTTLI |
| | GAGIRILFSS CQEETADLLK |
| 1921 | ELSLVEQRKN VGIHVPTVVN SNKSEALQFY LSIPNISYIT |
| | ALNMCHQFSS VKRMANSSLQ |
| 1981 | EISMYAQVTH QKAEEIYRYI HYVFDIQMLP NDLNQDRLKS |
| | DI. |

The nucleic acid sequence of FANCM, isoform 3, is provided below (see also UniProt Accession No. Q8IYD8-3 and GenBank Accession No. NM_001308133; SEQ ID NO: 12):

| | |
|---|---|
| 1 | ccagagtttt gtgcgaagga aaccgatggg gatcggaacc gtagcggttg agctgctgct |
| 61 | gctacggata tctgacagaa gccttcggtg gttgtcggcc taatgagcgg acggcaaaga |
| 121 | acgctttttc agacgtgggg ctcaagtatc tcccgatcat ctgggactcc gggttgcagc |
| 181 | tccggaactg agcgacctca gagccctggc agctccaagg cgcctttgcc agcagcagcg |
| 241 | gaggctcagc tggagtcgga cgatgatgtg ttgcttgtcg cggcgtacga ggctgagcgg |
| 301 | cagttgtgtc tagagaatgg cgggttctgc acctccgcgg gcgccctgtg gatttaccct |
| 361 | accaattgcc cagtgcggga ctaccagctg cacatttccc gggctgctct gttttgcaat |
| 421 | acgctggtgt gtctgcctac cggactggga aagaccttta ttgccgccgt ggtcatgtac |
| 481 | aatttctacc gctggttccc ttcaggaaag gtggtcttca tggccccaac gaaacccttg |
| 541 | gtgacacagc agatcgaggc ttgctaccag gtgatgggta tcccgcaatc ccacatggcc |
| 601 | gaaatgacag ggtctacaca agcttccacc aggaaggaaa tatggtgcag taagagagtg |
| 661 | ctttttctta cacctcaggt catggtaaat gacctttcta gaggagcttg tcccgctgct |
| 721 | gaaataaagt gtttagttat tgatgaagct cataaagctc tcggaaacta tgcttattgc |
| 781 | caggctgtgc aacaagttat tactaacctg ctaattgggc agatagagct tcgttctgaa |
| 841 | gattctccag atattttgac atattctcat gaaagaaaag ttgaaaagct tattgttccg |
| 901 | cttggtgaag aacttgcagc catccaaaag acctatatcc agattttgga atcatttgct |
| 961 | cgttctttga ttcagaggaa tgttttgatg agaagggata tcccaaatct aacaaaatat |
| 1021 | cagataattc tggcaagaga tcagtttagg aaaaacccat ctccgaatat tgtgggaata |
| 1081 | caacaaggca taatcgaggg agagtttgct atttgtatta gtttatatca tggttatgaa |
| 1141 | ttattgcagc aaatgggaat gagatcatta tatttcttcc tttgtggaat tatggatgga |
| 1201 | actaaaggga tgacacggtc aaaaaatgaa cttggccgaa atgaagactt catgaaactc |
| 1261 | tataatcatc tagagtgtat gtttgcacgt acacgtagta cttcagcaaa tggtatttct |
| 1321 | gctatccaac aaggagataa aaataaaaaa tttgtttata gtcatccaaa gttaaagaaa |
| 1381 | ttagaagaag ttgtaattga acacttcaag tcatggaatg ctgaaaacac tactgaaaag |
| 1441 | aaacgtgatg agacccgagt tatgatcttc tcttcatttc gagatagtgt tcaagaaatt |
| 1501 | gcagaaatgc tttcacagca tcagccaatt attagagtaa tgacttttgt cggccatgcc |
| 1561 | tcagggaaaa gcacgaaggg ttttacccag aaggagcaac tggaggtagt gaaacagttt |
| 1621 | cgtgacggtg gttacaacac gctggtttct acctgtgtgg gtgaagaagg tttggatata |
| 1681 | ggagaagttg atcttataat atgttttgat tcccagaaga gcccaattcg tcttgtacaa |
| 1741 | cgaatgggta gaactggccg taaacgtcaa ggcaggatag ttattatcct ttctgaagga |
| 1801 | cgagaggaac gtatttataa tcagagtcag tccaacaaaa gaagtatata taaagctatt |
| 1861 | tcaagtaaca ggcaggtcct tcatttttac caaagaagtc cacgaatggt tcctgatgga |
| 1921 | atcaacccaa aattacacaa aatgttcatc acacatggtg tctatgaacc agagaagcct |
| 1981 | tctcggaact tgcagcgaaa gtcatctatc ttttcctata gggatggaat gaggcaaagt |
| 2041 | agcctaaaga aagattggtt cttatcagaa gaagaattta aattatggaa cagactttat |
| 2101 | agattaaggg acagtgatga aattaaagag ataacattgc ctcaagttca gttttcttct |
| 2161 | ttacaaaatg aggaaaacaa accagctcaa gaatcaacca ctggaattca tcaactctct |
| 2221 | ctctctgaat ggagactgtg gcaagatcat cctttgccta cacatcaagt tgatcactca |
| 2281 | gatcgatgcc gccattttat aggccttatg caaatgatag agggaatgag acacgaagag |
| 2341 | ggagaatgca gctatgaatt ggaagttgaa tcttatttac aaatggaaga tgttacctca |
| 2401 | acatttattg ctcccaggaa tgaatctaat aatcttgcca gtgacacctt tatcactcac |
| 2461 | aagaaatcgt catttataaa gaacataaat caaggcagtt catcctcagt gatagaatct |
| 2521 | gatgaagaat gtgctgaaat tgttaaacaa actcatatca aacctactaa aattgtttct |
| 2581 | ttaaagaaaa aagtgtctaa agaaataaaa aaagatcagc ttaaaaaaga aaataatcac |
| 2641 | ggtattatag attctgtaga taatgacaga aattccactg ttgaaaatat ttttcaagaa |
| 2701 | gacctaccaa atgataaaag gacatcagat acagatgaaa ttgctgccac atgtactatt |
| 2761 | aatgaaaatg ttattaaaga accgtgtgtg ttattaacag agtgtcagtt tacaaataaa |
| 2821 | tccactagtt cacttgctgg aaatgtttta gattctggtt ataacagttt caatgatgaa |
| 2881 | aaatctgttt catctaactt atttcttcca ttcgaagaag agctttatat tgttagaaca |
| 2941 | gatgaccaat tttataattg tcactcattg acaaaagagg tactagctaa tgtagagaga |
| 3001 | tttttatctt attctcctcc gcctctcagt ggactctcag acttggaata tgaaattgct |
| 3061 | aagggtactg cacttgagaa tttgcttttc ttaccctgtg cagagcattt acgaagtgat |
| 3121 | aaatgcacct gtttgctgtc acattcagct gtgaattctc aacagaattt agaattgaat |
| 3181 | tcacttaaat gtataaatta tccatctgaa aaaagttgcc tttatgatat acctaatgat |
| 3241 | aatatttctg atgagccaag tctctgtgac tgtgatgtac ataaacataa tcaaaatgaa |
| 3301 | aatttagtac ctaacaatcg tgttcaaata cacagaagcc ctgcacagaa tttagttgga |
| 3361 | gagaacaatc atgatgttga taacagtgac ctcccagtat tgtccactga tcaagatgaa |
| 3421 | agtttgctgt tatttgaaga tgttaataca gagttcgacg atgtgagtct ttcacccttg |
| 3481 | aacagtaaaa gcgaatcttt acctgtgtca gacaaaactg ctattagtga aacgcctctg |
| 3541 | gtctctcagt tcttaatttc tgatgaactt ttgttggaca ataattctga actccaagat |
| 3601 | caaatcaccc gtgatgctaa tagttttaaa tctcgtgatc agagaggtgt acaggaagaa |
| 3661 | aaagtgaaga atcatgagga tatttttgat tgctctaggg atttattttc tgttaccttt |
| 3721 | gatttaggat tctgtagtcc agattctgat gatgaaatat tggaacatac atcagatagc |
| 3781 | aatagacctc tagatgatct atatggaagg tatttggaaa ttaaggagat aagtgatgca |
| 3841 | aattatgttt cgaatcaagc actaatacca agagatcata gtaaaaattt tactagtgga |
| 3901 | actgttatta tcccatcaaa tgaagatatg cagaatccaa attatgtaca tttgccactg |
| 3961 | agtgcagcaa aaaatgaaga attgttatct cctggttatt ctcagttttc tttaccagtg |
| 4021 | caaaaaaaag ttatgagtac accactctct aaatcaaaca cattgaactc attttctaag |
| 4081 | ataagaaagg aaatacttaa gacaccagat tctagtaagg aaaaagtaaa cctacaaaga |
| 4141 | ttcaaagaag cattgaattc aacttttgat tattcagaat tttctctaga aaagtctaaa |
| 4201 | agcagtggtc caatgtatct gcataaatcc tgtcattctg ttgaagatgg acaattatta |
| 4261 | acaagtaacg aaagtgaaga tgacgagatt ttccgaagaa aagttaaaag agcaaaagga |
| 4321 | aatgttttaa actctcctga ggatcagaaa aatagtgaag ttgattctcc acttcatgct |
| 4381 | gtcaaaaagc gcagatttcc tataaacaga tcagaattat catctagtga tgagagtgag |
| 4441 | aattttccca aaccatgttc acaattagaa gacttcaagg tttgtaacgg gaatgccaga |
| 4501 | agaggcatca aagtcccaaa gagacagagt cacttaaagc atgtagctag gaagttttta |
| 4561 | gatgatgaag cagaactttc tgaagaagat gcagaatatg tttcatcaga tgaaaatgat |
| 4621 | gagtcagaaa atgaacaaga ttcctcatta cttgactttt taaatgatga aactcaactt |
| 4681 | tcacaggcta taaatgattc tgaaatgaga gctatttaca tgaaatcttt gcgtagtcca |
| 4741 | atgatgaaca ataagtacaa aatgattcat aagacacata aaaacataaa cattttctcg |
| 4801 | cagattcctg aacaagatga aacctattta gaggatagtt tttgtgttga tgaagaggag |
| 4861 | tcttgcaaag gccaatcaag tgaagaagaa gtttgtgttg attttaactt aataactgat |
| 4921 | gattgctttg caaatagtaa aaagtataaa actcgacgtg cagtaatgct aaaagaaatg |
| 4981 | atggaacaaa attgtgcaca ttcaaaaaag aaattatcca gaattatttt accagatgat |
| 5041 | tcaagtgagg aggagaacaa tgtaaatgat aaaagagaat ctaatattgc ggttaaccca |
| 5101 | agcactgtta agaagaacaa acaacaggac cattgtttaa attcagtgcc ttctggatct |
| 5161 | tctgcgcagt ccaaggtgcg ttctactcca agagttaatc cattagcaaa gcagagcaaa |
| 5221 | cagacatcgc tgaatttaaa ggatacaatt tccgaagtct cagacttcaa acctcagaat |
| 5281 | cataatgaag tccagtctac cacaccaccc ttcactactg ttgattcaca gaaagactgt |
| 5341 | agaaaatttc cagttccaca gaaggatggt agtgctttgg aggattctag cacttcaggg |
| 5401 | gcatcctgtt ccaagtcaag accacattta gctgggacac atacttctct tagacttccg |
| 5461 | caggaaggaa aaggaacctg tattcttgta ggtggtcatg aaatcacttc tcgattagaa |
| 5521 | gtaatttctt ccctaagagc aattcatggg ttgcaagtag aagtttgtcc tcttaatggc |
| 5581 | tgtgattaca tcgtgagtaa tcgcatggtg gtggaaagga ggtctcaatc tgagatgtta |
| 5641 | aatagtgtca ataagaacaa gttcattgag cagatccagc acctgcagag tatgtttgaa |
| 5701 | agaatatgtg tgattgtgga aaaggacaga gaaaaaacag gagacacatc aaggatgttt |
| 5761 | aggagaacaa agagctatga cagcctgctg actaccttaa ttggcgctgg aatccgaatt |
| 5821 | cttttcagtt cctgccaaga agaaaccgca gatttgctaa aggaactgtc tttagtggaa |
| 5881 | caaagaaaga atgttggtat tcatgttcca acagtggtga atagtaataa aagtgaggca |
| 5941 | ctccagtttt atttaagtat tcccaatata agttatataa ctgcattaaa tatgtgtcac |
| 6001 | cagttttcat ctgtgaaaag gatggctaac agctcacttc aagaaatctc catgtatgca |
| 6061 | caagtaactc atcagaaggc tgaggagatc tatagatata ttcactatgt atttgacata |
| 6121 | caaatgttac caaatgatct taaccaagat agactgaaat ctgatatata atcaagctgc |
| 6181 | tcaagatggg gttttcaaag acctctcaca atattaaatg cacttcaata atcattgctg |
| 6241 | ttttatgttt atttgtaaat aagagaatat tttatttaaa tattttatat tgtatacatt |
| 6301 | tttatttata gattatagaa attattaaaa aagaaaaatc tgatgttcag tgatcatttt |
| 6361 | gactagatta taaaactaat ttttcttatt aaataaaaca aggtttatta aaagtgttac |
| 6421 | taaggatagt ttaagaaagt aaaagctaag ctagagatat actttggaat gtttcccaaa |
| 6481 | attaaagttg tactgttgtg ataaatagta aagttgacat gtctatgact acagccaact |
| 6541 | tgtcgatttt ccctatgtgt agatagtata cttttaagtg tactgattct aaatacatgt |
| 6601 | acttggtaag gtgtgggtga tgggtgggtt gtgagataaa tgacccagta actaggaaag |
| 6661 | tagaaaactt aactgaatgt ttatctgacc aaaggtgtgt cccagttaag tactgtcaaa |
| 6721 | tctattaata tgaactctga tatggtttgg ctgtgtcccc aaccaaaatc tcatcttgac |
| 6781 | ttgtaatctg aattataatc ccaatatatt ggggagggac ctcctggaac gtgattagct |
| 6841 | catgggggcg gttcccccat gctgttctag tgatagttct cagaggatct gatggtttta |
| 6901 | taagcttttc ctctgttcac tctgcagttc tcttgcctac tgccatgtgg aaaaggaaac |
| 6961 | gtttgcttcc cctccaccat gattgtaagt tcccgaggcc tccccagcca tgcaggactg |
| 7021 | tgagtcaatt aaacatcttt tccttataaa tta. |

### Alt+ Background

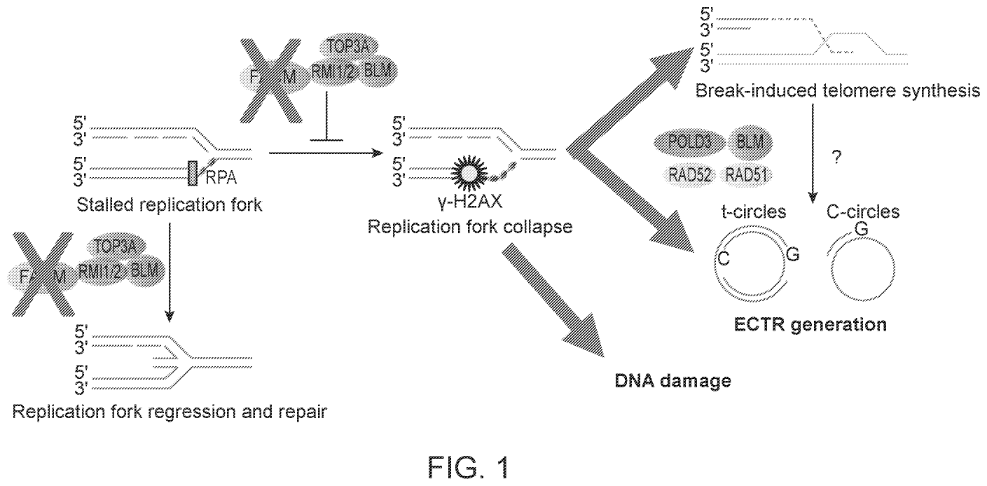

Alternative lengthening of telomeres (ALT) is a telomerase independent mechanism of maintaining telomeres that utilizes break-induced replication stimulated by DSB's thus inducing sustained replicative stress (see FIG. 1). ALT is independent of telomerase and dependent upon homologous recombination. In some embodiments, ALT is mediated directly or indirectly through a function or an activity of one or more of RAD51, RAD51C, RAD51D, XRCC2, XPF, MRE11A, ATM, BARD1, BRIP1, CHEK1, CHEK2, NBN, PALB2 and SLX4. In some embodiments, ALT is mediated directly or indirectly through a function or an activity of one or more of SLX4, RAD51 and MRE11A.

Measurable ALT hallmarks include, but are not limited to, extrachromosomal telomeric DNA (C-circles) that can be measured via rolling circle PCR; absence of telomerase activity; ALT associated telomeric containing promyelocytic leukemia protein (PML) nuclear bodies that can be measured using immunohistochemistry (IHC) assays; Heterogenous telomere sequences that can be measured using whole genome sequencing (WGS) assay; and ATRX/DAXX mutations.

ALT occurs in about 5-10% of cancer. The prevalence is high in mesenchymal tumors and particularly high in sarcomas.

ALT is a marker of poor prognosis in most tumor types (especially soft tissue sarcomas).

The role of ALT in non-cancerous or healthy cells is unknown.

Target cells of the disclosure may demonstrate ALT in addition to or as an alternative to an impaired, defective or deregulated DNA repair pathway. In some embodiments, sarcoma target cells of the disclosure may demonstrate ALT in addition to or as an alternative to an impaired, defective or deregulated DNA repair pathway. In some embodiments, a sarcoma target cell of the disclosure may demonstrate ALT in addition to or as an alternative to an impaired, defective or deregulated DNA repair pathway. In other embodiments, target cells of the disclosure are not ALT+ cells. In some embodiments, breast cancer or ovarian cancer target cells, e.g., comprising a BRCA1 or BRCA2 mutation, are not ALT+ cells, or are not ALT+ cells prior to treatment with the blocking agent.

Composition and methods of the disclosure may increase or enhance a function or an activity of ALT in a target cell. In some embodiments, a FANCM blocking agent of the disclosure may increase or enhance a function or an activity of ALT in a target cell. In some embodiments, the FANCM blocking agent induces increased or enhanced a function of ALT in a target cell. In some embodiments, the increased or enhanced function of ALT induces DNA damage. In some embodiments, the FANCM blocking agent induces uncontrolled ALT and DNA damage in ALT+ cells. In cells having uncontrolled ALT, the threshold at which the accumulation of DNA damage sufficient to cause cell cycle arrest and/or induce programmed cell death may lower than in a cell not having uncontrolled ALT.

FANCM is a component of multiple repair pathways and is required for genome protection against Interstrand crosslinks (ICLs). ICLs obstruct both replication and transcription. FANCM binding at ICLs recruits a core FA complex and ATR-dependent checkpoint activation. The activation of the cell cycle checkpoint stalls the complex at the stalled replication forks referred to as the replisome. ICLs can lead to double strand breaks in a DNA sequence if they are not resolved. In this context, the HR repair pathway involves DSB end resection, strand invasion and Holliday junction resolution. When a cell having an impaired, defective or deregulated HR pathway contacts a FANCM blocking agent, the synergistic combination results in DNA damage sufficient to cause cell cycle arrest and/or induce programmed cell death.

Target cells of the disclosure having an impaired, defective or deregulated HR pathway may increase their dependence upon FANCM and FANCM-mediated repair. This increased dependence upon FANCM may occur in the absence of one or more ICL(s). This increased dependence upon FANCM neither requires nor depends upon the occurrence of one or more ICL(s).

### Methods

Compositions and methods of the disclosure may be used to reduce or inhibit survival or growth of target cells, e.g., cells comprising an impaired, defective or deregulated DNA repair pathway, including any of those described herein. In particular embodiments, the target cells comprise an impaired HR repair pathway. In certain embodiments, the cells are BRCA negative (BRCA^{-/-}) cells. In some embodiments, the BRCA is BRCA1 and/or BRCA2. Thus, in certain embodiments, target cells are BRCA1^{-/-} or BRCA2^{-/-}. In certain embodiments, the cells are tumor cells.

Compositions and methods of the disclosure may be used to induce or increase DNA damage and/or cell cycle arrest of target cells, e.g., cells comprising an impaired, defective or deregulated DNA repair pathway, including any of those described herein. In particular embodiments, the target cells comprise an impaired HR repair pathway. In certain embodiments, the cells are BRCA negative (BRCA^{-/-}) cells. In some embodiments, the BRCA is BRCA1 and/or BRCA2. Thus, in certain embodiments, target cells are BRCA1^{-/-} or BRCA2^{-/-}. In certain embodiments, the cells are tumor cells.

Composition and methods of the disclosure may be used to treat a disease or disorder. In some embodiments, the disease or disorder is a proliferative disorder or a disorder characterized by an aberrant or uncontrolled proliferation of one or more cells or types of cells. In some embodiments, the disease or disorder is a cancer. In some embodiments, the disease or disorder is a sarcoma. In some embodiments, the disease or disorder is a cancer of a bone or connective tissue. In some embodiments, the disease or disorder is a cancer of an epithelial tissue or of an organ or tissue comprising an epithelial cell. In some embodiments, the disease or disorder is an ovarian cancer.

Exemplary proliferative disorders of the disclosure include, but are not limited to, benign proliferative disorders (e.g. benign tumor that causes harm by blockage), oncogenic proliferative disorders (e.g. cancer, metastatic cancer), immunoproliferative disorders (e.g. inflammation and autoimmune disorders), Lymphoproliferative disorders, and myeloproliferative disorders.

Exemplary cancers of the disclosure include, but are not limited to, Acute Lymphocytic Leukemia (ALL) in Adults, Acute Myeloid Leukemia (AML) in Adults, Adrenal Cancer, Anal Cancer, Basal and Squamous Cell Skin Cancer, Bile Duct Cancer, Bladder Cancer, Bone Cancer, Brain and Spinal Cord Tumors in Adults, Brain and Spinal Cord Tumors in Children, Breast Cancer, Breast Cancer in Men, Cancer in Adolescents, Cancer in Children, Cancer in Young Adults, Cancer of Unknown Primary, Castleman Disease, Cervical Cancer, Chronic Lymphocytic Leukemia (CLL), Chronic Myeloid Leukemia (CIVIL), Chronic Myelomonocytic Leukemia (CMML), Colorectal Cancer, Endometrial Cancer, Esophagus Cancer, Ewing Family of Tumors, Ewing Sarcoma, Eye Cancer (Ocular Melanoma), Gallbladder Cancer, Gastrointestinal Neuroendocrine (Carcinoid) Tumors, Gastrointestinal Stromal Tumor (GIST), Gestational Trophoblastic Disease, Hodgkin Lymphoma, Kaposi Sarcoma, Kidney Cancer, Laryngeal and Hypopharyngeal Cancer, Leukemia, Leukemia in Children, Liver Cancer, Lung Cancer, Lung Carcinoid Tumor, Lymphoma, Lymphoma of the Skin, Malignant Mesothelioma, Melanoma Skin Cancer, Merkel Cell Skin Cancer, Multiple Myeloma, Myelodysplastic Syndromes, Nasal Cavity and Paranasal Sinuses Cancer, Nasopharyngeal Cancer, Neuroblastoma, Non-Hodgkin Lymphoma, Oral Cavity and Oropharyngeal Cancer, Osteosarcoma, Ovarian Cancer, Pancreatic Cancer, Pancreatic Neuroendocrine Tumor (NET), Penile Cancer, Pituitary Tumors, Prostate Cancer, Retinoblastoma, Rhabdomyosarcoma, Salivary Gland Cancer, Skin Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer, Waldenstrom Macroglobulinemia and Wilms Tumor.

In certain embodiments, the method comprises treating a cancer with a FANCM blocking agent. In particular embodiments, the cancer comprises an impaired, defective or deregulated DNA repair pathway. In certain embodiments, the DNA repair pathway is the homologous recombination (HR) repair pathway.

In some embodiments of the methods of the disclosure, the cancer is a BRCA negative (BRCA^{-/-}) ovarian cancer, e.g., a BRCA1^{-/-} and/or BRCA2^{-/-} ovarian cancer. In some embodiments, the BRCA is BRCA1 and/or BRCA2. Thus, in certain embodiments, target cells are BRCA1^{-/-} and/or BRCA2^{-/-}. In particular embodiments, the BRCA1 and/or BRCA2 gene comprises a deletion or modification. In some embodiments, the BRCA^{-/-} ovarian cancer is resistant to treatment with a PARP inhibitor. In some embodiments, the BRCA^{-/-} ovarian cancer resistance to a PARP inhibitor is acquired resistance following treatment with a PARP inhibitor. In certain embodiments, the disclosure provides a method for treating a BRCA^{-/-} ovarian cancer in a subject, comprising providing to the subject a FANCM blocking agent. In particular embodiments, the blocking agent is an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

In some embodiments of the methods of the disclosure, the cancer is a BRCA negative (BRCA^{-/-}) breast cancer, e.g., a BRCA1^{-/-} and/or BRCA2^{-/-} breast cancer. In some embodiments, the BRCA is BRCA1 and/or BRCA2. In particular embodiments, the BRCA1 and/or BRCA2 gene comprises a deletion or modification. In some embodiments, the BRCA^{-/-} breast cancer is resistant to treatment with a PARP inhibitor. In some embodiments, the BRCA^{-/-} breast cancer resistance to a PARP inhibitor is acquired resistance following treatment with a PARP inhibitor. In certain embodiments, the disclosure provides a method for treating a BRCA^{-/-} breast cancer in a subject, comprising providing to the subject a FANCM blocking agent. In particular embodiments, the blocking agent is an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

In some embodiments of the methods of the disclosure, the target cell is in vivo or ex vivo. In some embodiments, compositions of the disclosure are contacted to a target cell in vivo or administered to a subject comprising the target cell to treat a disease or disorder of the disclosure. In some embodiments, compositions of the disclosure are contacted to a target cell ex vivo. The target cell may be cultured or maintained as a culture for use as a companion diagnostic. The target cell may be contacted with a composition of the disclosure ex vivo to confirm the subject's response to a therapeutic administration of the composition prior to the therapeutic administration. The target cell may be used to develop a library of cells for screening compositions of the disclosure as a companion diagnostic method.

In some embodiments of the methods of the disclosure, the method comprises administrating to a subject an effective amount of the composition of the disclosure. In some embodiments, the method further comprises administering a second therapy or a second therapeutic composition to the subject. In some embodiments, the second therapy or the second therapeutic composition is administered simultaneously with a composition of the disclosure. In some embodiments, the second therapy or the second therapeutic composition is administered sequentially with a composition of the disclosure. In some embodiments, a composition of the disclosure is administered to subject and, subsequently, a second therapy or a second therapeutic composition is administered to the subject. In some embodiments, a second therapy or a second therapeutic composition is administered to the subject, and subsequently, a composition of the disclosure is administered to the subject. In some embodiments, a therapeutically effective amount of a second therapy or a second therapeutic composition is administered to the subject

In some embodiments of the methods of the disclosure, a second therapy or a second therapeutic composition comprises a radiation treatment and/or a chemotherapy. In some embodiments, the chemotherapy comprises a Poly (ADP-ribose) polymerase (PARP) inhibitor or a platinum-based therapy. In some embodiments, the disease or disorder is cancer and the cancer is resistant to treatment with a PARP inhibitor as a monotherapy. In some embodiments, prior to administration of the composition, the subject has been identified as resistant to treatment with a PARP inhibitor as a monotherapy. In some embodiments, prior to administration of the composition, the subject has been treated with a PARP inhibitor as a monotherapy.

In some embodiments of the methods of the disclosure, the administration of a composition of the disclosure or of a second therapy or of a second therapeutic composition is systemic. In some embodiments, a composition of the disclosure is administered by one or more of an oral route, an inhaled route, an intravenous route, an intraperitoneal route, and a subcutaneous route. In some embodiments, a second therapy or a second therapeutic composition of the disclosure is administered by one or more of an oral route, an inhaled route, an intravenous route, an intraperitoneal route, and a subcutaneous route.

In some embodiments of the methods of the disclosure, the administration of a composition of the disclosure or of a second therapy or of a second therapeutic composition is local. In some embodiments, a composition of the disclosure is administered by one or more of an intraocular route, an intraspinal route, an intracerebellar route, an intrathecal route, an intramuscular route and an intraosseous route. In some embodiments, a second therapy or a second therapeutic composition of the disclosure is administered by one or more of an intraocular route, an intraspinal route, an intracerebellar route, an intrathecal route, an intramuscular route and an intraosseous route.

In some embodiments of the methods of the disclosure, a composition of the disclosure or of a second therapy or of a second therapeutic composition is administered once per day, twice per day or three times per day. In some embodiments of the methods of the disclosure, a composition of the disclosure or of a second therapy or of a second therapeutic composition is administered once per week, twice per week or three times per week. In some embodiments of the methods of the disclosure, a composition of the disclosure or of a second therapy or of a second therapeutic composition is administered once per month, twice per month or three times per month.

In some embodiments of the methods of the disclosure, including those wherein the disease or disorder is cancer, treating comprises a reduction in a severity of a sign or symptom of the cancer. In some embodiments, a therapeutically effective amount of a composition of the disclosure or of a second therapy or of a second therapeutic composition reduces the severity of a sign or symptom of the cancer.

In some embodiments of the methods of the disclosure, including those wherein the disease or disorder is cancer, treating comprises a reduction in a volume of a tumor. In some embodiments, a therapeutically effective amount of a composition of the disclosure or of a second therapy or of a second therapeutic composition reduces a number of tumor cells per volume of blood or mass of tissue.

In some embodiments of the methods of the disclosure, including those wherein the disease or disorder is cancer, treating comprises a remission. In some embodiments, a therapeutically effective amount of a composition of the disclosure or of a second therapy or of a second therapeutic composition induces a remission.

In some embodiments of the methods of the disclosure, including those wherein the disease or disorder is cancer, treating comprises an increased duration of progression free survival. In some embodiments, a therapeutically effective amount of a composition of the disclosure or of a second therapy or of a second therapeutic composition increases the duration of progression free survival.

### Blocking Agents

Blocking agents may selectively inhibit or disrupt one or more DNA repair pathways or components thereof. In certain embodiments, a blocking agent inhibits or disrupts LIG1 or FANCM.

In certain embodiments, the blocking agent specifically, selectively or preferentially binds to or inhibits a targeted DNA repair pathway or component thereof. In certain embodiments, the blocking agent binds to the target with a dissociation constant (KD) of about 1 μM or less, about 100 nM or less, about 40 nM or less, about 20 nM or less, or about 10 nM or less.

Various types of blocking agents are known in the art and may be used according to aspects of the disclosure. In particular embodiments, a blocking agent is an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

In certain embodiments, the blocking agent is a small molecule, e.g., a small organic molecule that inhibits a target DNA repair pathway or component thereof, e.g., LIG1 or FANCM.

In certain embodiments, the blocking agent is a polypeptide. In particular embodiments, a blocking agent comprises a variant of a targeted DNA repair pathway protein, such as, e.g. a LIG1 or FANCM variant or fragment, or fragment variant, including but not limited to any disclosed herein. In particular embodiments, the variant comprises a mutation in one or more amino acids required for a function or activity of the targeted DNA repair pathway protein. In certain embodimemts, the variant is a fragment of the targeted DNA repair pathway protein or variant thereof, e.g., a dominant negative inhibitor.

In certain embodiments, the blocking agent is an antibody or functional fragment thereof, which binds to a targeted DNA repair pathway protein, e.g., LIG1 or FANCM, to inhibit one or more functions or activities of the targeted protein. A variety of antibodies and functional fragments thereof are known in the art and may be used according to the disclosure, including but not limited to monoclonal antibodies. The term “monoclonal antibody” encompasses not only intact monoclonal antibodies and full-length monoclonal antibodies, but also fragments thereof (such as Fab, Fab′, F(ab′)2, Fv), single chain Fv (scFv), diabodies, single domain (sdAb) or VHH antibody fragment (also known as a Nanobody®), UniBody®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

In certain embodiments, a blocking agent is a component of a gene editing system. As used herein, a gene-editing system is a system comprising one or more proteins or polynucleotides capable of editing an endogenous target gene or locus in a sequence specific manner. In some embodiments, the gene-editing system is a protein-based gene regulating system comprising a protein comprising one or more zinc-finger binding domains and an enzymatic domain. In some embodiments, the protein-based gene regulating system comprises a protein comprising a Transcription activator-like effector nuclease (TALEN) domain and an enzymatic domain. Such embodiments are referred to herein as “TALENs”.

Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus in a target gene referenced in Table 1), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

TALEN-based systems comprise a protein comprising a TAL effector DNA binding domain and an enzymatic domain. They are made by fusing a TAL effector DNA-binding domain to a DNA cleavage domain (a nuclease which cuts DNA strands). The FokI restriction enzyme is an exemplary enzymatic domain suitable for use in TALEN-based gene regulating systems. Methods and compositions for assembling TAL-effector repeats are known in the art. See e.g., Cermak et al, Nucleic Acids Research, 39:12, 2011, e82. Plasmids for constructions of the TAL-effector repeats are commercially available from Addgene.

In some embodiments, the gene-editing system is a combination gene-regulating system comprising a site-directed modifying polypeptide and a nucleic acid guide molecule. Herein, a “site-directed modifying polypeptide” refers to a polypeptide that binds to a nucleic acid guide molecule, is targeted to a target nucleic acid sequence, such as, for example, a DNA sequence, by the nucleic acid guide molecule to which it is bound, and modifies the target DNA sequence (e.g., cleavage, mutation, or methylation of target DNA). A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA cleavage, histone acetylation, histone methylation, etc.), wherein the site of enzymatic activity is determined by the guide nucleic acid.

The nucleic acid guide comprises two portions: a first portion that is complementary to, and capable of binding with, an endogenous target DNA sequence (referred to herein as a “DNA-binding segment”), and a second portion that is capable of interacting with the site-directed modifying polypeptide (referred to herein as a “protein-binding segment”). In some embodiments, the DNA-binding segment and protein-binding segment of a nucleic acid guide are comprised within a single polynucleotide molecule. In some embodiments, the DNA-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic gene regulating systems by specifically hybridizing with a target DNA sequence comprised within the DNA sequence of a target gene. Reference herein to a target gene encompasses the full-length DNA sequence for that particular gene and a full-length DNA sequence for a particular target gene will comprise a plurality of target genetic loci, which refer to portions of a particular target gene sequence (e.g., an exon or an intron). Within each target genetic loci are shorter stretches of DNA sequences referred to herein as “target DNA sequences” or “target sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification). The gene-regulating systems described herein may comprise a single nucleic acid guide, or may comprise a plurality of nucleic acid guides (e.g., 2, 3, 4, 5, 6, 7, 8, 9, 10, or more nucleic acid guides).

In some embodiments, the gene editing systems described herein are CRISPR (Clustered Regularly Interspaced Short Palindromic Repeats)/Cas (CRISPR Associated) nuclease systems. In such embodiments, the site-directed modifying polypeptide is a CRISPR-associated endonuclease (a “Cas” endonuclease) and the nucleic acid guide molecule is a guide RNA (gRNA).

A Cas polypeptide refers to a polypeptide that can interact with a gRNA molecule and, in concert with the gRNA molecule, homes or localizes to a target DNA sequence and includes naturally occurring Cas proteins and engineered, altered, or otherwise modified Cas proteins that differ by one or more amino acid residues from a naturally-occurring Cas sequence.

In some embodiments, the Cas protein is a Cas9 protein. Cas9 is a multi-domain enzyme that uses an HNH nuclease domain to cleave the target strand of DNA and a RuvC-like domain to cleave the non-target strand. In some embodiments, mutants of Cas9 can be generated by selective domain inactivation enabling the conversion of WT Cas9 into an enzymatically inactive mutant (e.g., dCas9), which is unable to cleave DNA, or a nickase mutant, which is able to produce single-stranded DNA breaks by cleaving one or the other of the target or non-target strand.

A guide RNA (gRNA) comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein.

The DNA-binding segment (or “DNA-binding sequence”) of a gRNA comprises a nucleotide sequence that is complementary to and capable of binding to a specific sequence target DNA sequence. The protein-binding segment of the gRNA interacts with a Cas polypeptide and the interaction of the gRNA molecule and site-directed modifying polypeptide results in Cas binding to the endogenous DNA and produces one or more modifications within or around the target DNA sequence. The precise location of the target modification site is determined by both (i) base-pairing complementarity between the gRNA and the target DNA sequence; and (ii) the location of a short motif, referred to as the protospacer adjacent motif (PAM), in the target DNA sequence. The PAM sequence is required for Cas binding to the target DNA sequence. A variety of PAM sequences are known in the art and are suitable for use with a particular Cas endonuclease (e.g., a Cas9 endonuclease) are known in the art (See e.g., Nat Methods. 2013 November; 10(11): 1116-1121 and Sci Rep. 2014; 4: 5405). In some embodiments, the PAM sequence is located within 50 base pairs of the target modification site. In some embodiments, the PAM sequence is located within 10 base pairs of the target modification site. The DNA sequences that can be targeted by this method are limited only by the relative distance of the PAM sequence to the target modification site and the presence of a unique 20 base pair sequence to mediate sequence-specific, gRNA-mediated Cas binding. In some embodiments, the target modification site is located at the 5′ terminus of the target locus. In some embodiments, the target modification site is located at the 3′ end of the target locus. In some embodiments, the target modification site is located within an intron or an exon of the target locus.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

In some embodiments, the site-directed modifying polypeptide is a Cas protein. Cas molecules of a variety of species can be used in the methods and compositions described herein.

In some embodiments, the Cas protein is a Cas9 protein or a Cas9 ortholog.

Guide RNAs (gRNAs) direct a site-directed modifying polypeptide to a specific target DNA sequence. A gRNA comprises a DNA-targeting segment and protein-binding segment. The DNA-targeting segment of a gRNA comprises a nucleotide sequence that is complementary to a sequence in the target DNA sequence. As such, the DNA-targeting segment of a gRNA interacts with a target DNA in a sequence-specific manner via hybridization (i.e., base pairing), and the nucleotide sequence of the DNA-targeting segment determines the location within the target DNA that the gRNA will bind. The DNA-targeting segment of a gRNA can be modified (e.g., by genetic engineering) to hybridize to any desired sequence within a target DNA sequence.

The protein-binding segment of a guide RNA interacts with a site-directed modifying polypeptide (e.g. a Cas9 protein) to form a complex. The guide RNA guides the bound polypeptide to a specific nucleotide sequence within target DNA via the above-described DNA-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single RNA molecule (sgRNA). The specificity of a gRNA for a target loci is mediated by the sequence of the DNA-binding segment, which comprises about 20 nucleotides that are complementary to a target DNA sequence within the target locus. In some embodiments, the corresponding target DNA sequence is approximately 20 nucleotides in length. In some embodiments, the DNA-binding segments of the gRNA sequences of the present invention are at least 90% complementary to a target DNA sequence within a target locus. In some embodiments, the DNA-binding segments of the gRNA sequences of the present invention are at least 95%, 96%, 97%, 98%, or 99% complementary to a target DNA sequence within a target locus. In some embodiments, the DNA-binding segments of the gRNA sequences of the present invention are 100% complementary to a target DNA sequence within a target locus.

In some embodiments, the DNA-binding segments of the gRNA sequences bind to a target DNA sequence that is at least 90% identical to a target DNA sequence of a target disclosed herein. In some embodiments, the DNA-binding segments of the gRNA sequences bind to a target DNA sequence that is at least 95%, 96%, 97%, 98%, or 99% identical to a target DNA sequence disclosed herein for LIG1 or FANCM. In some embodiments, the DNA-binding segments of the gRNA sequences bind to a target DNA sequence that is 100% identical to a target DNA sequence within a target locus of a LIG1 or FANCM gene.

### Enumerated Embodiments

Certain non-limiting embodiments of aspects of the disclosure are enumerated below.

1. A composition comprising a Fanconi Anemia Group M protein (FANCM) blocking agent, wherein in a target cell comprising an impaired, defective or deregulated DNA repair pathway, the blocking agent reduces or inhibits a function of FANCM.
2. The composition of 1, wherein the target cell is a proliferating cell.
3. The composition of 1 or 2, wherein the target cell is a tumor cell.
4. The composition of any one of 1-3, wherein the target cell is a malignant cell.
5. The composition of any one of 1-4, wherein the target cell is a metastatic cell.
6. The composition of any one of 1-5, wherein the impaired, defective or deregulated DNA repair pathway is an impaired, defective or deregulated homologous recombination (HR) repair pathway, optionally wherein the target cell comprises a variant protein of the HR pathway.
7. The composition of 6, wherein the target cell comprises a BRCA gene deletion, a variant BRCA protein, or a sequence encoding a variant BRCA protein, wherein the variant BRCA protein induces a loss or reduction in a function of the HR pathway.
8. The composition of 7, wherein the BRCA gene deletion is a BRCA1 gene deletion, and the variant BRCA protein comprises a variant BRCA1 protein, or wherein the sequence encoding the variant BRCA protein comprises a sequence encoding a variant BRCA1 protein.
9. The composition of 7, wherein the BRCA gene deletion is a BRCA2 gene deletion, and the variant BRCA protein comprises a variant BRCA2 protein, or wherein the sequence encoding the variant BRCA protein comprises a sequence encoding a variant BRCA2 protein.
10. The composition of 6, wherein the target cell comprises a nucleic acid or an amino acid encoding a variant DNA repair protein RAD51 homolog 1 (RAD51) or a variant homolog of RAD51 and wherein the variant RAD51 induces a loss or reduction in a function of the HR pathway.
11. The composition of 6, wherein the target cell comprises a nucleic acid or an amino acid encoding a variant DNA repair protein RAD51 homolog 3 (RAD51C) or a variant homolog of RAD51C and wherein the variant RAD51C induces a loss or reduction in a function of the HR pathway.
12. The composition of 6, wherein the target cell comprises a nucleic acid or an amino acid encoding a variant DNA repair protein RAD51 homolog 4 (RAD51D) or a variant homolog of RAD51D and wherein the variant RAD51D induces a loss or reduction in a function of the HR pathway.
13. The composition of 6, wherein the target cell comprises a nucleic acid or an amino acid encoding a variant X-ray repair cross-complementing 2 (XRCC2) or a variant homolog of XRCC2 and wherein the variant XRCC2 induces a loss or reduction in a function of the HR pathway.
14. The composition of 6, wherein the target cell comprises a nucleic acid or an amino acid encoding a variant DNA repair endonuclease XPF or a variant homolog of XPF and wherein the variant XPF induces a loss or reduction in a function of the HR pathway.
15. The composition of 6, wherein the target cell comprises a nucleic acid or an amino acid encoding a variant Meiotic recombination 11 homolog 1 (MRE11A) or a variant homolog of MRE11A and wherein the variant MRE11A induces a loss or reduction in a function of the HR pathway.
16. The composition of 6, wherein the target cell comprises a nucleic acid or an amino acid encoding a variant Ataxia telangiectasia mutated (ATM) or a variant homolog of ATM and wherein the variant ATM induces a loss or reduction in a function of the HR pathway.
17. The composition of 6, wherein the target cell comprises a nucleic acid or an amino acid encoding a variant BRCA1-associated RING domain protein 1 (BARD1) or a variant homolog of BARD1 and wherein the variant BARD1 induces a loss or reduction in a function of the HR pathway.
18. The composition of 6, wherein the target cell comprises a nucleic acid or an amino acid encoding a variant BRCA1-interacting protein C-terminal helicase 1 (BRIP1) or a variant homolog of BRIP1 and wherein the variant BRIP1 induces a loss or reduction in a function of the HR pathway.
19. The composition of 6, wherein the target cell comprises a nucleic acid or an amino acid encoding a variant Cell cycle checkpoint kinase (CHEK1) or a variant homolog of CHEK1 and wherein the variant CHEK1 induces a loss or reduction in a function of the HR pathway.
20. The composition of 6, wherein the target cell comprises a nucleic acid or an amino acid encoding a variant CHK1 checkpoint homolog (CHEK2) or a variant homolog of CHEK2 and wherein the variant CHEK2 induces a loss or reduction in a function of the HR pathway.
21. The composition of 6, wherein the target cell comprises a nucleic acid or an amino acid encoding a variant Nibrin (NBN) or a variant homolog of NBN and wherein the variant NBN induces a loss or reduction in a function of the HR pathway.
22. The composition of 6, wherein the target cell comprises a nucleic acid or an amino acid encoding a variant Partner and localizer of BRCA2 (PALB2) or a variant homolog of PALB2 and wherein the variant PALB2 induces a loss or reduction in a function of the HR pathway.
23. The composition of 6, wherein the target cell comprises a nucleic acid or an amino acid encoding a variant Structure-specific endonuclease subunit SLX4 (SLX4) or a variant homolog of SLX4 and wherein the variant SLX4 induces a loss or reduction in a function of the HR pathway.
24. The composition of any one of 7-23, wherein the variant protein or the sequence encoding the variant protein comprises one or more of a mutation, a deletion, a promotor methylation, a silencing event and a splicing event.
25. The composition of 24, wherein the mutation comprises one or more of a substitution, an insertion, a deletion, an inversion, and a translocation of a nucleic acid sequence or an amino acid sequence encoding the variant BRCA protein, optionally wherein the mutation is BRCA1 or BRCA2 gene deletion, or wherein the variant BRCA protein is a variant BRCA1 protein or a variant BRCA2 protein.
26. The composition of 24 or 25, wherein the mutation introduces a stop codon into a nucleic acid sequence encoding the variant protein, thereby generating one or more of a truncated protein, an inactivated protein and a protein fragment.
27. The composition of any one of 3-28, wherein the variant protein or the sequence encoding the variant protein comprises a promoter sequence, and wherein the promoter controls expression of the variant protein or the sequence encoding the variant protein.
28. The composition of 27, wherein the silencing event comprises a silencing of the promoter sequence, function, or activity.
29. The composition of 27 or 28, wherein the promoter sequence controlling expression of the variant protein or the sequence encoding the variant protein comprises a mutation.
30. The composition of 29, wherein the mutation comprises one or more of a substitution, an insertion, a deletion, an inversion, and a translocation of the promoter sequence.
31. The composition of any one of 1-30, wherein the function of FANCM comprises one or more of ATP-binding, nucleotide-binding, DNA-binding, DNA remodeling, DNA strand separation, DNA-RNA strand separation and catalyzing the break of a chemical bond using water.
32. The composition of any one of 1-30, wherein the function of FANCM comprises a helicase activity.
33. The composition of any one of 1-30, wherein the function of FANCM comprises a hydrolase activity.
34. The composition of any one of 1-30, wherein the function of FANCM comprises a translocase activity.
35. The composition of any one of 1-34, wherein the function of FANCM comprises an ATPase activity.
36. The composition of any one of 6-35, wherein the variant protein increases a function of FANCM.
37. The composition of any one of 6-35, wherein the variant protein decreases a function of FANCM.
38. The composition of any one of 1-30, wherein the impairment, defect or deregulation of the HR pathway increases a dependence of the target cell upon a function of FANCM.
39. The composition of any one of 6-38, wherein the function of the HR pathway comprises one or more of
i) recognizing nucleotide or DNA damage;
ii) recruiting a protein to a site of nucleotide or DNA damage;
iii) configuring or remodeling a sequence comprising a site of nucleotide or DNA damage;
iv) configuring or remodeling a sequence complementary to a site of nucleotide or DNA damage;
v) inducing a break in a sequence within a site of nucleotide or DNA damage;
vi) inducing a break in a sequence comprising the site of nucleotide or DNA damage;
vii) inducing a break in a sequence complementary to a site of nucleotide or DNA damage;
viii) removing a sequence within a site of nucleotide or DNA damage;
ix) removing a sequence comprising a site of nucleotide or DNA damage;
x) synthesizing a new sequence within a site of nucleotide or DNA damage;
xi) synthesizing a new sequence comprising a site of nucleotide or DNA damage;
xii) resecting a portion of a synthesized sequence within a site of nucleotide or DNA damage;
xiii) resecting a portion of a synthesized sequence comprising the site of nucleotide or DNA damage;
xiv) stabilizing a site of DNA synthesis or replication within a site of nucleotide or DNA damage;
xv) stabilizing a site of DNA synthesis or replication comprising a site of nucleotide or DNA damage;
xvi) stabilizing a site of DNA synthesis or replication comprising a target site;
xvii) stabilizing a site of DNA synthesis or replication comprising a stalled replication fork;
xviii) inducing or facilitating invasion of a synthesized sequence within the site of nucleotide or DNA damage;
xix) inducing or facilitating invasion of a synthesized sequence comprising the site of nucleotide or DNA damage;
xx) inducing or facilitating insertion of a synthesized sequence within the site of nucleotide or DNA damage by recombination; and
xxi) inducing or facilitating insertion of a synthesized sequence comprising the site of nucleotide or DNA damage by recombination.
40. The composition of any one of 6-39, wherein an activity of the HR pathway comprises an increase or a decrease in a function of a component of the HR pathway.
41. The composition of any one of 6-40, wherein the variant protein increases a function of a component of the HR pathway.
42. The composition of any one of 6-40, wherein the variant protein decreases a function of a component of the HR pathway.
43. The composition of any one of 1-42, further comprising a pharmaceutically-acceptable carrier.
44. The composition of any one of 1-43, wherein the blocking agent comprises an effector moiety that binds to a FANCM protein or a nucleic acid sequence encoding the FANCM protein.
45. The composition of 44, wherein the effector moiety comprises one or more of an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.
46. The composition of any one of 1-45, wherein the blocking agent further comprises a targeting moiety operably-linked to the effector moiety.
47. The composition of 46, wherein the targeting moiety is reversibly-linked to the effector moiety.
48. The composition of 46 or 47, wherein the targeting moiety specifically binds a component of the target cell.
49. The composition of any one of 1-48, wherein the target cell is a proliferating cell.
50. The composition of any one of 1-49, wherein the target cell is a tumor cell.
51. The composition of any one of 1-50, wherein the target cell is a malignant cell.
52. The composition of any one of 1-51, wherein the target cell is a metastatic cell.
53. The composition of any one of 1-52, wherein the target cell is produced or derived from a non-hematological tissue.
54. The composition of any one of 1-53, wherein the target cell is produced or derived from an epithelial tissue.
55. The composition of any one of 1-54, wherein the target cell is produced or derived from an organ or a structure comprising an epithelial tissue.
56. The composition of any one of 1-55, wherein the target cell is produced or derived from a skin area, a skin layer, a lung, a lymph node, a breast, an ovary, a prostate, a mouth, a nose, a nasal passage, an esophagus, an intestine, a small intestine, a large intestine, a stomach, a kidney, a liver, a spleen, a heart, an artery, a vein, a bladder and a colon.
57. The composition of any one of 1-52, wherein the target cell is produced or derived from a bone or a connective tissue.
58. The composition of any one of 1-57, wherein the blocking agent further comprises a regulation moiety.
59. The composition of 58, wherein the regulation moiety is operably-linked to one or more of the effector moiety and the targeting moiety.
60. The composition of 58 or 59, wherein the regulation moiety is reversibly-linked to one or more of the effector moiety and the targeting moiety.
61. The composition of any one of 58-60, wherein the regulation moiety selectively binds a component not present in a target cell.
62. The composition of 61, wherein the component not present in a target cell is present in a healthy cell.
63. The composition of 61 or 62, wherein the component decreases or inhibits an activity of the effector moiety.
64. The composition of any one of 58-63, wherein the regulation moiety comprises a microRNA (miRNA) binding site and selectively binds a miRNA.
65. The composition of any one of 1-64, wherein the target cell is an ALT+ cell.
66. The composition of any one of 1-64, wherein the target cell is not an ALT+ cell.
67. A method of inducing cell death in a proliferating cell, comprising contacting the proliferating cell with the composition of any one of 1-66.
68. The method of 67, wherein the cell is in vitro or ex vivo.
69. The method of 67, wherein the cell is in vivo.
70. A method of inducing cell cycle arrest in a proliferating cell, comprising contacting the proliferating cell with the composition of any one of 1-66.
71. The method of 70, wherein the proliferating cell is in vitro or ex vivo.
72. The method of 71, wherein the proliferating cell is in vivo.
73. The method of any one of 67-72, wherein the proliferating cell comprises the impaired, defective or deregulated DNA repair pathway.
74. The method of 73, wherein the proliferating cell comprises the impaired, defective or deregulated homologous recombination (HR) repair pathway.
75. The method of 74, wherein the proliferating cell comprises a variant BRCA protein or a sequence encoding a variant BRCA protein, and wherein the variant BRCA protein induces a loss or reduction in a function of the HR pathway.
76. The method of 75, wherein the variant BRCA protein comprises a variant BRCA1 protein or a variant BRCA2 protein, or wherein the sequence encoding the variant BRCA protein comprises a sequence encoding a variant BRCA1 protein or a variant BRCA2 protein.
77. The method of any one of 70-76, wherein the proliferating cell is an ALT+ cell.
78. The method of any one of 70-76, wherein the proliferating cell is not an ALT+ cell.
79. The method of any one of 70-78, wherein the proliferating cell is resistant to a PARP inhibitor.
80. The method of any one of 70-79, wherein the proliferating cell is an ovarian tumor cell or a breast tumor cell, optionally a BRCA1−/− tumor cell or a tumor cell comprising the variant BRCA1 protein, or a sequence encoding the variant BRCA protein.
81. A method of treating cancer, comprising administrating to a subject an effective amount of the composition of any one of 1-66.
82. The method of 81, wherein the method further comprises administering a second therapy.
83. The method of 82, wherein the second therapy comprises radiation and/or a chemotherapy.
84. The method of 83, wherein the chemotherapy comprises a Poly (ADP-ribose) polymerase (PARP) inhibitor or a platinum-based therapy.
85. The method of any one of 81-84, wherein the cancer is resistant to treatment with a PARP inhibitor as a monotherapy.
86. The method of any one of 81-85, wherein, prior to administration of the composition, the subject has been identified as resistant to treatment with a PARP inhibitor as a monotherapy.
87. The method of any one of 81-86, wherein, prior to administration of the composition, the subject has been treated with a PARP inhibitor as a monotherapy.
88. The method of any one of 81-87, wherein the administration is systemic.
89. The method of 88, wherein the composition is administered by one or more of an oral route, an inhaled route, an intravenous route, an intraperitoneal route, and a subcutaneous route.
90. The method of any one of 81-87, wherein the administration is local.
91. The method of 90, wherein the composition is administered by one or more of an intraocular route, an intraspinal route, an intracerebellar route, an intrathecal route, an intramuscular route and an intraosseous route.
92. The method of any one of 81-91, wherein the composition is administered once per day, twice per day or three times per day.
93. The method of any one of 81-92, wherein the composition is administered once per week, twice per week or three times per week.
94. The method of any one of 81-93, wherein the composition is administered once per month, twice per month or three times per month.
95. The method of any one of 81-94, wherein treating comprises a reduction in a severity of a sign or symptom of the cancer.
96. The method of any one of 81-95, wherein treating comprises a reduction in a volume of a tumor.
97. The method of any one of 81-96, wherein treating comprises a reduction in a number of tumor cells per volume of blood or mass of tissue.
98. The method of any one of 81-97, wherein treating comprises a remission.
99. The method of any one of 81-98, wherein treating comprises an increased duration of progression free survival.
100. The method of any one of 81-99, wherein the cancer comprises cancer cells comprising an impaired, defective or deregulated DNA repair pathway.
101. The method of 100, wherein the cancer cells comprise an impaired, defective or deregulated homologous recombination (HR) repair pathway.
102. The method of 101, wherein the cancer cells comprise a variant BRCA protein or a sequence encoding a variant BRCA protein, and wherein the variant BRCA protein induces a loss or reduction in a function of the HR pathway.
103. The method of 102, wherein the variant BRCA protein comprises a variant BRCA1 protein or a variant BRCA2 protein, or wherein the sequence encoding the variant BRCA protein comprises a sequence encoding a variant BRCA1 protein or a variant BRCA2 protein.
104. The method of any one of 81-103, wherein the cancer cells are ALT+ cells.
105. The method of any one of 81-103, wherein the cancer cells are not an ALT+ cells.
106. The method of any one of 81-105, wherein the cancer cells are resistant to a PARP inhibitor.
107. The method of any one of 81-106, wherein the cancer is an ovarian cancer or a breast cancer, optionally a BRCA1−/− cancer or a cancer comprising tumor cells comprising a variant BRCA1 protein, or a sequence encoding the variant BRCA protein.
108. The method of 81, wherein the method comprises administering to a subject having a BRCA1−/− ovarian cancer or BRCA1−/− breast cancer a Fanconi Anemia Group M protein (FANCM) blocking agent.
109. The method of 108, wherein the method results in DNA damage and/or cell cycle arrest of BRCA1−/− ovarian cancer cells or BRCA1−/− breast cancer cells.
110. The method of 108 or 109, wherein the method decreases survival or growth of BRCA1−/− ovarian cancer cells or BRCA1−/− breast cancer cells.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention. Accordingly, the invention is not limited except as by the appended claims.

Furthermore, it is intended that any method described in the disclosure may be rewritten into Swiss-type format for the use of any agent described in the disclosure, for the manufacture of a medicament, in treating any of the disorders described in the disclosure. Likewise, it is intended for any method described in the disclosure to be rewritten as a compound for use claim, or as a use of a compound claim.

All U.S. patents, U.S. patent application publications, U.S. patent applications, foreign patents, foreign patent applications and non-patent publications referred to in this specification are incorporated herein by reference, in their entirety.

### EXAMPLES

The disclosure is further illustrated by the following examples, which are not to be construed as limiting this disclosure in scope or spirit to the specific procedures described in the disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure.

### Example 1

### Crispr/CAS9-Mediated Knockout of FANCM in BRCA1−/− Cancer Cell Lines Leads to an Increase in DNA Damage and G2 Arrest and a Reduction of Growth in 2D Clonogenic Assay

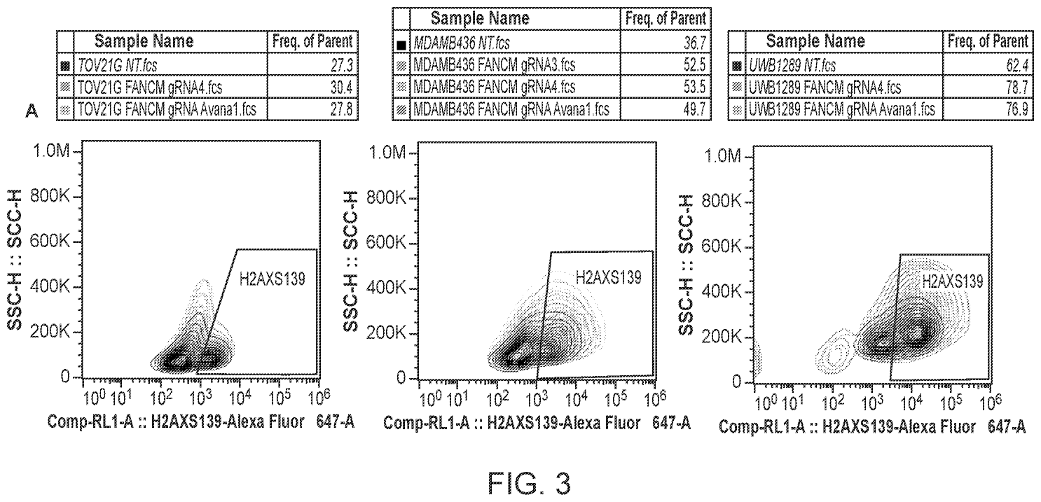

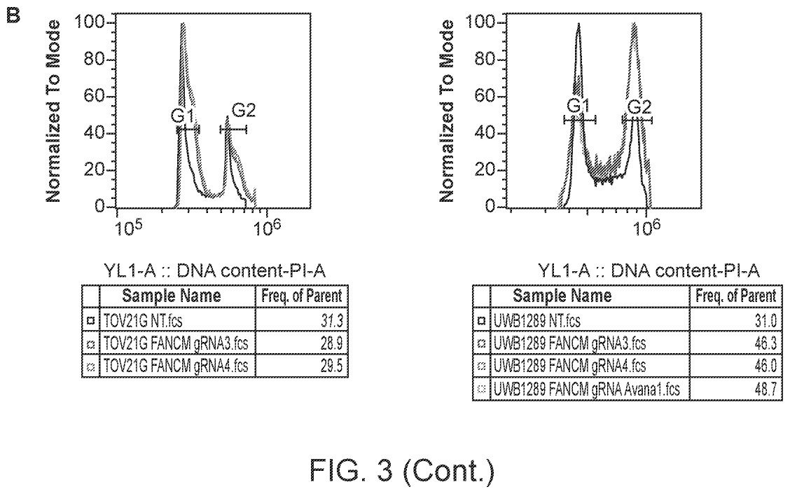

To determine the molecular consequences of FANCM loss on a BRCA1-null background, we performed CRISPR/Cas9 knockout experiments in two BRCA1−/− cancer cell lines: MDAMD436 (breast) and UWB1289 (ovarian) as well as a BRCA1-proficient ovarian cancer control: TOV21G. We designed three different sgRNAs to target the FANCM locus: gRNA3, gRNA4, gRNA Avana1, and harvested cells on Day 7 post CRISPR RNP nucleofection to measure DNA damage by flow cytometry for DNA double strand break marker pH2AXS139 (γH2AX). We observed a 13-17% increase in γH2AX-positive cells in both BRCA1−/− deficient cell lines MDAMB436 and UWB1289 in the conditions where FANCM was knocked out relative to the baseline levels with the NT control gRNA (FIG. 3A). In contrast, no significant changes in γH2AX-positive cells were observed when FANCM was knocked out in the BRCA1-proficient cell line TOV21G (FIG. 3A). We noted that the increase in DNA damage upon FANCM loss led to accumulation of cells in G2 phase of the cell cycle (FIG. 3B). As shown in FIG. 3B, while the percentage of TOV21G cells in G2 remained unchanged across experimental conditions (around 30%), there was a marked increase in the G2 population of UWB1289 cells in the FANCM knockout conditions (46-48%) relative to the NT control (around 30%). This increase of 16-18% in the G2 population tracks well with the 13-17% increase in γH2AX-positive cells, registered for the FANCM knockout conditions in the BRCA1−/− cell lines MDAMB436 and UWB1289 on FIG. 3A. Thus, loss of FANCM in BRCA1−/− breast and ovarian cancer cell lines leads to an increase in DNA damage and G2 arrest, while BRCA1-proficient cells remain unaffected.

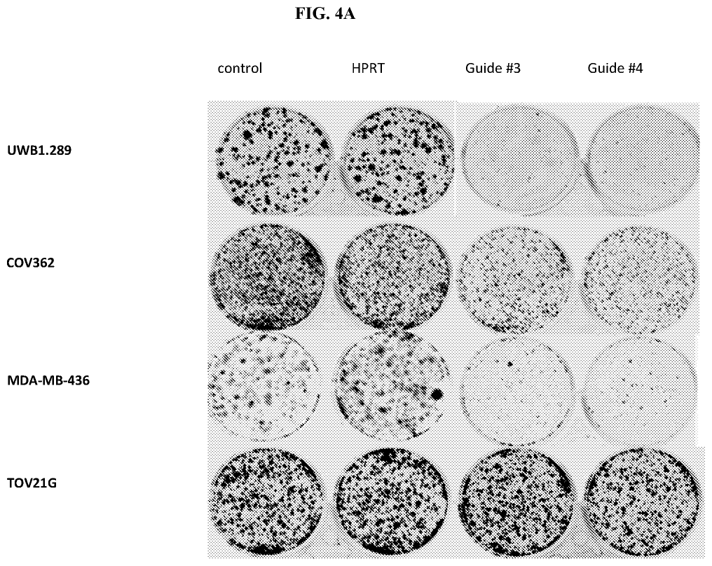

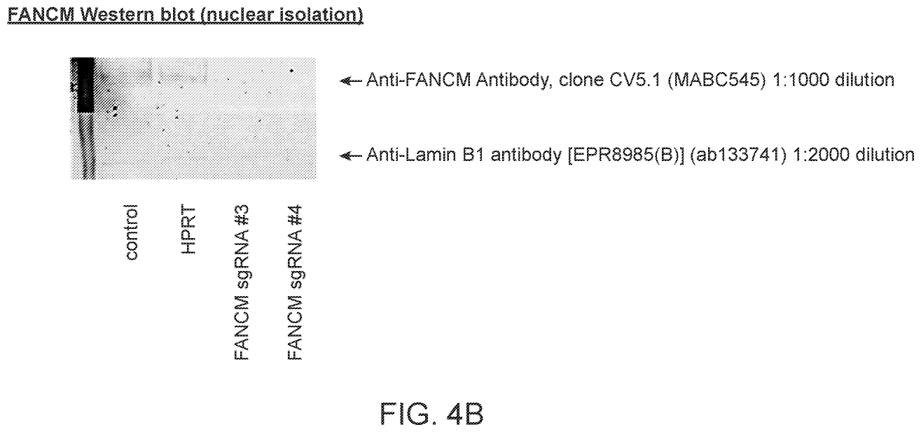

We also examined cell growth via a 2D colony formation assay on between 14-21 days post nucleofection with the CRISPR RNP targeting FANCM. As shown in FIG. 4A, CRISPR/Cas9-mediated knockout of FANCM in BRCA1−/− cancer cell lines, UWB1.289, COV362, and MDAMB436, led to a reduction of growth, which was not observed in the BRCA1 wild-type TOV21G cells. FANCM protein levels were determined by Western blot, and FIG. 4B shows loss of FANCM protein expression when cells were treated with the FANCM sgRNA #3 and #4.

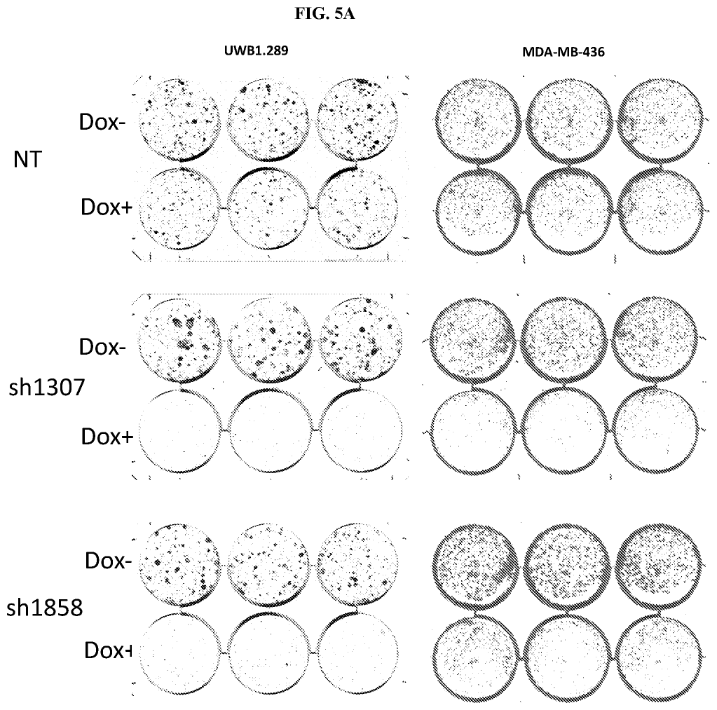

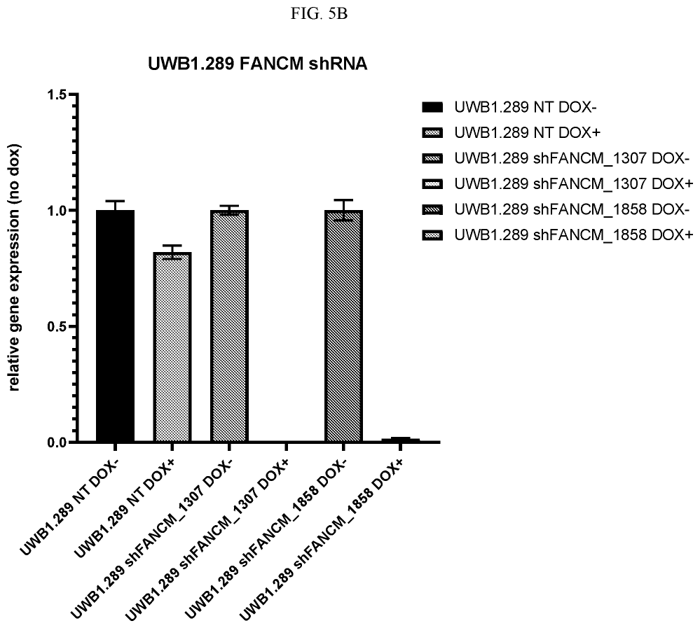

We further examined cell growth via the 2D colony formation assay on between days 14-21 following doxycycline induced shRNA expression of shRNA targeting FANCM for knockdown. As shown in FIG. 5A, shRNA knockdown of FANCM in BRCA1−/− cancer cell lines, UWB1.289 and MDAMB436, led to a reduction of growth. qPCR was performed to measure FANCM mRNA expression, and FIG. 5B shows loss of FANCM mRNA transcripts in UWB1.289 treated with shRNA #1307 and #1858 following three days of doxycline treatment confirming the on-target activity of the shRNAs and specific FANCM mRNA knockdown.

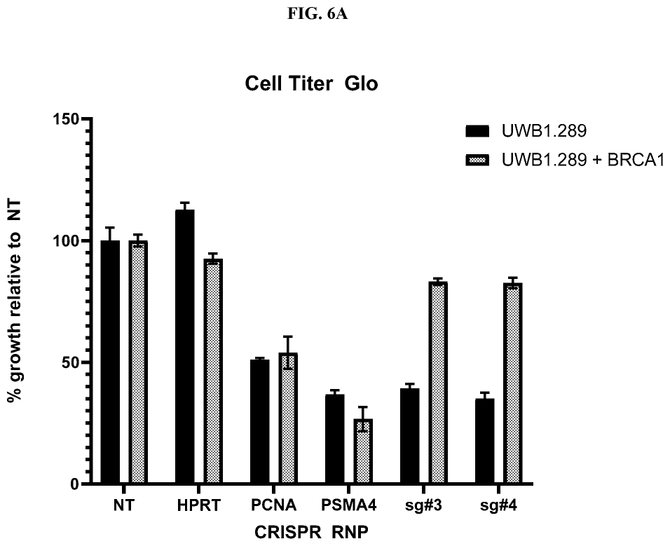

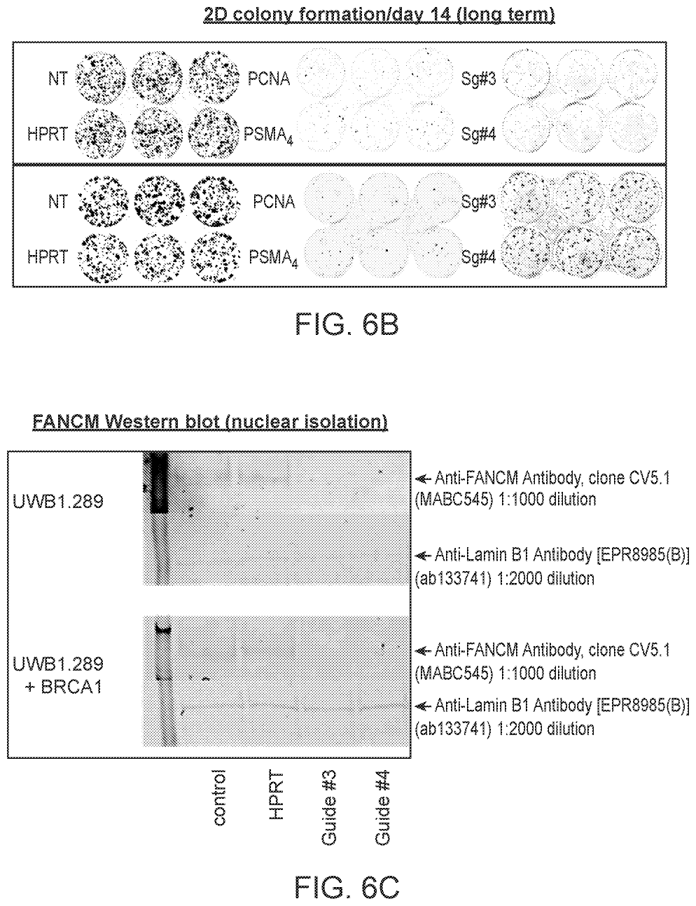

Short term and long term viability assays were conducted in BRCA1−/− cancer cell line UWB1.289 subjected to CRISPR/Cas9-mediated knockout of FANCM. FIGS. 6A and 6B demonstrate rescue of FANCM dependency when wild-type BRCA1 is present in UWB1.289 cells. FANCM protein levels were also determined via western blot, and FIG. 6C shows that nuclear enriched FANCM is undetectable in both UW1.289 null and BRCA1 add-back lines. Together, these data demonstrate a dependency on FANCM activity in BRCA1−/− cancer cell lines of different lineages and indicate that FANCM activity in this context is not redundant with other helicases/translocases but required for resolution of stalled replication forks or DNA damage in the absence of a functional BRCA1 protein.

### Materials and Methods

### Flow Cytometry:

For pH2AXS139 (γH2AX) staining, cells (up to 500K) were harvested 7 days after nucleofection with CRISPR/Cas9 RNP targeting the FANCM locus and fixed in 4% PFA for 10 min at RT. Following a wash with PBS, cells were permeabilized with 2% FBS in PBST (PBS+0.1% Triton) for 30 min at RT. Staining was performed with a γH2AX-A647 antibody (CST #9720S) at 1:50 in 2% FBS in PBST for 1 h at RT in the dark. Cells were then washed 2× with PBST and resuspended in PBS for analysis on the Attune NxT Flow Cytometer (Thermo Fisher Scientific). Data was plotted and analyzed with FlowJo_v10.7.1.

Cell cycle analysis was performed on day 7 after CRISPR/Cas9-mediated knockout of FANCM. Cells (up to 500K) were collected and fixed in 70% ice-cold EtOH. After a wash with PBS, cells were resuspended in FxCycle PI/RNAse Staining Solution (Thermo Fisher #F10797) and incubated for 15 min at RT in the dark. At least 50K events per sample were acquired on the Attune NxT Flow Cytometer and analyzed with FlowJo_v10.7.1.

### 2D Colony Formation Assay:

Cells were washed with PBS and trypsinized with TrypLE until loss of adherence. Cells were collected in a 15 mL falcon tube and spun down into a pellet at 1,000 RPM×5 minutes. The supernatant was aspirated and the cells were resuspended by 5 mL of their appropriate media and counted using Luna cell counter. Cells were diluted to 1-2000 cells/well and plated in triplicate in 6-well plates. The length of the assays varied depending on the lines rate of growth, typically 17-21 days, and on the final day the plates were aspirated, washed and stained with PBS containing 0.5% crystal violet and 3.2% paraformaldehyde. Stained colonies were imaged on the Li-Cor Odyssey CLx (700 nm wavelength).

CRISPR RNP and shRNA:
sgRNA Sequences:

FANCM sgRNA #3:
(SEQ ID NO: 69)
CATGACCACGGCGGCAATAA
FANCM sgRNA #4:
(SEQ ID NO: 70)
AAAGACCTTTATTGCCGCCG
FANCM Avana #1:
(SEQ ID NO: 71)
CCTTTCCTGAAGGGAACCAG
HPRT sgRNA:
(SEQ ID NO: 72)
ATTATGCTGAGGATTTGGAA
PSMA4 sgRNA:
(SEQ ID NO: 73)
AGTCTCGAAGATATGACTCC
PCNA sgRNA:
(SEQ ID NO: 74)
CGAAGATAACGCGGATACCT
Non-targeting sgRNA:
(SEQ ID NO: 75)
GTACGTCGGTATAACTCCTC

sgRNA were provided by Synthego (CRISPRevolution sgRNA EZ Kit—modified) as 10 nmol and resuspended to a 120 uM working solution using nuclease free H20.

Truecut Cas9 V2 (Thermo Fisher Cat #A36498) was used for all CRISPR RNP reactions. 1×10\hat{ }6 cells per RNP reaction were used with 2 ul of Truecut Cas9 VS and 1 uL of sgRNA (120 uM stock). Cas9 and sgRNA's were pre-incubated for 15 minutes before being added to 20 uL solution of cells and then subjected to electroporation using the Lonza Nucelofector 4D system. Electroporated cells were subsequently replated into downstream assays.

shRNA Sequences:

All shRNAs were cloned into pLKO-tet-on vector, packaged into lentivirus and titered for each cell line for an MOI <0.7.

shNT:
(SEQ ID NO: 76)
CAACAAGATGAAGAGCACCAA
shRNA #1307:
(SEQ ID NO: 77)
GACTTCATGAAACTCTATAAT
shRNA #1858:
(SEQ ID NO: 78)
AGGACGAGAGGAACGTATTTA

### Western Blots:

Cells were plated for all conditions at a necessary density for analysis (1×10\hat{ }7 cells per/reaction for nuclear isolation). Following treatment, cells were washed with PBS and trypsinized with TrypLE until loss of adherence. Cells were collected in a 15 mL falcon tube and spun down into a pellet at 1,000 RPM×5 minutes. The supernatant is aspirated and pellets were then placed into downstream protocol (NE-PER cell fractionation kit protocol as provided by vendor). Samples were run on 3-8% Bis-tris gels (Thermo fisher Cat #EA0375PK2) for 45 minutes @ 200 v. Samples were transferred to PVDF membranes using iBlot2 system (IB21001) and subsequently blocked with li-cor blocking buffer (Intercept® (TBS) Blocking Buffer cat #927-60001) for 1 hr at room temp. Membranes were stained overnight at 4 degrees with appropriate antibodies.

### Materials:

### Cell Lines:

UWB1.289 (ovarian; BRCA1 −/− 2475delC)

### UWB1.289+BRCA1

MDA-MB-436 (breast; BRCA1 −/− 5277+1G>A)
COV362 (ovarian; BRCA1 mutant)
TOV21G (ovarian; BRCA1 wild-type)

### Antibodies:

FANCM: Anti-FANCM Antibody, clone CV5.1 (MABC545) 1:1000 dilution

Lamin B: Recombinant Anti-Lamin B1 antibody [EPR8985(B)] (ab133741) 1:2000 dilution

What is claimed is:

1. A composition comprising a Fanconi Anemia Group M protein (FANCM) blocking agent, wherein in a target cell comprising an impaired, defective or deregulated DNA repair pathway, the blocking agent reduces or inhibits a function of FANCM.

2. The composition of claim 1, wherein the target cell is a proliferating cell.

3. The composition of claim 1 or claim 2, wherein the target cell is a tumor cell.

4. The composition of any one of claims 1-3, wherein the target cell is a malignant cell.

5. The composition of any one of claims 1-4, wherein the target cell is a metastatic cell.

6. The composition of any one of claims 1-5, wherein the impaired, defective or deregulated DNA repair pathway is an impaired, defective or deregulated homologous recombination (HR) repair pathway, optionally wherein the target cell comprises a variant protein of the HR pathway.

7. The composition of claim 6, wherein the target cell comprises a BRCA gene deletion, a variant BRCA protein, or a sequence encoding a variant BRCA protein, wherein the variant BRCA protein induces a loss or reduction in a function of the HR pathway.

8. The composition of claim 7, wherein the BRCA gene deletion is a BRCA1 gene deletion, and the variant BRCA protein comprises a variant BRCA1 protein, or wherein the sequence encoding the variant BRCA protein comprises a sequence encoding a variant BRCA1 protein.

9. The composition of claim 7, wherein the BRCA gene deletion is a BRCA2 gene deletion, and the variant BRCA protein comprises a variant BRCA2 protein, or wherein the sequence encoding the variant BRCA protein comprises a sequence encoding a variant BRCA2 protein.

10. The composition of claim 6, wherein the target cell comprises a nucleic acid or an amino acid encoding a variant DNA repair protein RAD51 homolog 1 (RAD51) or a variant homolog of RAD51 and wherein the variant RAD51 induces a loss or reduction in a function of the HR pathway.

11. The composition of claim 6, wherein the target cell comprises a nucleic acid or an amino acid encoding a variant DNA repair protein RAD51 homolog 3 (RAD51C) or a variant homolog of RAD51C and wherein the variant RAD51C induces a loss or reduction in a function of the HR pathway.

12. The composition of claim 6, wherein the target cell comprises a nucleic acid or an amino acid encoding a variant DNA repair protein RAD51 homolog 4 (RAD51D) or a variant homolog of RAD51D and wherein the variant RAD51D induces a loss or reduction in a function of the HR pathway.

13. The composition of claim 6, wherein the target cell comprises a nucleic acid or an amino acid encoding a variant X-ray repair cross-complementing 2 (XRCC2) or a variant homolog of XRCC2 and wherein the variant XRCC2 induces a loss or reduction in a function of the HR pathway.

14. The composition of claim 6, wherein the target cell comprises a nucleic acid or an amino acid encoding a variant DNA repair endonuclease XPF or a variant homolog of XPF and wherein the variant XPF induces a loss or reduction in a function of the HR pathway.

15. The composition of claim 6, wherein the target cell comprises a nucleic acid or an amino acid encoding a variant Meiotic recombination 11 homolog 1 (MRE11A) or a variant homolog of MRE11A and wherein the variant MRE11A induces a loss or reduction in a function of the HR pathway.

16. The composition of claim 6, wherein the target cell comprises a nucleic acid or an amino acid encoding a variant Ataxia telangiectasia mutated (ATM) or a variant homolog of ATM and wherein the variant ATM induces a loss or reduction in a function of the HR pathway.

17. The composition of claim 6, wherein the target cell comprises a nucleic acid or an amino acid encoding a variant BRCA1-associated RING domain protein 1 (BARD1) or a variant homolog of BARD1 and wherein the variant BARD1 induces a loss or reduction in a function of the HR pathway.

18. The composition of claim 6, wherein the target cell comprises a nucleic acid or an amino acid encoding a variant BRCA1-interacting protein C-terminal helicase 1 (BRIP1) or a variant homolog of BRIP1 and wherein the variant BRIP1 induces a loss or reduction in a function of the HR pathway.

19. The composition of claim 6, wherein the target cell comprises a nucleic acid or an amino acid encoding a variant Cell cycle checkpoint kinase (CHEK1) or a variant homolog of CHEK1 and wherein the variant CHEK1 induces a loss or reduction in a function of the HR pathway.

20. The composition of claim 6, wherein the target cell comprises a nucleic acid or an amino acid encoding a variant CHK1 checkpoint homolog (CHEK2) or a variant homolog of CHEK2 and wherein the variant CHEK2 induces a loss or reduction in a function of the HR pathway.

21. The composition of claim 6, wherein the target cell comprises a nucleic acid or an amino acid encoding a variant Nibrin (NBN) or a variant homolog of NBN and wherein the variant NBN induces a loss or reduction in a function of the HR pathway.

22. The composition of claim 6, wherein the target cell comprises a nucleic acid or an amino acid encoding a variant Partner and localizer of BRCA2 (PALB2) or a variant homolog of PALB2 and wherein the variant PALB2 induces a loss or reduction in a function of the HR pathway.

23. The composition of claim 6, wherein the target cell comprises a nucleic acid or an amino acid encoding a variant Structure-specific endonuclease subunit SLX4 (SLX4) or a variant homolog of SLX4 and wherein the variant SLX4 induces a loss or reduction in a function of the HR pathway.

24. The composition of any one of claims 7-23, wherein the variant protein or the sequence encoding the variant protein comprises one or more of a mutation, a deletion, a promotor methylation, a silencing event and a splicing event.

25. The composition of claim 24, wherein the mutation comprises one or more of a substitution, an insertion, a deletion, an inversion, and a translocation of a nucleic acid sequence or an amino acid sequence encoding the variant BRCA protein, optionally wherein the mutation is BRCA1 or BRCA2 gene deletion, or wherein the variant BRCA protein is a variant BRCA1 protein or a variant BRCA2 protein.

26. The composition of claim 24 or 25, wherein the mutation introduces a stop codon into a nucleic acid sequence encoding the variant protein, thereby generating one or more of a truncated protein, an inactivated protein and a protein fragment.

27. The composition of any one of claims 3-26, wherein the variant protein or the sequence encoding the variant protein comprises a promoter sequence, and wherein the promoter controls expression of the variant protein or the sequence encoding the variant protein.

28. The composition of claim 27, wherein the silencing event comprises a silencing of the promoter sequence, function, or activity.

29. The composition of claim 27 or 28, wherein the promoter sequence controlling expression of the variant protein or the sequence encoding the variant protein comprises a mutation.

30. The composition of claim 29, wherein the mutation comprises one or more of a substitution, an insertion, a deletion, an inversion, and a translocation of the promoter sequence.

31. The composition of any one of claims 1-30, wherein the function of FANCM comprises one or more of ATP-binding, nucleotide-binding, DNA-binding, DNA remodeling, DNA strand separation, DNA-RNA strand separation and catalyzing the break of a chemical bond using water.

32. The composition of any one of claims 1-30, wherein the function of FANCM comprises a helicase activity.

33. The composition of any one of claims 1-30, wherein the function of FANCM comprises a hydrolase activity.

34. The composition of any one of claims 1-30, wherein the function of FANCM comprises a translocase activity.

35. The composition of any one of claims 1-34, wherein the function of FANCM comprises an ATPase activity.

36. The composition of any one of claims 6-35, wherein the variant protein increases a function of FANCM.

37. The composition of any one of claims 6-35, wherein the variant protein decreases a function of FANCM.

38. The composition of any one of claims 1-30, wherein the impairment, defect or deregulation of the HR pathway increases a dependence of the target cell upon a function of FANCM.

39. The composition of any one of claims 6-38, wherein the function of the HR pathway comprises one or more of
i) recognizing nucleotide or DNA damage;
ii) recruiting a protein to a site of nucleotide or DNA damage;
iii) configuring or remodeling a sequence comprising a site of nucleotide or DNA damage;
iv) configuring or remodeling a sequence complementary to a site of nucleotide or DNA damage;
v) inducing a break in a sequence within a site of nucleotide or DNA damage;
vi) inducing a break in a sequence comprising the site of nucleotide or DNA damage;
vii) inducing a break in a sequence complementary to a site of nucleotide or DNA damage;
viii) removing a sequence within a site of nucleotide or DNA damage;
ix) removing a sequence comprising a site of nucleotide or DNA damage;
x) synthesizing a new sequence within a site of nucleotide or DNA damage;
xi) synthesizing a new sequence comprising a site of nucleotide or DNA damage;
xii) resecting a portion of a synthesized sequence within a site of nucleotide or DNA damage;
xiii) resecting a portion of a synthesized sequence comprising the site of nucleotide or DNA damage;
xiv) stabilizing a site of DNA synthesis or replication within a site of nucleotide or DNA damage;
xv) stabilizing a site of DNA synthesis or replication comprising a site of nucleotide or DNA damage;
xvi) stabilizing a site of DNA synthesis or replication comprising a target site;
xvii) stabilizing a site of DNA synthesis or replication comprising a stalled replication fork;
xviii) inducing or facilitating invasion of a synthesized sequence within the site of nucleotide or DNA damage;
xix) inducing or facilitating invasion of a synthesized sequence comprising the site of nucleotide or DNA damage;
xx) inducing or facilitating insertion of a synthesized sequence within the site of nucleotide or DNA damage by recombination; and
xxi) inducing or facilitating insertion of a synthesized sequence comprising the site of nucleotide or DNA damage by recombination.

40. The composition of any one of claims 6-39, wherein an activity of the HR pathway comprises an increase or a decrease in a function of a component of the HR pathway.

41. The composition of any one of claims 6-40, wherein the variant protein increases a function of a component of the HR pathway.

42. The composition of any one of claims 6-40, wherein the variant protein decreases a function of a component of the HR pathway.

43. The composition of any one of claims 1-42, further comprising a pharmaceutically-acceptable carrier.

44. The composition of any one of claims 1-43, wherein the blocking agent comprises an effector moiety that binds to a FANCM protein or a nucleic acid sequence encoding the FANCM protein.

45. The composition of claim 44, wherein the effector moiety comprises one or more of an ion, a small molecule, a single-stranded nucleic acid molecule, a double-stranded nucleic acid molecule, an aptamer, an RNA-guided nuclease, a DNA-guided nuclease, a polypeptide, an antibody, a functional fragment of an antibody, an antibody mimetic, a scaffold, a matrix, or any combination thereof.

46. The composition of any one of claims 1-45, wherein the blocking agent further comprises a targeting moiety operably-linked to the effector moiety.

47. The composition of claim 46, wherein the targeting moiety is reversibly-linked to the effector moiety.

48. The composition of claim 46 or 47, wherein the targeting moiety specifically binds a component of the target cell.

49. The composition of any one of claims 1-48, wherein the target cell is a proliferating cell.

50. The composition of any one of claims 1-49, wherein the target cell is a tumor cell.

51. The composition of any one of claims 1-50, wherein the target cell is a malignant cell.

52. The composition of any one of claims 1-51, wherein the target cell is a metastatic cell.

53. The composition of any one of claims 1-52, wherein the target cell is produced or derived from a non-hematological tissue.

54. The composition of any one of claims 1-53, wherein the target cell is produced or derived from an epithelial tissue.

55. The composition of any one of claims 1-54, wherein the target cell is produced or derived from an organ or a structure comprising an epithelial tissue.

56. The composition of any one of claims 1-55, wherein the target cell is produced or derived from a skin area, a skin layer, a lung, a lymph node, a breast, an ovary, a prostate, a mouth, a nose, a nasal passage, an esophagus, an intestine, a small intestine, a large intestine, a stomach, a kidney, a liver, a spleen, a heart, an artery, a vein, a bladder and a colon.

57. The composition of any one of claims 1-52, wherein the target cell is produced or derived from a bone or a connective tissue.

58. The composition of any one of claims 1-57, wherein the blocking agent further comprises a regulation moiety.

59. The composition of claim 58, wherein the regulation moiety is operably-linked to one or more of the effector moiety and the targeting moiety.

60. The composition of claim 58 or 59, wherein the regulation moiety is reversibly-linked to one or more of the effector moiety and the targeting moiety.

61. The composition of any one of claims 58-60, wherein the regulation moiety selectively binds a component not present in a target cell.

62. The composition of claim 61, wherein the component not present in a target cell is present in a healthy cell.

63. The composition of claim 61 or 62, wherein the component decreases or inhibits an activity of the effector moiety.

64. The composition of any one of claims 58-63, wherein the regulation moiety comprises a microRNA (miRNA) binding site and selectively binds a miRNA.

65. The composition of any one of claims 1-64, wherein the target cell is an ALT+ cell.

66. The composition of any one of claims 1-64, wherein the target cell is not an ALT+ cell.

67. A method of inducing cell death in a proliferating cell, comprising contacting the proliferating cell with the composition of any one of claims 1-66.

68. The method of claim 67, wherein the cell is in vitro or ex vivo.

69. The method of claim 67, wherein the cell is in vivo.

70. A method of inducing cell cycle arrest in a proliferating cell, comprising contacting the proliferating cell with the composition of any one of claims 1-66.

71. The method of claim 70, wherein the proliferating cell is in vitro or ex vivo.

72. The method of claim 71, wherein the proliferating cell is in vivo.

73. The method of any one of claims 67-72, wherein the proliferating cell comprises the impaired, defective or deregulated DNA repair pathway.

74. The method of claim 73, wherein the proliferating cell comprises the impaired, defective or deregulated homologous recombination (HR) repair pathway.

75. The method of claim 74, wherein the proliferating cell comprises a variant BRCA protein or a sequence encoding a variant BRCA protein, and wherein the variant BRCA protein induces a loss or reduction in a function of the HR pathway.

76. The method of claim 75, wherein the variant BRCA protein comprises a variant BRCA1 protein or a variant BRCA2 protein, or wherein the sequence encoding the variant BRCA protein comprises a sequence encoding a variant BRCA1 protein or a variant BRCA2 protein.

77. The method of any one of claims 70-76, wherein the proliferating cell is an ALT+ cell.

78. The method of any one of claims 70-76, wherein the proliferating cell is not an ALT+ cell.

79. The method of any one of claims 70-78, wherein the proliferating cell is resistant to a PARP inhibitor.

80. The method of any one of claims 70-79, wherein the proliferating cell is an ovarian tumor cell or a breast tumor cell, optionally a BRCA1^{-/-} tumor cell or a tumor cell comprising the variant BRCA1 protein, or a sequence encoding the variant BRCA protein.

81. A method of treating cancer, comprising administrating to a subject an effective amount of the composition of any one of claims 1-66.

82. The method of claim 81, wherein the method further comprises administering a second therapy.

83. The method of claim 82, wherein the second therapy comprises radiation and/or a chemotherapy.

84. The method of claim 83, wherein the chemotherapy comprises a Poly (ADP-ribose) polymerase (PARP) inhibitor or a platinum-based therapy.

85. The method of any one of claims 81-84, wherein the cancer is resistant to treatment with a PARP inhibitor as a monotherapy.

86. The method of any one of claims 81-85, wherein, prior to administration of the composition, the subject has been identified as resistant to treatment with a PARP inhibitor as a monotherapy.

87. The method of any one of claims 81-86, wherein, prior to administration of the composition, the subject has been treated with a PARP inhibitor as a monotherapy.

88. The method of any one of claims 81-87, wherein the administration is systemic.

89. The method of claim 88, wherein the composition is administered by one or more of an oral route, an inhaled route, an intravenous route, an intraperitoneal route, and a subcutaneous route.

90. The method of any one of claims 81-87, wherein the administration is local.

91. The method of claim 90, wherein the composition is administered by one or more of an intraocular route, an intraspinal route, an intracerebellar route, an intrathecal route, an intramuscular route and an intraosseous route.

92. The method of any one of claims 81-91, wherein the composition is administered once per day, twice per day or three times per day.

93. The method of any one of claims 81-92, wherein the composition is administered once per week, twice per week or three times per week.

94. The method of any one of claims 81-93, wherein the composition is administered once per month, twice per month or three times per month.

95. The method of any one of claims 81-94, wherein treating comprises a reduction in a severity of a sign or symptom of the cancer.

96. The method of any one of claims 81-95, wherein treating comprises a reduction in a volume of a tumor.

97. The method of any one of claims 81-96, wherein treating comprises a reduction in a number of tumor cells per volume of blood or mass of tissue.

98. The method of any one of claims 81-97, wherein treating comprises a remission.

99. The method of any one of claims 81-98, wherein treating comprises an increased duration of progression free survival.

100. The method of any one of claims 81-99, wherein the cancer comprises cancer cells comprising an impaired, defective or deregulated DNA repair pathway.

101. The method of claim 100, wherein the cancer cells comprise an impaired, defective or deregulated homologous recombination (HR) repair pathway.

102. The method of claim 101, wherein the cancer cells comprise a variant BRCA protein or a sequence encoding a variant BRCA protein, and wherein the variant BRCA protein induces a loss or reduction in a function of the HR pathway.

103. The method of claim 102, wherein the variant BRCA protein comprises a variant BRCA1 protein or a variant BRCA2 protein, or wherein the sequence encoding the variant BRCA protein comprises a sequence encoding a variant BRCA1 protein or a variant BRCA2 protein.

104. The method of any one of claims 81-103, wherein the cancer cells are ALT+ cells.

105. The method of any one of claims 81-103, wherein the cancer cells are not an ALT+ cells.

106. The method of any one of claims 81-105, wherein the cancer cells are resistant to a PARP inhibitor.

107. The method of any one of claims 81-106, wherein the cancer is an ovarian cancer or a breast cancer, optionally a BRCA1^{-/-} cancer or a cancer comprising tumor cells comprising a variant BRCA1 protein, or a sequence encoding the variant BRCA protein.

108. The method of claim 81, wherein the method comprises administering to a subject having a BRCA1−/− ovarian cancer or BRCA1−/− breast cancer a Fanconi Anemia Group M protein (FANCM) blocking agent.

109. The method of claim 108, wherein the method results in DNA damage and/or cell cycle arrest of BRCA1−/− ovarian cancer cells or BRCA1−/− breast cancer cells.

110. The method of claim 108 or claim 109, wherein the method decreases survival or growth of BRCA1−/− ovarian cancer cells or BRCA1−/− breast cancer cells.


SEQUENCE LISTING (78 sequences)

SEQ ID NO: 1 - protein, 919 aa, Homo sapiens
MQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 2 - DNA, 3410 bp, Homo sapiens
ctcgcgggggcgcttccaccgattcctcctctttccctgccagtcactcctcagaccctcagccacacccgctcatccagggcgagggaaagcgcgggcattttcccagtgtgctctgcgggagggctcgccccacttcaccccttttcccgccctcctcccattcgggagactacgactcccagtgtcctccgcgcgacggcggcggtgcggacggtgcccaggtcccgcccctaggctctgccccgcccccgcccgcagacgtctgcgcgcgaatgccgtggcgcgaacttgggactgcagaggcgcgcctggcggatctgagtgtgttgcccgggcagcggcgcgcgggaccaacgcaaggagcagctgacagacgaagaaaagtgctggacaggaagggagaattctgacgccaacatgcagcgaagtatcatgtcatttttccaccccaagaaagagggtaaagcaaagaagcctgagaaggaggcatccaatagcagcagagagacggagccccctccaaaggcggcactgaaggagtggaatggagtggtgtccgagagtgactctccggtgaagaggccagggaggaaggcggcccgggtcctgggcagcgaaggggaagaggaggatgaagcccttagccctgctaaaggccagaagcctgccctggactgctcacaggtctccccgccccgtcctgccacatctcctgagaacaatgcttccctctctgacacctctcccatggacagttccccatcagggattccgaagcgtcgcacagctcggaagcagctcccgaaacggaccattcaggaagtcctggaagagcagagtgaggacgaggacagagaagccaagaggaagaaggaggaggaagaagaggagaccccgaaagaaagcctcacagaggctgaagtggccacagagaaggaaggagaagacggggaccagcccaccacgcctcccaagcccctaaagacctccaaagcagagaccccgacggaaagcgtttcagagcctgaggtggccacgaagcaggaactgcaggaggaggaagagcagaccaagcctccccgcagagctcccaagacgctcagcagcttcttcaccccccggaagccagcagtcaaaaaagaagtgaaggaagaggagccaggggctccaggaaaggagggagctgctgagggacccctggatccatctggttacaatcctgccaagaacaactatcatcccgtggaagatgcctgctggaaaccgggccagaaggttccttacctggctgtggcccggacgtttgagaagatcgaggaggtgtctgctcggctccggatggtggagacgctgagcaacttgctgcgctccgtggtggccctgtcgcctccagacctcctccctgtcctctacctcagcctcaaccaccttgggccaccccagcagggcctggagcttggcgtgggtgatggtgtccttctcaaggcagtggcccaggccacaggtcggcagctggagtccgtccgggctgaggcagccgagaaaggcgacgtggggctggtggccgagaacagccgcagcacccagaggctcatgctgccaccacctccgctcactgcctccggggtcttcagcaagttccgcgacatcgccaggctcactggcagtgcttccacagccaagaagatagacatcatcaaaggcctctttgtggcctgccgccactcagaagcccggttcatcgctaggtccctgagcggacggctgcgccttgggctggcagagcagtcggtgctggctgccctctcccaggcagtgagcctcacgcccccgggccaagaattcccaccagccatggtggatgctgggaagggcaagacagcagaggccagaaagacgtggctggaggagcaaggcatgatcctgaagcagacgttctgcgaggttcccgacctggaccgaattatccccgtgctgctggagcacggcctggaacgtctcccggagcactgcaagctgagcccagggattcccctgaaaccaatgttggcccatcccacccggggcatcagcgaggtcctgaaacgctttgaggaggcagctttcacctgcgaatacaaatatgacgggcagagggcacagatccacgccctggaaggcggggaggtgaagatcttcagcaggaatcaggaagacaacactgggaagtacccggacatcatcagccgcatccccaagattaaactcccatcggtcacatccttcatcctggacaccgaagccgtggcttgggaccgggaaaagaagcagatccagccattccaagtgctcaccacccgcaaacgcaaggaggtggatgcgtctgagatccaggtgcaggtgtgtttgtacgccttcgacctcatctacctcaatggagagtccctggtacgtgagcccctttcccggcgccggcagctgctccgggagaactttgtggagacagagggcgagtttgtcttcgccacctccctggacaccaaggacatcgagcagatcgccgagttcctggagcagtcagtgaaagactcctgcgaggggctgatggtgaagaccctggatgttgatgccacctacgagatcgccaagagatcgcacaactggctcaagctgaagaaggactaccttgatggcgtgggtgacaccctggacctggtggtgatcggcgcctacctgggccgggggaagcgggccggccggtacgggggcttcctgctggcctcctacgacgaggacagtgaggagctgcaggccatatgcaagcttggaactggcttcagtgatgaggagctggaggagcatcaccagagcctcaaggcgctggtgctgcccagcccacgcccttacgtgcggatagatggcgctgtgattcccgaccactggctggaccccagcgctgtgtgggaggtgaagtgcgctgacctctccctctctcccatctaccctgctgcgcggggcctggtggatagtgacaagggcatctcccttcgcttccctcggtttattcgagtccgtgaagacaagcagccggagcaggccaccaccagtgctcaggtggcctgtttgtaccggaagcaaagtcagattcagaaccaacaaggcgaggactcaggctctgaccctgaagatacctactaagccctcgccctcctagggcctgggtacagggcatgagttggacggaccccagggttattattgcctttgctttttagcaaatctgctgtggcaggctgtggattttgagagtcaggggaggggtgtgtgtgtgagggggtggcttactccggagtctgggattcatcccgtcatttctttcaataaataattattggatagctaaaaaaaaaaaaaaaaaaaaaaaaaaa

SEQ ID NO: 3 - protein, 801 aa, Homo sapiens
MQRSIMSFFHPKKEGKAKKPEKEASNSSRETEPPPKAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKVLGNWG

SEQ ID NO: 4 - DNA, 3957 bp, Homo sapiens
gaccaacgcaaggagcagctgacagacgaagaaaagtgctggacaggaagggagaattctgatgccaacatgcagcgaagtatcatgtcatttttccaccccaagaaagagggtaaagcaaagaagcctgagaaggaggcatccaatagcagcagagagacggagccccctccaaaggcggcactgaaggagtggaatggagtggtgtccgagagtgactctccggtgaagaggccagggaggaaggcggcccgggtcctgggcagcgaaggggaagaggaggatgaagcccttagccctgctaaaggccagaagcctgccctggactgctcacaggtctccccgccccgtcctgccacatctcctgagaacaatgcttccctctctgacacctctcccatggacagttccccatcagggattccgaagcgtcgcacagctcggaagcagctcccgaaacggaccattcaggaagtcctggaagagcagagtgaggacgaggacagagaagccaagaggaagaaggaggaggaagaagaggagaccccgaaagaaagcctcacagaggctgaagtggcaacagagaaggaaggagaagacggggaccagcccaccacgcctcccaagcccctaaagacctccaaagcagagaccccgacggaaagcgtttcagagcctgaggtggccacgaagcaggaactgcaggaggaggaagagcagaccaagcctccccgcagagctcccaagacgctcagcagcttcttcaccccccggaagccagcagtcaaaaaagaagtgaaggaagaggagccaggggctccaggaaaggagggagctgctgagggacccctggatccatctggttacaatcctgccaagaacaactatcatcccgtggaagatgcctgctggaaaccgggccagaaggttccttacctggctgtggcccggacgtttgagaagatcgaggaggtgtctgctcggctccggatggtggagacgctgagcaacttgctgcgctccgtggtggccctgtcgcctccagacctcctccctgtcctctacctcagcctcaaccaccttgggccaccccagcagggcctggagcttggcgtgggtgatggtgtccttctcaaggcagtggcccaggccacaggtcggcagctggagtccgtccgggctgaggcagccgagaaaggcgacgtggggctggtggccgagaacagccgcagcacccagaggctcatgctgccaccacctccgctcactgcctccggggtcttcagcaagttccgcgacatcgccaggctcactggcagtgcttccacagccaagaagatagacatcatcaaaggcctctttgtggcctgccgccactcagaagcccggttcatcgctaggtccctgagcggacggctgcgccttgggctggcagagcagtcggtgctggctgccctctcccaggcagtgagcctcacgcccccgggccaagaattcccaccagccatggtggatgctgggaagggcaagacagcagaggccagaaagacgtggctggaggagcaaggcatgatcctgaagcagacgttctgcgaggttcccgacctggaccgaattatccccgtgctgctggagcacggcctggaacgtctcccggagcactgcaagctgagcccagggattcccctgaaaccaatgttggcccatcccacccggggcatcagcgaggtcctgaaacgctttgaggaggcagctttcacctgcgaatacaaatatgacgggcagagggcacagatccacgccctggaaggcggggaggtgaagatcttcagcaggaatcaggaagacaacactgggaagtacccggacatcatcagccgcatccccaagattaaactcccatcggtcacatccttcatcctggacaccgaagccgtggcttgggaccgggaaaagaagcagatccagccattccaagtgctcaccacccgcaaacgcaaggaggtggatgcgtctgagatccaggtgcaggtgtgtttgtacgccttcgacctcatctacctcaatggagagtccctggtacgtgagcccctttcccggcgccggcagctgctccgggagaactttgtggagacagagggcgagtttgtcttcgccacctccctggacaccaaggacatcgagcagatcgccgagttcctggagcagtcagtgaaagactcctgcgaggggctgatggtgaagaccctggatgttgatgccacctacgagatcgccaagagatcgcacaactggctcaagctgaagaaggactaccttgatggcgtgggtgacaccctggacctggtggtgatcggcgcctacctgggccgggggaagcgggccggccggtacgggggcttcctgctggcctcctacgacgaggacagtgaggagctgcaggccatatgcaaggtcctggggaactggggctgatggcagaagcaggagggaaggagattagactcccgggggtatttcctattgcacaacccagaccgaggggtgctcacatccccctgtgaaggatccgttctccccagtttttatttactttttctggttctatgagcaactcttgcttcatatagaaaaacaggctgggatagaaagatgtcaggaatgtgtgtcagtgagagattgagattagctacaaatagtggtggctcacataagaaaagaatttttttgttatgaaatgagaagtgtggagggggcagtctgatgccattagggattcaggttcctcctctccccacatttttttttgtccccacattttttttttctgccatgtgcctctggcctcatcatcacaatgtggctgcaggagttccagccctcatggccatgcctgctccacgcaccagggaagagcaaagggggttggaccctgagtcagcctcagccagtgaacaccttctgctcaagttctgttgctcatcactttgctccatggctcacccctagctgcaaggaattctgggaaatgtaaagtgatttactttttttcaagttaaagtttttcttaaagctgtgcacattttggactagattttcttacgtatttcctatattcccctctccaacaaatttgggtcttttgccacagaaacagaacaaaatatcacctagaatcgtaccacccagagacacgcagtagtgacatttgagggaatgttttatttacatttttgcaaagttgtgatccttctctatgagtaattttcttttctgcctttttaccctggaagtactttgttttcagtgttgtatatgcccttaagcaatatggcaacaggacctgaatgcccgcacaagagttttcaagtgctgtgccccatagcacatagtgctgtgtcctgaaccagctccccgtggctgtgtggggaagttgttgccggctttgttctgatggcttggaactggcttcagtgacgaggagctggaggagcatcaccagagcctcaaggcgctggtgctgcccagcccacgcccttacgtgcggatagatggcgctgtgattcccgaccactggctggaccccagcgctgtgtgggaggtgaagtgcgctgacctctccctctctcccatctaccctgctgcgcggggcctggtggatagtgacaagggcatctcccttcgcttccctcggtttattcgagtccgtgaagacaagcagccggagcaggccaccaccagtgctcaggtggcctgtttgtaccggaagcaaagtcagattcagaaccaacaaggcgaggactcaggctctgaccctgaagatacctactaagccctcgccctcctagggcctgggtacagggcatgagttggacggaccccagggttattattgcctttgctttttagcaaatctgctgtggcaggctgtggattttgagagtcaggggaggggtg

SEQ ID NO: 5 - protein, 888 aa, Homo sapiens
MQRSIMAALKEWNGVVSESDSPVKRPGRKAARVLGSEGEEEDEALSPAKGQKPALDCSQVSPPRPATSPENNASLSDTSPMDSSPSGIPKRRTARKQLPKRTIQEVLEEQSEDEDREAKRKKEEEEETPKESLTEAEVATEKEGEDGDQPTTPPKPLKTSKAETPTESVSEPEVATKQELQEEEEQTKPPRRAPKTLSSFFTPRKPAVKKEVKEEEPGAPGKEGAAEGPLDPSGYNPAKNNYHPVEDACWKPGQKVPYLAVARTFEKIEEVSARLRMVETLSNLLRSVVALSPPDLLPVLYLSLNHLGPPQQGLELGVGDGVLLKAVAQATGRQLESVRAEAAEKGDVGLVAENSRSTQRLMLPPPPLTASGVFSKFRDIARLTGSASTAKKIDIIKGLFVACRHSEARFIARSLSGRLRLGLAEQSVLAALSQAVSLTPPGQEFPPAMVDAGKGKTAEARKTWLEEQGMILKQTFCEVPDLDRIIPVLLEHGLERLPEHCKLSPGIPLKPMLAHPTRGISEVLKRFEEAAFTCEYKYDGQRAQIHALEGGEVKIFSRNQEDNTGKYPDIISRIPKIKLPSVTSFILDTEAVAWDREKKQIQPFQVLTTRKRKEVDASEIQVQVCLYAFDLIYLNGESLVREPLSRRRQLLRENFVETEGEFVFATSLDTKDIEQIAEFLEQSVKDSCEGLMVKTLDVDATYEIAKRSHNWLKLKKDYLDGVGDTLDLVVIGAYLGRGKRAGRYGGFLLASYDEDSEELQAICKLGTGFSDEELEEHHQSLKALVLPSPRPYVRIDGAVIPDHWLDPSAVWEVKCADLSLSPIYPAARGLVDSDKGISLRFPRFIRVREDKQPEQATTSAQVACLYRKQSQIQNQQGEDSGSDPEDTY

SEQ ID NO: 6 - DNA, 3317 bp, Homo sapiens
ctcgcgggggcgcttccaccgattcctcctctttccctgccagtcactcctcagaccctcagccacacccgctcatccagggcgagggaaagcgcgggcattttcccagtgtgctctgcgggagggctcgccccacttcaccccttttcccgccctcctcccattcgggagactacgactcccagtgtcctccgcgcgacggcggcggtgcggacggtgcccaggtcccgcccctaggctctgccccgcccccgcccgcagacgtctgcgcgcgaatgccgtggcgcgaacttgggactgcagaggcgcgcctggcggatctgagtgtgttgcccgggcagcggcgcgcgggaccaacgcaaggagcagctgacagacgaagaaaagtgctggacaggaagggagaattctgacgccaacatgcagcgaagtatcatggcggcactgaaggagtggaatggagtggtgtccgagagtgactctccggtgaagaggccagggaggaaggcggcccgggtcctgggcagcgaaggggaagaggaggatgaagcccttagccctgctaaaggccagaagcctgccctggactgctcacaggtctccccgccccgtcctgccacatctcctgagaacaatgcttccctctctgacacctctcccatggacagttccccatcagggattccgaagcgtcgcacagctcggaagcagctcccgaaacggaccattcaggaagtcctggaagagcagagtgaggacgaggacagagaagccaagaggaagaaggaggaggaagaggagaccccgaaagaaagcctcacagaggctgaagtggccacagagaaggaaggagaagacggggaccagcccaccacgcctcccaagcccctaaagacctccaaagcagagaccccgacggaaagcgtttcagagcctgaggtggccacgaagcaggaactgcaggaggaggaagagcagaccaagcctccccgcagagctcccaagacgctcagcagcttcttcaccccccggaagccagcagtcaaaaaagaagtgaaggaagaggagccaggggctccaggaaaggagggagctgctgagggacccctggatccatctggttacaatcctgccaagaacaactatcatcccgtggaagatgcctgctggaaaccgggccagaaggttccttacctggctgtggcccggacgtttgagaagatcgaggaggtgtctgctcggctccggatggtggagacgctgagcaacttgctgcgctccgtggtggccctgtcgcctccagacctcctccctgtcctctacctcagcctcaaccaccttgggccaccccagcagggcctggagcttggcgtgggtgatggtgtccttctcaaggcagtggcccaggccacaggtcggcagctggagtccgtccgggctgaggcagccgagaaaggcgacgtggggctggtggccgagaacagccgcagcacccagaggctcatgctgccaccacctccgctcactgcctccggggtcttcagcaagttccgcgacatcgccaggctcactggcagtgcttccacagccaagaagatagacatcatcaaaggcctctttgtggcctgccgccactcagaagcccggttcatcgctaggtccctgagcggacggctgcgccttgggctggcagagcagtcggtgctggctgccctctcccaggcagtgagcctcacgcccccgggccaagaattcccaccagccatggtggatgctgggaagggcaagacagcagaggccagaaagacgtggctggaggagcaaggcatgatcctgaagcagacgttctgcgaggttcccgacctggaccgaattatccccgtgctgctggagcacggcctggaacgtctcccggagcactgcaagctgagcccagggattcccctgaaaccaatgttggcccatcccacccggggcatcagcgaggtcctgaaacgctttgaggaggcagctttcacctgcgaatacaaatatgacgggcagagggcacagatccacgccctggaaggcggggaggtgaagatcttcagcaggaatcaggaagacaacactgggaagtacccggacatcatcagccgcatccccaagattaaactcccatcggtcacatccttcatcctggacaccgaagccgtggcttgggaccgggaaaagaagcagatccagccattccaagtgctcaccacccgcaaacgcaaggaggtggatgcgtctgagatccaggtgcaggtgtgtttgtacgccttcgacctcatctacctcaatggagagtccctggtacgtgagcccctttcccggcgccggcagctgctccgggagaactttgtggagacagagggcgagtttgtcttcgccacctccctggacaccaaggacatcgagcagatcgccgagttcctggagcagtcagtgaaagactcctgcgaggggctgatggtgaagaccctggatgttgatgccacctacgagatcgccaagagatcgcacaactggctcaagctgaagaaggactaccttgatggcgtgggtgacaccctggacctggtggtgatcggcgcctacctgggccgggggaagcgggccggccggtacgggggcttcctgctggcctcctacgacgaggacagtgaggagctgcaggccatatgcaagcttggaactggcttcagtgatgaggagctggaggagcatcaccagagcctcaaggcgctggtgctgcccagcccacgcccttacgtgcggatagatggcgctgtgattcccgaccactggctggaccccagcgctgtgtgggaggtgaagtgcgctgacctctccctctctcccatctaccctgctgcgcggggcctggtggatagtgacaagggcatctcccttcgcttccctcggtttattcgagtccgtgaagacaagcagccggagcaggccaccaccagtgctcaggtggcctgtttgtaccggaagcaaagtcagattcagaaccaacaaggcgaggactcaggctctgaccctgaagatacctactaagccctcgccctcctagggcctgggtacagggcatgagttggacggaccccagggttattattgcctttgctttttagcaaatctgctgtggcaggctgtggattttgagagtcaggggaggggtgtgtgtgtgagggggtggcttactccggagtctgggattcatcccgtcatttctttcaataaataattattggatagctaaaaaaaaaaaaaaaaaaaaaaaaaaa

SEQ ID NO: 7 - protein, 2048 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 8 - DNA, 7138 bp, Homo sapiens
tgtgcgaaggaaaccgatggggatcggaaccgtagcggttgagctgctgctgctacggatatctgacagaagccttcggtggttgtcggcctaatgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggttgtaagagaactagtcaaatatacaaatcactttagaatcttggctctaagtgccacaccaggtagtgatataaaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagctcaagaatcaaccactggaattcatcaactctctctctctgaatggagactgtggcaagatcatcctttgcctacacatcaagttgatcactcagatcgatgccgccattttataggccttatgcaaatgatagagggaatgagacacgaagagggagaatgcagctatgaattggaagttgaatcttatttacaaatggaagatgttacctcaacatttattgctcccaggaatgaatctaataatcttgccagtgacacctttatcactcacaagaaatcgtcatttataaagaacataaatcaaggcagttcatcctcagtgatagaatctgatgaagaatgtgctgaaattgttaaacaaactcatatcaaacctactaaaattgtttctttaaagaaaaaagtgtctaaagaaataaaaaaagatcagcttaaaaaagaaaataatcacggtattatagattctgtagataatgacagaaattccactgttgaaaatatttttcaagaagacctaccaaatgataaaaggacatcagatacagatgaaattgctgccacatgtactattaatgaaaatgttattaaagaaccgtgtgtgttattaacagagtgtcagtttacaaataaatccactagttcacttgctggaaatgttttagattctggttataacagtttcaatgatgaaaaatctgtttcatctaacttatttcttccattcgaagaagagctttatattgttagaacagatgaccaattttataattgtcactcattgacaaaagaggtactagctaatgtagagagatttttatcttattctcctccgcctctcagtggactctcagacttggaatatgaaattgctaagggtactgcacttgagaatttgcttttcttaccctgtgcagagcatttacgaagtgataaatgcacctgtttgctgtcacattcagctgtgaattctcaacagaatttagaattgaattcacttaaatgtataaattatccatctgaaaaaagttgcctttatgatatacctaatgataatatttctgatgagccaagtctctgtgactgtgatgtacataaacataatcaaaatgaaaatttagtacctaacaatcgtgttcaaatacacagaagccctgcacagaatttagttggagagaacaatcatgatgttgataacagtgacctcccagtattgtccactgatcaagatgaaagtttgctgttatttgaagatgttaatacagagttcgacgatgtgagtctttcacccttgaacagtaaaagcgaatctttacctgtgtcagacaaaactgctattagtgaaacgcctctggtctctcagttcttaatttctgatgaacttttgttggacaataattctgaactccaagatcaaatcacccgtgatgctaatagttttaaatctcgtgatcagagaggtgtacaggaagaaaaagtgaagaatcatgaggatatttttgattgctctagggatttattttctgttacctttgatttaggattctgtagtccagattctgatgatgaaatattggaacatacatcagatagcaatagacctctagatgatctatatggaaggtatttggaaattaaggagataagtgatgcaaattatgtttcgaatcaagcactaataccaagagatcatagtaaaaattttactagtggaactgttattatcccatcaaatgaagatatgcagaatccaaattatgtacatttgccactgagtgcagcaaaaaatgaagaattgttatctcctggttattctcagttttctttaccagtgcaaaaaaaagttatgagtacaccactctctaaatcaaacacattgaactcattttctaagataagaaaggaaatacttaagacaccagattctagtaaggaaaaagtaaacctacaaagattcaaagaagcattgaattcaacttttgattattcagaattttctctagaaaagtctaaaagcagtggtccaatgtatctgcataaatcctgtcattctgttgaagatggacaattattaacaagtaacgaaagtgaagatgacgagattttccgaagaaaagttaaaagagcaaaaggaaatgttttaaactctcctgaggatcagaaaaatagtgaagttgattctccacttcatgctgtcaaaaagcgcagatttcctataaacagatcagaattatcatctagtgatgagagtgagaattttcccaaaccatgttcacaattagaagacttcaaggtttgtaacgggaatgccagaagaggcatcaaagtcccaaagagacagagtcacttaaagcatgtagctaggaagtttttagatgatgaagcagaactttctgaagaagatgcagaatatgtttcatcagatgaaaatgatgagtcagaaaatgaacaagattcctcattacttgactttttaaatgatgaaactcaactttcacaggctataaatgattctgaaatgagagctatttacatgaaatctttgcgtagtccaatgatgaacaataagtacaaaatgattcataagacacataaaaacataaacattttctcgcagattcctgaacaagatgaaacctatttagaggatagtttttgtgttgatgaagaggagtcttgcaaaggccaatcaagtgaagaagaagtttgtgttgattttaacttaataactgatgattgctttgcaaatagtaaaaagtataaaactcgacgtgcagtaatgctaaaagaaatgatggaacaaaattgtgcacattcaaaaaagaaattatccagaattattttaccagatgattcaagtgaggaggagaacaatgtaaatgataaaagagaatctaatattgcggttaacccaagcactgttaagaagaacaaacaacaggaccattgtttaaattcagtgccttctggatcttctgcgcagtccaaggtgcgttctactccaagagttaatccattagcaaagcagagcaaacagacatcgctgaatttaaaggatacaatttccgaagtctcagacttcaaacctcagaatcataatgaagtccagtctaccacaccacccttcactactgttgattcacagaaagactgtagaaaatttccagttccacagaaggatggtagtgctttggaggattctagcacttcaggggcatcctgttccaagtcaagaccacatttagctgggacacatacttctcttagacttccgcaggaaggaaaaggaacctgtattcttgtaggtggtcatgaaatcacttctggattagaagtaatttcttccctaagagcaattcatgggttgcaagtagaagtttgtcctcttaatggctgtgattacatcgtgagtaatcgcatggtggtggaaaggaggtctcaatctgagatgttaaatagtgtcaataagaacaagttcattgagcagatccagcacctgcagagtatgtttgaaagaatatgtgtgattgtggaaaaggacagagaaaaaacaggagacacatcaaggatgtttaggagaacaaagagctatgacagcctgctgactaccttaattggcgctggaatccgaattcttttcagttcctgccaagaagaaaccgcagatttgctaaaggaactgtctttagtggaacaaagaaagaatgttggtattcatgttccaacagtggtgaatagtaataaaagtgaggcactccagttttatttaagtattcccaatataagttatataactgcattaaatatgtgtcaccagttttcatctgtgaaaaggatggctaacagctcacttcaagaaatctccatgtatgcacaagtaactcatcagaaggctgaggagatctatagatatattcactatgtatttgacatacaaatgttaccaaatgatcttaaccaagatagactgaaatctgatatataatcaagctgctcaagatggggttttcaaagacctctcacaatattaaatgcacttcaataatcattgctgttttatgtttatttgtaaataagagaatattttatttaaatattttatattgtatacatttttatttatagattatagaaattattaaaaaagaaaaatctgatgttcagtgatcattttgactagattataaaactaatttttcttattaaataaaacaaggtttattaaaagtgttactaaggatagtttaagaaagtaaaagctaagctagagatatactttggaatgtttcccaaaattaaagttgtactgttgtgataaatagtaaagttgacatgtctatgactacagccaacttgtcgattttccctatgtgtagatagtatacttttaagtgtactgattctaaatacatgtacttggtaaggtgtgggtgatgggtgggttgtgagataaatgacccagtaactaggaaagtagaaaacttaactgaatgtttatctgaccaaaggtgtgtcccagttaagtactgtcaaatctattaatatgaactctgatatggtttggctgtgtccccaaccaaaatctcatcttgacttgtaatctgaattataatcccaatatattggggagggacctcctggaacgtgattagctcatgggggcggttcccccatgctgttctagtgatagttctcagaggatctgatggttttataagcttttcctctgttcactctgcagttctcttgcctactgccatgtggaaaaggaaacgtttgcttcccctccaccatgattgtaagttcccgaggcctccccagccatgcaggactgtgagtcaattaaacatcttttccttataaattaaaaaaaaaaaaaaaaa

SEQ ID NO: 9 - protein, 669 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGK

SEQ ID NO: 10 - DNA, 2139 bp, Homo sapiens
tgtgcgaaggaaaccgatggggatcggaaccgtagcggttgagctgctgctgctacggatatctgacagaagccttcggtggttgtcggcctaatgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggttgtaagagaactagtcaaatatacaaatcactttagaatcttggctctaagtgccacaccaggtagtgatataaaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggtaaataaattttgcatttgacacatgcaaaaaaaaaaaaaaaa

SEQ ID NO: 11 - protein, 2022 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 12 - DNA, 7053 bp, Homo sapiens
ccagagttttgtgcgaaggaaaccgatggggatcggaaccgtagcggttgagctgctgctgctacggatatctgacagaagccttcggtggttgtcggcctaatgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagctcaagaatcaaccactggaattcatcaactctctctctctgaatggagactgtggcaagatcatcctttgcctacacatcaagttgatcactcagatcgatgccgccattttataggccttatgcaaatgatagagggaatgagacacgaagagggagaatgcagctatgaattggaagttgaatcttatttacaaatggaagatgttacctcaacatttattgctcccaggaatgaatctaataatcttgccagtgacacctttatcactcacaagaaatcgtcatttataaagaacataaatcaaggcagttcatcctcagtgatagaatctgatgaagaatgtgctgaaattgttaaacaaactcatatcaaacctactaaaattgtttctttaaagaaaaaagtgtctaaagaaataaaaaaagatcagcttaaaaaagaaaataatcacggtattatagattctgtagataatgacagaaattccactgttgaaaatatttttcaagaagacctaccaaatgataaaaggacatcagatacagatgaaattgctgccacatgtactattaatgaaaatgttattaaagaaccgtgtgtgttattaacagagtgtcagtttacaaataaatccactagttcacttgctggaaatgttttagattctggttataacagtttcaatgatgaaaaatctgtttcatctaacttatttcttccattcgaagaagagctttatattgttagaacagatgaccaattttataattgtcactcattgacaaaagaggtactagctaatgtagagagatttttatcttattctcctccgcctctcagtggactctcagacttggaatatgaaattgctaagggtactgcacttgagaatttgcttttcttaccctgtgcagagcatttacgaagtgataaatgcacctgtttgctgtcacattcagctgtgaattctcaacagaatttagaattgaattcacttaaatgtataaattatccatctgaaaaaagttgcctttatgatatacctaatgataatatttctgatgagccaagtctctgtgactgtgatgtacataaacataatcaaaatgaaaatttagtacctaacaatcgtgttcaaatacacagaagccctgcacagaatttagttggagagaacaatcatgatgttgataacagtgacctcccagtattgtccactgatcaagatgaaagtttgctgttatttgaagatgttaatacagagttcgacgatgtgagtctttcacccttgaacagtaaaagcgaatctttacctgtgtcagacaaaactgctattagtgaaacgcctctggtctctcagttcttaatttctgatgaacttttgttggacaataattctgaactccaagatcaaatcacccgtgatgctaatagttttaaatctcgtgatcagagaggtgtacaggaagaaaaagtgaagaatcatgaggatatttttgattgctctagggatttattttctgttacctttgatttaggattctgtagtccagattctgatgatgaaatattggaacatacatcagatagcaatagacctctagatgatctatatggaaggtatttggaaattaaggagataagtgatgcaaattatgtttcgaatcaagcactaataccaagagatcatagtaaaaattttactagtggaactgttattatcccatcaaatgaagatatgcagaatccaaattatgtacatttgccactgagtgcagcaaaaaatgaagaattgttatctcctggttattctcagttttctttaccagtgcaaaaaaaagttatgagtacaccactctctaaatcaaacacattgaactcattttctaagataagaaaggaaatacttaagacaccagattctagtaaggaaaaagtaaacctacaaagattcaaagaagcattgaattcaacttttgattattcagaattttctctagaaaagtctaaaagcagtggtccaatgtatctgcataaatcctgtcattctgttgaagatggacaattattaacaagtaacgaaagtgaagatgacgagattttccgaagaaaagttaaaagagcaaaaggaaatgttttaaactctcctgaggatcagaaaaatagtgaagttgattctccacttcatgctgtcaaaaagcgcagatttcctataaacagatcagaattatcatctagtgatgagagtgagaattttcccaaaccatgttcacaattagaagacttcaaggtttgtaacgggaatgccagaagaggcatcaaagtcccaaagagacagagtcacttaaagcatgtagctaggaagtttttagatgatgaagcagaactttctgaagaagatgcagaatatgtttcatcagatgaaaatgatgagtcagaaaatgaacaagattcctcattacttgactttttaaatgatgaaactcaactttcacaggctataaatgattctgaaatgagagctatttacatgaaatctttgcgtagtccaatgatgaacaataagtacaaaatgattcataagacacataaaaacataaacattttctcgcagattcctgaacaagatgaaacctatttagaggatagtttttgtgttgatgaagaggagtcttgcaaaggccaatcaagtgaagaagaagtttgtgttgattttaacttaataactgatgattgctttgcaaatagtaaaaagtataaaactcgacgtgcagtaatgctaaaagaaatgatggaacaaaattgtgcacattcaaaaaagaaattatccagaattattttaccagatgattcaagtgaggaggagaacaatgtaaatgataaaagagaatctaatattgcggttaacccaagcactgttaagaagaacaaacaacaggaccattgtttaaattcagtgccttctggatcttctgcgcagtccaaggtgcgttctactccaagagttaatccattagcaaagcagagcaaacagacatcgctgaatttaaaggatacaatttccgaagtctcagacttcaaacctcagaatcataatgaagtccagtctaccacaccacccttcactactgttgattcacagaaagactgtagaaaatttccagttccacagaaggatggtagtgctttggaggattctagcacttcaggggcatcctgttccaagtcaagaccacatttagctgggacacatacttctcttagacttccgcaggaaggaaaaggaacctgtattcttgtaggtggtcatgaaatcacttctggattagaagtaatttcttccctaagagcaattcatgggttgcaagtagaagtttgtcctcttaatggctgtgattacatcgtgagtaatcgcatggtggtggaaaggaggtctcaatctgagatgttaaatagtgtcaataagaacaagttcattgagcagatccagcacctgcagagtatgtttgaaagaatatgtgtgattgtggaaaaggacagagaaaaaacaggagacacatcaaggatgtttaggagaacaaagagctatgacagcctgctgactaccttaattggcgctggaatccgaattcttttcagttcctgccaagaagaaaccgcagatttgctaaaggaactgtctttagtggaacaaagaaagaatgttggtattcatgttccaacagtggtgaatagtaataaaagtgaggcactccagttttatttaagtattcccaatataagttatataactgcattaaatatgtgtcaccagttttcatctgtgaaaaggatggctaacagctcacttcaagaaatctccatgtatgcacaagtaactcatcagaaggctgaggagatctatagatatattcactatgtatttgacatacaaatgttaccaaatgatcttaaccaagatagactgaaatctgatatataatcaagctgctcaagatggggttttcaaagacctctcacaatattaaatgcacttcaataatcattgctgttttatgtttatttgtaaataagagaatattttatttaaatattttatattgtatacatttttatttatagattatagaaattattaaaaaagaaaaatctgatgttcagtgatcattttgactagattataaaactaatttttcttattaaataaaacaaggtttattaaaagtgttactaaggatagtttaagaaagtaaaagctaagctagagatatactttggaatgtttcccaaaattaaagttgtactgttgtgataaatagtaaagttgacatgtctatgactacagccaacttgtcgattttccctatgtgtagatagtatacttttaagtgtactgattctaaatacatgtacttggtaaggtgtgggtgatgggtgggttgtgagataaatgacccagtaactaggaaagtagaaaacttaactgaatgtttatctgaccaaaggtgtgtcccagttaagtactgtcaaatctattaatatgaactctgatatggtttggctgtgtccccaaccaaaatctcatcttgacttgtaatctgaattataatcccaatatattggggagggacctcctggaacgtgattagctcatgggggcggttcccccatgctgttctagtgatagttctcagaggatctgatggttttataagcttttcctctgttcactctgcagttctcttgcctactgccatgtggaaaaggaaacgtttgcttcccctccaccatgattgtaagttcccgaggcctccccagccatgcaggactgtgagtcaattaaacatcttttccttataaatta

SEQ ID NO: 13 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 14 - protein, 63 aa, Homo sapiens
MDLSALRVEEVQNVINAMQKILECPICLELIKEPVSTKCDHIFCKFCMLKLLNQKKGPSQCPL

SEQ ID NO: 15 - protein, 759 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 16 - protein, 1846 aa, Homo sapiens
MQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 17 - protein, 721 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 18 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 19 - protein, 1884 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKDSHIHGQRNNSMFSKRPREHISVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 20 - protein, 1816 aa, Homo sapiens
MLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 21 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVANQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETSYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 22 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 23 - protein, 242 aa, Homo sapiens
MAMQMQLEANADTSVEEESFGPQPISRLEQCGINANDVKKLEEAGFHTVEAVAYAPKKELINIKGISEAKADKILAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 24 - protein, 280 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEIVSEERKRGNQNLQNLRLSLSS

SEQ ID NO: 25 - protein, 340 aa, Homo sapiens
MAMQMQLEANADTSVEEESFGPQPISRLEQCGINANDVKKLEEAGFHTVEAVAYAPKKELINIKGISEAKADKILTESRSVARLECNSVILVYCTLRLSGSSDSPASASRVVGTTGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 26 - protein, 376 aa, Homo sapiens
MRGKTFRFEMQRDLVSFPLSPAVRVKLVSAGFQTAEELLEVKPSELSKEVGISKAEALETLQIIRRECLTNKPRYAGTSESHKKCTALELLEQEHTQGFIITFCSALDDILGGGVPLMKTTEICGAPGVGKTQLCMQLAVDVQIPECFGGVAGEAVFIDTEGSFMVDRVVDLATACIQHLQLIAEKHKGEEHRKALEDFTLDNILSHIYYFRCRDYTELLAQVYLLPDFLSEHSKVRLVIVDGIAFPFRHDLDDLSLRTRLLNGLAQQMISLANNHRLAVILTNQMTTKIDRNQALLVPALGESWGHAATIRLIFHWDRKQRLATLYKSPSQKECTVLFQIKPQGFRDTVVTSACSLQTEGSLSTRKRSRDPEEEL

SEQ ID NO: 27 - protein, 135 aa, Homo sapiens
MRGKTFRFEMQRDLVSFPLSPAVRVKLVSAGFQTAEELLEVKPSELSKEVGISKAEALETLQIIRRECLTNKPRYAGTSESHKKCTALELLEQEHTQGFIITFCSALDDILGGGVPLMKTTEICGAPGVGKTQLW

SEQ ID NO: 28 - protein, 328 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGSLDKLLDAGLYTGEVTEIVGGPGSGKTQVCLCMAANVAHGLQQNVLYVDSNGGLTASRLLQLLQAKTQDEEEQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 29 - protein, 49 aa, Homo sapiens
MGVLRVGLCPGLTEEMIQLLRSHRIKTVVDLVSADLEEVAQKCGLSYKS

SEQ ID NO: 30 - protein, 216 aa, Homo sapiens
MGVLRVGLCPGLTEEMIQLLRSHRIKTVVDLVSADLEEVAQKCGLSYKAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 31 - protein, 283 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGSLDKLLDAGLYTGEVTEIVGGPGSGKTQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 32 - protein, 328 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGRQKLSGGSRWCMHLVTEIVGGPGSGKTQVCLCMAANVAHGLQQNVLYVDSNGGLTASRLLQLLQAKTQDEEEQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 33 - protein, 118 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGRHGGRTQVGTWEDCSCLRSPQGDRGVGSGML

SEQ ID NO: 34 - protein, 212 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGSLDKLLDAGLYTGEVTEIVGGPGSGKTQVCLCMAANVAHGLQQNVLYVDSNGGLTASRLLQLLQAKTQDEEEQAEALRRIQVVHAFDIFQMLDVLQELRGTVAQQDGIPEHLNHIPHCLHVHLPC

SEQ ID NO: 35 - protein, 348 aa, Homo sapiens
MGVLRVGLCPGLTEEMIQLLRSHRIKTVVDLVSADLEEVAQKCGLSYKTWRAHSSGNLGGLQLPQVPAGRSWSGVRNALKKAGLGHGGTDGLSLNAFDERGTAVSTSRLDKLLDAGLYTGEVTEIVGGPGSGKTQVCLCMAANVAHGLQQNVLYVDSNGGLTASRLLQLLQAKTQDEEEQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 36 - protein, 280 aa, Homo sapiens
MCSAFHRAESGTELLARLEGRSSLKEIEPNLFADEDSPVHGDILEFHGPEGTGKTEMLYHLTARCILPKSEGGLEVEVLFIDTDYHFDMLRLVTILEHRLSQSSEEIIKYCLGRFFLVYCSSSTHLLLTLYSLESMFCSHPSLCLLILDSLSAFYWIDRVNGGESVNLQESTLRKCSQCLEKLVNDYRLVLFATTQTIMQKASSSSEEPSHASRRLCDVDIDYRPYLCKAWQQLVKHRMFFSKQDDSQSSNQFSLVSRCLKSNSLKKHFFIIGESGVEFC

SEQ ID NO: 37 - protein, 916 aa, Homo sapiens
MESGQPARRIAMAPLLEYERQLVLELLDTDGLVVCARGLGADRLLYHFLQLHCHPACLVLVLNTQPAEEEYFINQLKIEGVEHLPRRVTNEITSNSRYEVYTQGGVIFATSRILVVDFLTDRIPSDLITGILVYRAHRIIESCQEAFILRLFRQKNKRGFIKAFTDNAVAFDTGFCHVERVMRNLFVRKLYLWPRFHVAVNSFLEQHKPEVVEIHVSMTPTMLAIQTAILDILNACLKELKCHNPSLEVEDLSLENAIGKPFDKTIRHYLDPLWHQLGAKTKSLVQDLKILRTLLQYLSQYDCVTFLNLLESLRATEKAFGQNSGWLFLDSSTSMFINARARVYHLPDAKMSKKEKISEKMEIKEGEETKKELVLESNPKWEALTEVLKEIEAENKESEALGGPGQVLICASDDRTCSQLRDYITLGAEAFLLRLYRKTFEKDSKAEEVWMKFRKEDSSKRIRKSHKRPKDPQNKERASTKERTLKKKKRKLTLTQMVGKPEELEEEGDVEEGYRREISSSPESCPEEIKHEEFDVNLSSDAAFGILKEPLTIIHPLLGCSDPYALTRVLHEVEPRYVVLYDAELTFVRQLEIYRASRPGKPLRVYFLIYGGSTEEQRYLTALRKEKEAFEKLIREKASMVVPEEREGRDETNLDLVRGTASADVSTDTRKAGGQEQNGTQQSIVVDMREFRSELPSLIHRRGIDIEPVTLEVGDYILTPEMCVERKSISDLIGSLNNGRLYSQCISMSRYYKRPVLLIEFDPSKPFSLTSRGALFQEISSNDISSKLTLLTLHFPRLRILWCPSPHATAELFEELKQSKPQPDAATALAITADSETLPESEKYNPGPQDFLLKMPGVNAKNCRSLMHHVKNIAELAALSQDELTSILGNAANAKQLYDFIHTSFAEVVSKGKGKK

SEQ ID NO: 38 - protein, 372 aa, Homo sapiens
MESGQPARRIAMAPLLEYERQLVLELLDTDGLVVCARGLGADRLLYHFLQLHCHPACLVLVLNTQPAEEEYFINQLKIEGVEHLPRRVTNEITSNSRYEVYTQGGVIFATSRILVVDFLTDRIPSDLITGILVYRAHRIIESCQEAFILRLFRQKNKRGFIKAFTDNAVAFDTGFCHVERVMRNLFVRKLYLWPRFHVAVNSFLEQHKPEVVEIHVSMTPTMLAIQTAILDILNACLKELKCHNPSLEVEDLSLENAIGKPFDKTIRHYLDPLWHQLGAKTKSLVQDLKILRTLLQYLSQYDCVTFLNLLESLRATEKAFGQNSGWLFLDSSTSMFINARARVYHLPDAKMSKKEKISEKMEIKEGEGILWG

SEQ ID NO: 39 - protein, 708 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 40 - protein, 680 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 41 - protein, 711 aa, Homo sapiens
MNRNISHQKGDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 42 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 43 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRRVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 44 - protein, 758 aa, Homo sapiens
MPDNRQPRNRQPRIRSGNEPRSAPAMEPDGRGAWAHSRAALDRLEKLLRCSRCNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRRVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 45 - protein, 680 aa, Homo sapiens
MVAVPGPTVAPRSTAWRSCCAARVDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRRVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 46 - protein, 127 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSGRHTFC

SEQ ID NO: 47 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTSPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 48 - protein, 994 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTSPYLLEAASHLSPENFVEDEAKICVQELQCPKIITKNSPLPSSIISRKEKSMKSSSHLPLIEKSFIIFSEMIFIWV

SEQ ID NO: 49 - protein, 476 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKIWLPAT

SEQ ID NO: 50 - protein, 382 aa, Homo sapiens
MEKPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKIWLPAT

SEQ ID NO: 51 - protein, 442 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQGDGLEFKRHFLKIKGKLIDIVSSQKIWLPAT

SEQ ID NO: 52 - protein, 543 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 53 - protein, 234 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKEKILKIYSLSRFSKIRRGAVAHVFNPSTLGGRGWQIT

SEQ ID NO: 54 - protein, 162 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLDEDMKRKFQDLLSEENESTALPQVLAQPSTSRKRPREGEAEGAETTKRPAVCAAVL

SEQ ID NO: 55 - protein, 452 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 56 - protein, 203 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVVPVER

SEQ ID NO: 57 - protein, 165 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREENLSCPYRIWFNFCLF

SEQ ID NO: 58 - protein, 339 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQMKT

SEQ ID NO: 59 - protein, 289 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHDGRGRAV

SEQ ID NO: 60 - protein, 586 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLETESGHVTQSDLELLLSSDPPASASQSAGIRGVRHHPRPVCSLKCVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 61 - protein, 140 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLEFRSYSFYLP

SEQ ID NO: 62 - protein, 225 aa, Homo sapiens
MSRESDVEAQQSHGSSACSQPHGSVTQSQGSSSQSQGISSSSTSTMPNSSQSSHSSSGTLSSLETVSTQELYSIPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 63 - protein, 514 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 64 - protein, 322 aa, Homo sapiens
MSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 65 - protein, 754 aa, Homo sapiens
MWKLLPAAGPAGGEPYRLLTGVEYVVGRKNCAILIENDQSISRNHAVLTANFSVTNLSQTDEIPVLTLKDNSKYGTFVNEEKMQNGFSRTLKSGDGITFGVFGSKFRIEYEPLVACSSCLDVSGKTALNQAILQLGGFTVNNWTEECTHLVMVSVKVTIKTICALICGRPIVKPEYFTEFLKAVESKKQPPQIESFYPPLDEPSIGSKNVDLSGRQERKQIFKGKTFIFLNAKQHKKLSSAVVFGGGEARLITEENEEEHNFFLAPGTCVVDTGITNSQTLIPDCQKKWIQSIMDMLQRQGLRPIPEAEIGLAVIFMTTKNYCDPQGHPSTGLKTTTPGPSLSQGVSVDEKLMPSAPVNTTTYVADTESEQADTWDLSERPKEIKVSKMEQKFRMLSQDAPTVKESCKTSSNNNSMVSNTLAKMRIPNYQLSPTKLPSINKSKDRASQQQQTNSIRNYFQPSTKKRERDEENQEMSSCKSARIETSCSLLEQTQPATPSLWKNKEQHLSENEPVDTNSDNNLFTDTDLKSIVKNSASKSHAAEKLRSNKKREMDDVAIEDEVLEQLFKDTKPELEIDVKVQKQEEDVNVRKRPRMDIETNDTFSDEAVPESSKISQENEIGKKRELKEDSLWSAKEISNNDKLQDDSEMLPKKLLLTEFRSLVIKNSTSRNPSGINDDYGQLKNFKKFKKVTYPGAGKLPHIIGGSDLIAHHARKNTELEEWLRQEMEVQNQHAKEESLADDLFRYNPYLKRRR

SEQ ID NO: 66 - protein, 1186 aa, Homo sapiens [sequence omitted]

SEQ ID NO: 67 - protein, 1834 aa, Homo sapiens
MKLSVNEAQLGFYLGSLSHLSACPGIDPRSSEDQPESLKTGQMMDESDEDFKELCASFFQRVKKHGIKEVSGERKTQKAASNGTQIRSKLKRTKQTATKTKTLQGPAEKKPPSGSQAPRTKKQRVTKWQASEPAHSVNGEGGVLASAPDPPVLRETAQNTQTGNQQEPSPNLSREKTRENVPNSDSQPPPSCLTTAVPSPSKPRTAQLVLQRMQQFKRADPERLRHASEECSLEAAREENVPKDPQEEMMAGNVYGLGPPAPESDAAVALTLQQEFARVGASAHDDSLEEKGLFFCQICQKNLSAMNVTRREQHVNRCLDEAEKTLRPSVPQIPECPICGKPFLTLKSRTSHLKQCAVKMEVGPQLLLQAVRLQTAQPEGSSSPPMFSFSDHSRGLKRRGPTSKKEPRKRRKVDEAPSEDLLVAMALSRSEMEPGAAVPALRLESAFSERIRPEAENKSRKKKPPVSPPLLLVQDSETTGRQIEDRVALLLSEEVELSSTPPLPASRILKEGWERAGQCPPPPERKQSFLWEGSALTGAWAMEDFYTARLVPPLVPQRPAQGLMQEPVPPLVPPEHSELSERRSPALHGTPTAGCGSRGPSPSASQREHQALQDLVDLAREGLSASPWPGSGGLAGSEGTAGLDVVPGGLPLTGFVVPSQDKHPDRGGRTLLSLGLLVADFGAMVNNPHLSDVQFQTDSGEVLYAHKFVLYARCPLLIQYVNNEGFSAVEDGVLTQRVLLGDVSTEAARTFLHYLYTADTGLPPGLSSELSSLAHRFGVSELVHLCEQVPIATDSEGKPWEEKEAENCESRAENFQELLRSMWADEEEEAETLLKSKDHEEDQENVNEAEMEEIYEFAATQRKLLQEERAAGAGEDADWLEGGSPVSGQLLAGVQVQKQWDKVEEMEPLEPGRDEAATTWEKMGQCALPPPQGQHSGARGAEAPEQEAPEEALGHSSCSSPSRDCQAERKEGSLPHSDDAGDYEQLFSSTQGEISEPSQITSEPEEQSGAVRERGLEVSHRLAPWQASPPHPCRFLLGPPQGGSPRGSHHTSGSSLSTPRSRGGTSQVGSPTLLSPAVPSKQKRDRSILTLSKEPGHQKGKERRSVLECRNKGVLMFPEKSPSIDLTQSNPDHSSSRSQKSSSKLNEEDEVILLLDSDEELELEQTKMKSISSDPLEEKKALEISPRSCELFSIIDVDADQEPSQSPPRSEAVLQQEDEGALPENRGSLGRRGAPWLFCDRESSPSEASTTDTSWLVPATPLASRSRDCSSQTQISSLRSGLAVQAVTQHTPRASVGNREGNEVAQKFSVIRPQTPPPQTPSSCLTPVSPGTSDGRRQGHRSPSRPHPGGHPHSSPLAPHPISGDRAHFSRRFLKHSPPGPSFLNQTPAGEVVEVGDSDDEQEVASHQANRSPPLDSDPPIPIDDCCWHMEPLSPIPIDHWNLERTGPLSTSSPSRRMNEAADSRDCRSPGLLDTTPIRGSCTTQRKLQEKSSGAGSLGNSRPSFLNSALWDVWDGEEQRPPETPPPAQMPSAGGAQKPEGLETPKGANRKKNLPPKVPITPMPQYSIMETPVLKKELDRFGVRPLPKRQMVLKLKEIFQYTHQTLDSDSEDESQSSQPLLQAPHCQTLASQTYKPSRAGVHAQQEATTGPGAHRPKGPAKTKGPRHQRKHHESITPPSRSPTKEAPPGLNDDAQIPASQESVATSVDGSDSSLSSQSSSSCEFGAAFESAGEEEGEGEVSASQAAVQAADTDEALRCYIRSKPALYQKVLLYQPFELRELQAELRQNGLRVSSRRLLDFLDTHCITFTTAATRREKLQGRRRQPRGKKKVERN

SEQ ID NO: 68 - protein, 1522 aa, Homo sapiens
MFSFRCLDEAEKTLRPSVPQIPECPICGKPFLTLKSRTSHLKQCAVKMEVGPQLLLQAVRLQTAQPEGSSSPPMFSFSDHSRGLKRRGPTSKKEPRKRRKVDEAPSEDLLVAMALSRSEMEPGAAVPALRLESAFSERIRPEAENKSRKKKPPVSPPLLLVQDSETTGRQIEDRVALLLSEEVELSSTPPLPASRILKEGWERAGQCPPPPERKQSFLWEGSALTGAWAMEDFYTARLVPPLVPQRPAQGLMQEPVPPLVPPEHSELSERRSPALHGTPTAGCGSRGPSPSASQREHQALQDLVDLAREGLSASPWPGSGGLAGSEGTAGLDVVPGGLPLTGFVVPSQDKHPDRGGRTLLSLGLLVADFGAMVNNPHLSDVQFQTDSGEVLYAHKFVLYARCPLLIQYVNNEGFSAVEDGVLTQRVLLGDVSTEAARTFLHYLYTADTGLPPGLSSELSSLAHRFGVSELVHLCEQVPIATDSEGKPWEEKEAENCESRAENFQELLRSMWADEEEEAETLLKSKDHEEDQENVNEAEMEEIYEFAATQRKLLQEERAAGAGEDADWLEGGSPVSGQLLAGVQVQKQWDKVEEMEPLEPGRDEAATTWEKMGQCALPPPQGQHSGARGAEAPEQEAPEEALGHSSCSSPSRDCQAERKEGSLPHSDDAGDYEQLFSSTQGEISEPSQITSEPEEQSGAVRERGLEVSHRLAPWQASPPHPCRFLLGPPQGGSPRGSHHTSGSSLSTPRSRGGTSQVGSPTLLSPAVPSKQKRDRSILTLSKEPGHQKGKERRSVLECRNKGVLMFPEKSPSIDLTQSNPDHSSSRSQKSSSKLNEEDEVILLLDSDEELELEQTKMKSISSDPLEEKKALEISPRSCELFSIIDVDADQEPSQSPPRSEAVLQQEDEGALPENRGSLGRRGAPWLFCDRESSPSEASTTDTSWLVPATPLASRSRDCSSQTQISSLRSGLAVQAVTQHTPRASVGNREGNEVAQKFSVIRPQTPPPQTPSSCLTPVSPGTSDGRRQGHRSPSRPHPGGHPHSSPLAPHPISGDRAHFSRRFLKHSPPGPSFLNQTPAGEVVEVGDSDDEQEVASHQANRSPPLDSDPPIPIDDCCWHMEPLSPIPIDHWNLERTGPLSTSSPSRRMNEAADSRDCRSPGLLDTTPIRGSCTTQRKLQEKSSGAGSLGNSRPSFLNSALWDVWDGEEQRPPETPPPAQMPSAGGAQKPEGLETPKGANRKKNLPPKVPITPMPQYSIMETPVLKKELDRFGVRPLPKRQMVLKLKEIFQYTHQTLDSDSEDESQSSQPLLQAPHCQTLASQTYKPSRAGVHAQQEATTGPGAHRPKGPAKTKGPRHQRKHHESITPPSRSPTKEAPPGLNDDAQIPASQESVATSVDGSDSSLSSQSSSSCEFGAAFESAGEEEGEGEVSASQAAVQAADTDEALRCYIRSKPALYQKVLLYQPFELRELQAELRQNGLRVSSRRLLDFLDTHCITFTTAATRREKLQGRRRQPRGKKKVERN

SEQ ID NO: 69 - DNA, 20 bp, Artificial Sequence
  Synthesized single guide RNA sequence
catgaccacggcggcaataa

SEQ ID NO: 70 - DNA, 20 bp, Artificial Sequence
  Synthesized single guide RNA sequence
aaagacctttattgccgccg

SEQ ID NO: 71 - DNA, 20 bp, Artificial Sequence
  Synthesized single guide RNA sequence
cctttcctgaagggaaccag

SEQ ID NO: 72 - DNA, 20 bp, Artificial Sequence
  Synthesized single guide RNA sequence
attatgctgaggatttggaa

SEQ ID NO: 73 - DNA, 20 bp, Artificial Sequence
  Synthesized single guide RNA sequence
agtctcgaagatatgactcc

SEQ ID NO: 74 - DNA, 20 bp, Artificial Sequence
  Synthesized single guide RNA sequence
cgaagataacgcggatacct

SEQ ID NO: 75 - DNA, 20 bp, Artificial Sequence
  Synthesized single guide RNA sequence
gtacgtcggtataactcctc

SEQ ID NO: 76 - DNA, 21 bp, Artificial Sequence
  Synthesized short hairpin RNA
caacaagatgaagagcaccaa

SEQ ID NO: 77 - DNA, 21 bp, Artificial Sequence
  Synthesized short hairpin RNA
gacttcatgaaactctataat

SEQ ID NO: 78 - DNA, 21 bp, Artificial Sequence
  Synthesized short hairpin RNA
aggacgagaggaacgtattta